SEQUENCE LISTING (348 sequences)

SEQ ID NO: 1 - protein, 16 aa, Artificial Sequence
  Chemically Synthesized
NDECELCVNVACTGCL

SEQ ID NO: 2 - protein, 15 aa, Artificial Sequence
  Chemically Synthesized
DECELCVNVACTGCL

SEQ ID NO: 3 - protein, 14 aa, Artificial Sequence
  Chemically Synthesized
DECELCVNVACTGC

SEQ ID NO: 4 - protein, 14 aa, Artificial Sequence
  Chemcially Synthesized
ECELCVNVACTGCL

SEQ ID NO: 5 - protein, 13 aa, Artificial Sequence
  Chemically Synthesized
ECELCVNVACTGC

SEQ ID NO: 6 - protein, 13 aa, Artificial Sequence
  Chemically Synthesized
CELCVNVACTGCL

SEQ ID NO: 7 - protein, 12 aa, Artificial Sequence
  Chemically Synthesized
CELCVNVACTGC

SEQ ID NO: 8 - protein, 16 aa, Artificial Sequence
  Chemically Synthesized
  MISC_FEATURE (16)..(16): wherein LEU is a D amino acid
NDECELCVNVACTGCL

SEQ ID NO: 9 - protein, 16 aa, Artificial Sequence
  Chemically Synthesized
  MISC_FEATURE (1)..(1): wherein ASN is a D amino acid
  MISC_FEATURE (16)..(16): wherein LEU is a D amino acid
NDECELCVNVACTGCL

SEQ ID NO: 10 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (16)..(16): wherein LEU is a D amino acid
NDECELCVNVACTGCL

SEQ ID NO: 11 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (16)..(16): wherein LEU is a D amino acid
NDECELCVNVACTGCL

SEQ ID NO: 12 - protein, 16 aa, Artificial Sequence
  Chemically Synthesized
  MISC_FEATURE (1)..(1): wherein ASN is a D amino acid
NDECELCVNVACTGCL

SEQ ID NO: 13 - protein, 16 aa, Artificial Sequence
  Chemically Synthesized
  MISC_FEATURE (1)..(1): wherein ASN is a D amino acid
  MISC_FEATURE (6)..(6): wherein LEU is a D amino acid
  MISC_FEATURE (16)..(16): wherein LEU is a D amino acid
NDECELCVNVACTGCL

SEQ ID NO: 14 - protein, 15 aa, Artificial Sequence
  Chemically Synthesized
NDECELCVNVACTGC

SEQ ID NO: 15 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein LEU is a D amino acid
  MISC_FEATURE (16)..(16): wherein LEU at position 16 is attached to polyethylene glycol
NDECELCVNVACTGCL

SEQ ID NO: 16 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
  MISC_FEATURE (16)..(16): wherein LEU at position 16 is attached to polyethylene glycol
NDECELCVNVACTGCL

SEQ ID NO: 17 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (2)..(2): wherein ASP is a D amino acid
  MISC_FEATURE (16)..(16): wherein LEU is a D amino acid
  MISC_FEATURE (16)..(16): wherein LEU at position 16 is attached to polyethylene glycol
NDECELCVNVACTGCL

SEQ ID NO: 18 - protein, 16 aa, Artificial Sequence
  Chemically Synthesized
  MISC_FEATURE (1)..(1): wherein ASN is a D amino acid
  MISC_FEATURE (16)..(16): wherein LEU at position 16 is attached to polyethylene glycol
  MISC_FEATURE (16)..(16): wherein LEU is a D amino acid
NDECELCVNVACTGCL

SEQ ID NO: 19 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein LEU is a D amino acid
NDECELCVNVACTGCL

SEQ ID NO: 20 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
NDECELCVNVACTGCL

SEQ ID NO: 21 - protein, 16 aa, Artificial Sequence
  Chemically Synthesized
  MISC_FEATURE (16)..(16): wherein LEU at position 16 is attached to polyethylene glycol
  MISC_FEATURE (16)..(16): wherein LEU is a D amino acid
NDECELCVNVACTGCL

SEQ ID NO: 22 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (16)..(16): wherein LEU is a D amino acid
  MISC_FEATURE (16)..(16): wherein LEU at position 16 is attached to polyethylene glycol
NDECELCVNVACTGCL

SEQ ID NO: 23 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (16)..(16): wherein LEU is a D amino acid
NDECELCVNVACTGCL

SEQ ID NO: 24 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (16)..(16): wherein LEU at position 16 is attached to polyethylene glycol
  MISC_FEATURE (16)..(16): wherein LEU is a D amino acid
NDECELCVNVACTGCL

SEQ ID NO: 25 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (16)..(16): wherein LEU at position 16 is attached to polyethylene glycol
  MISC_FEATURE (16)..(16): wherein LEU is a D amino acid
NDECELCVNVACTGCL

SEQ ID NO: 26 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (2)..(2): wherein ASP is a D amino acid
  MISC_FEATURE (16)..(16): wherein LEU is a D amino acid
NDECELCVNVACTGCL

SEQ ID NO: 27 - protein, 16 aa, Artificial Sequence
  Chemically Synthesized
  MISC_FEATURE (1)..(1): wherein ASN is a D amino acid
  MISC_FEATURE (16)..(16): wherein x is 3-(2-naphthyl)alanine
  MISC_FEATURE (16)..(16): wherein x is a D amino acid
NDECELCVNVACTGCX

SEQ ID NO: 28 - protein, 16 aa, Artificial Sequence
  Chemically Synthesized
  MISC_FEATURE (1)..(1): wherein ASN is a D amino acid
  MISC_FEATURE (8)..(8): wherein x at position 8 is alpha-amino isobutyric acid
  MISC_FEATURE (10)..(10): wherein x at position 10 is alpha-amino isobutyric acid
  MISC_FEATURE (16)..(16): wherein LEU is a D amino acid
NDECELCXNXACTGCL

SEQ ID NO: 29 - protein, 16 aa, Artificial Sequence
  Chemically Synthesized
  MISC_FEATURE (1)..(1): wherein ASN is a D amino acid
  MISC_FEATURE (7)..(7): wherein ASP at position 7 is attached to a Lactam bridge
  MISC_FEATURE (15)..(15): wherein x at position 15 is ornithine
  MISC_FEATURE (16)..(16): wherein LEU is a D amino acid
NDECELDVNVACTGXL

SEQ ID NO: 30 - protein, 16 aa, Artificial Sequence
  Chemically Synthesized
  MISC_FEATURE (1)..(1): wherein ASN is a D amino acid
  MISC_FEATURE (16)..(16): wherein LEU is a D amino acid
NDECEYCVNVACTGCL

SEQ ID NO: 31 - protein, 16 aa, Artificial Sequence
  Chemically Synthesized
  MISC_FEATURE (1)..(1): wherein ASN is a D amino acid
  MISC_FEATURE (16)..(16): wherein LEU is a D amino acid
NDECESCVNVACTGCL

SEQ ID NO: 32 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
  MISC_FEATURE (16)..(16): wherein LEU at position 16 is attached to polyethylene glycol
NDECEYCVNVACTGCL

SEQ ID NO: 33 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
NDECEYCVNVACTGCL

SEQ ID NO: 34 - protein, 16 aa, Artificial Sequence
  Chemically Synthesized
  MISC_FEATURE (1)..(1): wherein ASN is a D amino acid
  MISC_FEATURE (16)..(16): wherein LEU at position 16 is attached to polyethylene glycol
  MISC_FEATURE (16)..(16): wherein LEU is a D amino acid
NDECEYCVNVACTGCL

SEQ ID NO: 35 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
  MISC_FEATURE (16)..(16): wherein LEU at position 16 is attached to polyethylene glycol
NDECESCVNVACTGCL

SEQ ID NO: 36 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
NDECESCVNVACTGCL

SEQ ID NO: 37 - protein, 16 aa, Artificial Sequence
  Chemically Synthesized
  MISC_FEATURE (1)..(1): wherein ASN is a D amino acid
  MISC_FEATURE (16)..(16): wherein LEU at position 16 is attached to polyethylene glycol
  MISC_FEATURE (16)..(16): wherein LEU is a D amino acid
NDECESCVNVACTGCL

SEQ ID NO: 38 - protein, 16 aa, Artificial Sequence
  Chemically Synthesized
NDECELCVNVACTGCS

SEQ ID NO: 39 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein SER at position 16 is attached to polyethylene glycol
NDECELCVNVACTGCS

SEQ ID NO: 40 - protein, 16 aa, Artificial Sequence
  Chemically Synthesized
  MISC_FEATURE (1)..(1): wherein ASN at position 1 is attached to polyethylene glycol
NDECELCVNVACTGCS

SEQ ID NO: 41 - protein, 16 aa, Artificial Sequence
  Chemically Synthesized
  MISC_FEATURE (16)..(16): wherein SER at position 16 is attached to polyethylene glycol
NDECELCVNVACTGCS

SEQ ID NO: 42 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein SER is a D amino acid
  MISC_FEATURE (16)..(16): wherein SER at position 16 is attached to polyethylene glycol
NDECELCVNVACTGCS

SEQ ID NO: 43 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein SER is a D amino acid
NDECELCVNVACTGCS

SEQ ID NO: 44 - protein, 16 aa, Artificial Sequence
  Chemically Synthesized
  MISC_FEATURE (16)..(16): wherein SER at position 16 is attached to polyethylene glycol
  MISC_FEATURE (16)..(16): wherein SER is a D amino acid
NDECELCVNVACTGCS

SEQ ID NO: 45 - protein, 16 aa, Artificial Sequence
  Chemically Synthesized
  MISC_FEATURE (5)..(6): x is natural, unnatural, an analogue, L, D, or methylated amino acid or any combination
  MISC_FEATURE (8)..(11): x is natural, unnatural, an analogue, L, D, or methylated amino acid or any combination
  MISC_FEATURE (13)..(14): x is natural, unnatural, an analogue, L, D, or methylated amino acid or any combination
  MISC_FEATURE (16)..(16): x is natural, unnatural, an analogue, L, D, or methylated amino acid or any combination
NDECXXCXXXXCXXCX

SEQ ID NO: 46 - protein, 16 aa, Artificial Sequence
  Chemically Synthesized
  MISC_FEATURE (1)..(1): x is none or a natural, unnatural or an analogue amino acid, may be L, D, or a methylated amino acid, or any combination.
  MISC_FEATURE (2)..(2): x is none or a natural, unnatural or an analogue amino acid, may be L, D, or a methylated amino acid, or any combination.
  MISC_FEATURE (3)..(3): x is any natural, unnatural or an analogue amino acid, may be L, D, or a methylated amino acid, or any combination.
  MISC_FEATURE (5)..(6): x is natural, unnatural, analogue, L, D, or methylated amino acid or any combination
  MISC_FEATURE (8)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is none or one natural, unnatural or an analogue amino acid, may be L, D, or a methylated amino acid, or any combination
XXXCXXCXXXXCXXCX

SEQ ID NO: 47 - protein, 16 aa, Artificial Sequence
  Chemically Synthesized
  MISC_FEATURE (1)..(1): x is none or a natural, unnatural or an analogue amino acid,  may be L, D, or a methylated amino acid or any combination thereof
  MISC_FEATURE (2)..(2): x is none or a natural, unnatural or an analogue amino acid,  may be L, D, or a methylated amino acid or any combination thereof
  MISC_FEATURE (3)..(3): x is any natural, unnatural or an analogue amino acid,  may be L, D, or a methylated amino acid or any combination thereof
  MISC_FEATURE (4)..(4): wherein x is a cysteine, penicillamine homocysteine, or 3-mercaptoproline
  MISC_FEATURE (6)..(6): x is natural, unnatural, analogue, L, D, or methylated amino acid or any combination
  MISC_FEATURE (7)..(7): wherein x is a cysteine, penicillamine homocysteine, or 3-mercaptoproline
  MISC_FEATURE (12)..(12): wherein x is a cysteine, penicillamine homocysteine, or 3-mercaptoproline
  MISC_FEATURE (15)..(15): wherein x is a cysteine, penicillamine homocysteine, or 3-mercaptoproline
  MISC_FEATURE (16)..(16): x is natural, unnatural or an analogue, zero or one residue in length, may be L, D, or a methylated amino acid or any combination thereof
XXXXEXXVNVAXTGXX

SEQ ID NO: 48 - protein, 16 aa, Artificial Sequence
  Chemically Synthesized
  MISC_FEATURE (1)..(1): x is none or a natural, unnatural or an analogue amino acid, may be L, D, or a methylated amino acid or any combination thereof
  MISC_FEATURE (2)..(2): x is none or a natural, unnatural or an analogue amino acid, may be L, D, or a methylated amino acid or any combination thereof
  MISC_FEATURE (3)..(3): x is any natural, unnatural or an analogue amino acid, may be L, D, or a methylated amino acid or any combination thereof
  MISC_FEATURE (4)..(4): wherein x is a cysteine, penicillamine homocysteine, or 3-mercaptoproline
  MISC_FEATURE (5)..(6): x is any natural, unnatural or an analogue amino acid, may be L, D, or a methylated amino acid or any combination thereof
  MISC_FEATURE (7)..(7): wherein x is a cysteine, penicillamine homocysteine, or 3-mercaptoproline
  MISC_FEATURE (8)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): wherein x is a cysteine, penicillamine homocysteine, or 3-mercaptoproline
  MISC_FEATURE (16)..(16): x is natural, unnatural or an analogue, zero or one residue in length, may be L, D, or a methylated amino acid or any combination thereof
XXXXXXXXXXXXXXXX

SEQ ID NO: 49 - protein, 16 aa, Artificial Sequence
  Chemically Synthesized
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is natural, unnatural, analogue, L, D, or methylated amino acid or any combination
NDDCXXCXNXXCXXCX

SEQ ID NO: 50 - protein, 16 aa, Artificial Sequence
  Chemically Synthesized
  MISC_FEATURE (1)..(1): wherein ASN is a D amino acid
  MISC_FEATURE (5)..(6): x is natural, unnatural, analogue, L, D, or methylated amino acid or any combination
  MISC_FEATURE (8)..(8): x is any natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is natural, unnatural, analogue, L, D, or methylated amino acid or any combination
  MISC_FEATURE (16)..(16): wherein x is a D amino acid
NEECXXCXNXXCXXCX

SEQ ID NO: 51 - protein, 16 aa, Artificial Sequence
  Chemically Synthesized
  MISC_FEATURE (1)..(1): wherein ASN is a D amino acid
  MISC_FEATURE (2)..(2): wherein GLU is a D amino acid
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): wherein x is a D amino acid
  MISC_FEATURE (16)..(16): x is natural, unnatural, analogue, L, D, or methylated amino acid or any combination
NEDCXXCXNXXCXXCX

SEQ ID NO: 52 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): wherein x is a D amino acid
  MISC_FEATURE (16)..(16): x is natural, unnatural, analogue, L, D, or methylated amino acid or any combination
NDECXXCXNXXCXXCX

SEQ ID NO: 53 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): wherein x is a D amino acid
  MISC_FEATURE (16)..(16): x is natural, unnatural, analogue, L, D, or methylated amino acid or any combination
NDECXXCXYXXCXXCX

SEQ ID NO: 54 - protein, 16 aa, Artificial Sequence
  Chemically Synthesized
  MISC_FEATURE (1)..(1): wherein ASN is a D amino acid
  MISC_FEATURE (2)..(2): wherein GLU is a D amino acid
  MISC_FEATURE (3)..(3): wherein GLU is a D amino acid
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): wherein x is a D amino acid
  MISC_FEATURE (16)..(16): x is natural, unnatural, analogue, L, D, or methylated amino acid or any combination
NEECXXCXYXXCXXCX

SEQ ID NO: 55 - protein, 14 aa, Artificial Sequence
  Chemically Synthesized
CCEYCCNPACTGCY

SEQ ID NO: 56 - protein, 13 aa, Artificial Sequence
  Chemically Synthesized
CCEYCCNPACTGC

SEQ ID NO: 57 - protein, 14 aa, Artificial Sequence
  Chemically Synthesized
  MISC_FEATURE (1)..(1): wherein CYS at position 1 is attached to polyethylene glycol
  MISC_FEATURE (14)..(14): wherein TYR at position 14 is attached to polyethylene glycol
CCEYCCNPACTGCY

SEQ ID NO: 58 - protein, 16 aa, Artificial Sequence
  Chemically Synthesized
NFCCESCCNPACTGCY

SEQ ID NO: 59 - protein, 16 aa, Artificial Sequence
  Chemically Synthesized
NFCCEFCCNPACTGCY

SEQ ID NO: 60 - protein, 14 aa, Artificial Sequence
  Chemically Synthesized
  MISC_FEATURE (14)..(14): wherein TYR is a D amino acid
CCEYCCNPACTGCY

SEQ ID NO: 61 - protein, 14 aa, Artificial Sequence
  Chemically Synthesized
  MISC_FEATURE (1)..(1): wherein CYS at position 1 is attached to polyethylene glycol
CCEYCCNPACTGCY

SEQ ID NO: 62 - protein, 16 aa, Artificial Sequence
  Chemically Synthesized
NFCCETCCNPACTGCY

SEQ ID NO: 63 - protein, 16 aa, Artificial Sequence
  Chemically Synthesized
  MISC_FEATURE (16)..(16): wherein TYR is a D amino acid
NFCCESCCNPACTGCY

SEQ ID NO: 64 - protein, 16 aa, Artificial Sequence
  Chemically Synthesized
  MISC_FEATURE (1)..(1): wherein ASN is a D amino acid
NFCCESCCNPACTGCY

SEQ ID NO: 65 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NFCCESCCNPACTGCY

SEQ ID NO: 66 - protein, 16 aa, Artificial Sequence
  Chemically Synthesized
  MISC_FEATURE (16)..(16): wherein TYR is a D amino acid
NFCCETCCNPACTGCY

SEQ ID NO: 67 - protein, 16 aa, Artificial Sequence
  Chemically Synthesized
  MISC_FEATURE (1)..(1): wherein ASN is a D amino acid
NFCCETCCNPACTGCY

SEQ ID NO: 68 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NFCCETCCNPACTGCY

SEQ ID NO: 69 - protein, 16 aa, Artificial Sequence
  Chemically Synthesized
  MISC_FEATURE (16)..(16): wherein TYR is a D amino acid
NFCCEFCCNPACTGCY

SEQ ID NO: 70 - protein, 16 aa, Artificial Sequence
  Chemically Synthesized
  MISC_FEATURE (1)..(1): wherein ASN is a D amino acid
NFCCEFCCNPACTGCY

SEQ ID NO: 71 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NFCCEFCCNPACTGCY

SEQ ID NO: 72 - protein, 14 aa, Artificial Sequence
  Chemically Synthesized
  MISC_FEATURE (14)..(14): wherein TYR at position 14 is attached to polyethylene glycol
CCEYCCNPACTGCY

SEQ ID NO: 73 - protein, 13 aa, Artificial Sequence
  Chemically Synthesized
  MISC_FEATURE (1)..(1): wherein CYS at position 1 is attached to polyethylene glycol
  MISC_FEATURE (13)..(13): wherein CYS at position 13 is attached to polyethylene glycol
CCEYCCNPACTGC

SEQ ID NO: 74 - protein, 13 aa, Artificial Sequence
  Chemically Synthesized
  MISC_FEATURE (1)..(1): wherein CYS at position 1 is attached to polyethylene glycol
CCEYCCNPACTGC

SEQ ID NO: 75 - protein, 13 aa, Artificial Sequence
  Chemically Synthesized
  MISC_FEATURE (13)..(13): wherein CYS at position 13 is attached to polyethylene glycol
CCEYCCNPACTGC

SEQ ID NO: 76 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein TYR at position 16 is attached to polyethylene glycol
NFCCESCCNPACTGCY

SEQ ID NO: 77 - protein, 16 aa, Artificial Sequence
  Chemically Synthesized
  MISC_FEATURE (1)..(1): wherein ASN at position 1 is attached to polyethylene glycol
NFCCESCCNPACTGCY

SEQ ID NO: 78 - protein, 16 aa, Artificial Sequence
  Chemically Synthesized
  MISC_FEATURE (16)..(16): wherein TYR at position 16 is attached to polyethylene glycol
NFCCESCCNPACTGCY

SEQ ID NO: 79 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein TYR at position 16 is attached to polyethylene glycol
NFCCEFCCNPACTGCY

SEQ ID NO: 80 - protein, 16 aa, Artificial Sequence
  Chemically Synthesized
  MISC_FEATURE (1)..(1): wherein ASN at position 1 is attached to polyethylene glycol
NFCCEFCCNPACTGCY

SEQ ID NO: 81 - protein, 16 aa, Artificial Sequence
  Chemically Synthesized
  MISC_FEATURE (16)..(16): wherein TYR at position 16 is attached to polyethylene glycol
NFCCEFCCNPACTGCY

SEQ ID NO: 82 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein TYR at position 16 is attached to polyethylene glycol
NFCCEYCCNPACTGCY

SEQ ID NO: 83 - protein, 16 aa, Artificial Sequence
  Chemically Synthesized
  MISC_FEATURE (1)..(1): wherein ASN at position 1 is attached to polyethylene glycol
NFCCEYCCNPACTGCY

SEQ ID NO: 84 - protein, 16 aa, Artificial Sequence
  Chemically Synthesized
  MISC_FEATURE (16)..(16): wherein TYR at position 16 is attached to polyethylene glycol
NFCCEYCCNPACTGCY

SEQ ID NO: 85 - protein, 14 aa, Artificial Sequence
  Chemically Synthesized
CCESCCNPACTGCY

SEQ ID NO: 86 - protein, 14 aa, Artificial Sequence
  Chemically Synthesized
CCEFCCNPACTGCY

SEQ ID NO: 87 - protein, 13 aa, Artificial Sequence
  Chemically Synthesized
CCESCCNPACTGC

SEQ ID NO: 88 - protein, 13 aa, Artificial Sequence
  Chemically Synthesized
CCEFCCNPACTGC

SEQ ID NO: 89 - protein, 14 aa, Artificial Sequence
  Chemically Synthesized
  MISC_FEATURE (1)..(2): wherein x is penicillamine
  MISC_FEATURE (5)..(6): wherein x is penicillamine
  MISC_FEATURE (10)..(10): wherein x is penicillamine
  MISC_FEATURE (13)..(13): wherein x is penicillamine
XXEYXXNPAXTGXY

SEQ ID NO: 90 - protein, 13 aa, Artificial Sequence
  Chemically Synthesized
  MISC_FEATURE (1)..(2): wherein x is penicillamine
  MISC_FEATURE (5)..(6): wherein x is penicillamine
  MISC_FEATURE (10)..(10): wherein x is penicillamine
  MISC_FEATURE (13)..(13): wherein x is penicillamine
XXEYXXNPAXTGX

SEQ ID NO: 91 - protein, 22 aa, Artificial Sequence
  Chemically Synthesized
  MISC_FEATURE (1)..(6): x is natural, unnatural, analogue, L, D, or methylated amino acid or any combination
  MISC_FEATURE (11)..(11): x is natural, unnatural, analogue, L, D, or methylated amino acid or any combination
  MISC_FEATURE (15)..(17): x is natural, unnatural, analogue, L, D, or methylated amino acid or any combination
  MISC_FEATURE (19)..(20): x is natural, unnatural, analogue, L, D, or methylated amino acid or any combination
  MISC_FEATURE (22)..(22): x is natural, unnatural, analogue, L, D, or methylated amino acid or any combination
XXXXXXNYCCXYCCXXXCXXCX

SEQ ID NO: 92 - protein, 22 aa, Artificial Sequence
  Chemically Synthesized
  MISC_FEATURE (1)..(6): x is natural, unnatural, analogue, L, D, or methylated amino acid or any combination
  MISC_FEATURE (11)..(11): x is natural, unnatural, analogue, L, D, or methylated amino acid or any combination
  MISC_FEATURE (15)..(17): x is natural, unnatural, analogue, L, D, or methylated amino acid or any combination
  MISC_FEATURE (19)..(20): x is natural, unnatural, analogue, L, D, or methylated amino acid or any combination
  MISC_FEATURE (22)..(22): x is natural, unnatural, analogue, L, D, or methylated amino acid or any combination
XXXXXXNFCCXFCCXXXCXXCX

SEQ ID NO: 93 - protein, 16 aa, Artificial Sequence
  Chemically Synthesized
  MISC_FEATURE (5)..(5): x is natural, unnatural, analogue, L, D, or methylated amino acid or any combination
  MISC_FEATURE (9)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is natural, unnatural, analogue, L, D, or methylated amino acid or any combination
NFCCXFCCXXXCXXCX

SEQ ID NO: 94 - protein, 16 aa, Artificial Sequence
  Chemically Synthesized
  MISC_FEATURE (3)..(3): wherein x is penicillamine
  MISC_FEATURE (5)..(5): x is natural, unnatural, analogue, L, D, or methylated amino acid or any combination
  MISC_FEATURE (8)..(8): wherein x is penicillamine
  MISC_FEATURE (9)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is natural, unnatural, analogue, L, D, or methylated amino acid or any combination
NFXCXFCXXXXCXXCX

SEQ ID NO: 95 - protein, 16 aa, Artificial Sequence
  Chemically Synthesized
  MISC_FEATURE (3)..(4): wherein x is a cysteine, penicillamine homocysteine, or 3-mercaptoproline
  MISC_FEATURE (5)..(6): x is natural, unnatural, analogue, L, D, or methylated amino acid or any combination
  MISC_FEATURE (7)..(8): wherein x is a cysteine, penicillamine homocysteine, or 3-mercaptoproline
  MISC_FEATURE (9)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): wherein x is a cysteine, penicillamine homocysteine, or 3-mercaptoproline
  MISC_FEATURE (16)..(16): x is natural, unnatural, analogue, L, D, or methylated amino acid or any combination
NFXXXXXXXXXXXXXX

SEQ ID NO: 96 - protein, 14 aa, Artificial Sequence
  Chemically Synthesized
  MISC_FEATURE (1)..(2): wherein x is a cysteine, penicillamine homocysteine, or 3-mercaptoproline
  MISC_FEATURE (4)..(4): x is natural, unnatural, analogue, L, D, or methylated amino acid or any combination
  MISC_FEATURE (5)..(6): wherein x is a cysteine, penicillamine homocysteine, or 3-mercaptoproline
  MISC_FEATURE (10)..(10): wherein x is a cysteine, penicillamine homocysteine, or 3-mercaptoproline
  MISC_FEATURE (13)..(13): wherein x is a cysteine, penicillamine homocysteine, or 3-mercaptoproline
XXEXXXNPAXTGXY

SEQ ID NO: 97 - protein, 13 aa, Artificial Sequence
  Chemically Synthesized
  MISC_FEATURE (1)..(2): wherein x is a cysteine, penicillamine homocysteine, or 3-mercaptoproline
  MISC_FEATURE (4)..(4): x is natural, unnatural, analogue, L, D, or methylated amino acid or any combination
  MISC_FEATURE (5)..(6): wherein x is a cysteine, penicillamine homocysteine, or 3-mercaptoproline
  MISC_FEATURE (10)..(10): wherein x is a cysteine, penicillamine homocysteine, or 3-mercaptoproline
  MISC_FEATURE (13)..(13): wherein x is a cysteine, penicillamine homocysteine, or 3-mercaptoproline
XXEXXXNPAXTGX

SEQ ID NO: 98 - protein, 20 aa, Artificial Sequence
  Chemically Synthesized
  MISC_FEATURE (1)..(1): x is none or a natural, unnatural, analogue, L, D, or methylated amino acid or any combination
  MISC_FEATURE (2)..(2): x is none or a natural, unnatural, analogue, L, D, or methylated amino acid or any combination
  MISC_FEATURE (3)..(3): x is none or a natural, unnatural, analogue, L, D, or methylated amino acid or any combination
  MISC_FEATURE (4)..(4): x is none or a natural, unnatural, analogue, L, D, or methylated amino acid or any combination
  MISC_FEATURE (5)..(5): x is none or a natural, unnatural, analogue, L, D, or methylated amino acid or any combination
  MISC_FEATURE (6)..(6): x is none or a natural, unnatural, analogue, L, D, or methylated amino acid or any combination
  MISC_FEATURE (7)..(8): wherein x is a cysteine, penicillamine homocysteine, or 3-mercaptoproline
  MISC_FEATURE (9)..(10): x is natural, unnatural, analogue, L, D, or methylated amino acid or any combination
  MISC_FEATURE (11)..(12): wherein x is a cysteine, penicillamine homocysteine, or 3-mercaptoproline
  MISC_FEATURE (13)..(15): x is natural, unnatural, analogue, L, D, or methylated amino acid or any combination
  MISC_FEATURE (16)..(16): wherein x is a cysteine, penicillamine homocysteine, or 3-mercaptoproline
  MISC_FEATURE (17)..(18): x is natural, unnatural, analogue, L, D, or methylated amino acid or any combination
  MISC_FEATURE (19)..(19): wherein x is a cysteine, penicillamine homocysteine, or 3-mercaptoproline
  MISC_FEATURE (20)..(20): x is none or a natural, unnatural, analogue, L, D, or methylated amino acid or any combination
XXXXXXXXXXXXXXXXXXXX

SEQ ID NO: 99 - protein, 16 aa, Artificial Sequence
  Chemically Synthesized
  MISC_FEATURE (1)..(1): wherein ASN is a D amino acid
  MISC_FEATURE (16)..(16): wherein LEU is a D amino acid
  MISC_FEATURE (16)..(16): wherein LEU is conjugated to an AMIDE
NDECELCVNVACTGCL

SEQ ID NO: 100 - protein, 16 aa, Artificial Sequence
  Chemically Synthesized
  MISC_FEATURE (1)..(1): wherein ASN is a D amino acid
  MISC_FEATURE (16)..(16): wherein SER is a D amino acid
NDECELCVNVACTGCS

SEQ ID NO: 101 - protein, 16 aa, Artificial Sequence
  Chemically Synthesized
  MISC_FEATURE (1)..(1): wherein ASN is a D amino acid
  MISC_FEATURE (16)..(16): wherein SER is a D amino acid
  MISC_FEATURE (16)..(16): wherein SER is conjugated to an AMIDE
NDECELCVNVACTGCS

SEQ ID NO: 102 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NDECELCVNVACTGCY

SEQ ID NO: 103 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
  MISC_FEATURE (16)..(16): wherein TYR is conjugated to an AMIDE
NDECELCVNVACTGCY

SEQ ID NO: 104 - protein, 16 aa, Artificial Sequence
  Chemically Synthesized
  MISC_FEATURE (1)..(1): wherein GLU is conjugated to Py
  MISC_FEATURE (16)..(16): wherein LEU is conjugated to an AMIDE
  MISC_FEATURE (16)..(16): wherein LEU is a D amino acid
EDECELCVNVACTGCL

SEQ ID NO: 105 - protein, 16 aa, Artificial Sequence
  Chemically Synthesized
  MISC_FEATURE (1)..(1): wherein ASN is attached to polyethylene glycol
  MISC_FEATURE (16)..(16): wherein LEU is attached to polyethylene glycol
NDECELCVNVACTGCL

SEQ ID NO: 106 - protein, 16 aa, Artificial Sequence
  Chemically Synthesized
  MISC_FEATURE (1)..(1): wherein ASN is attached to polyethylene glycol
NDECELCVNVACTGCL

SEQ ID NO: 107 - protein, 16 aa, Artificial Sequence
  Chemically Synthesized
  MISC_FEATURE (16)..(16): wherein LEU is attached to polyethylene glycol
NDECELCVNVACTGCL

SEQ ID NO: 108 - protein, 16 aa, Artificial Sequence
  Chemically Synthesized
  MISC_FEATURE (1)..(3): x is natural, unnatural, analogue, L, D, or methylated amino acid or any combination
  MISC_FEATURE (4)..(4): wherein x is a cysteine, penicillamine homocysteine, or 3-mercaptoproline
  MISC_FEATURE (5)..(6): x is natural, unnatural, analogue, L, D, or methylated amino acid or any combination
  MISC_FEATURE (7)..(7): wherein x is a cysteine, penicillamine homocysteine, or 3-mercaptoproline
  MISC_FEATURE (8)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): wherein x is a cysteine, penicillamine homocysteine, or 3-mercaptoproline
  MISC_FEATURE (16)..(16): x is natural, unnatural, analogue, L, D, or methylated amino acid or any combination
XXXXXXXXXXXXXXXX

SEQ ID NO: 109 - protein, 16 aa, Artificial Sequence
  Chemically Synthesized
NDDCELCVNVACTGCL

SEQ ID NO: 110 - protein, 16 aa, Artificial Sequence
  Chemically Synthesized
EDDCELCVNVACTGCL

SEQ ID NO: 111 - protein, 16 aa, Artificial Sequence
  Chemically Synthesized
EDECELCVNVACTGCL

SEQ ID NO: 112 - protein, 16 aa, Artificial Sequence
  Chemically Synthesized
EEDCELCVNVACTGCL

SEQ ID NO: 113 - protein, 16 aa, Artificial Sequence
  Chemically Synthesized
EEECELCVNVACTGCL

SEQ ID NO: 114 - protein, 16 aa, Artificial Sequence
  Chemically Synthesized
DDDCELCVNVACTGCL

SEQ ID NO: 115 - protein, 16 aa, Artificial Sequence
  Chemically Synthesized
DDECELCVNVACTGCL

SEQ ID NO: 116 - protein, 16 aa, Artificial Sequence
  Chemically Synthesized
DEDCELCVNVACTGCL

SEQ ID NO: 117 - protein, 16 aa, Artificial Sequence
  Chemically Synthesized
DEECELCVNVACTGCL

SEQ ID NO: 118 - protein, 16 aa, Artificial Sequence
  Chemically Synthesized
QDDCELCVNVACTGCL

SEQ ID NO: 119 - protein, 16 aa, Artificial Sequence
  Chemically Synthesized
QDECELCVNVACTGCL

SEQ ID NO: 120 - protein, 16 aa, Artificial Sequence
  Chemically Synthesized
QEDCELCVNVACTGCL

SEQ ID NO: 121 - protein, 16 aa, Artificial Sequence
  Chemically Synthesized
QEECELCVNVACTGCL

SEQ ID NO: 122 - protein, 16 aa, Artificial Sequence
  Chemically Synthesized
KDDCELCVNVACTGCL

SEQ ID NO: 123 - protein, 16 aa, Artificial Sequence
  Chemically Synthesized
KDECELCVNVACTGCL

SEQ ID NO: 124 - protein, 16 aa, Artificial Sequence
  Chemically Synthesized
KEDCELCVNVACTGCL

SEQ ID NO: 125 - protein, 16 aa, Artificial Sequence
  Chemically Synthesized
KEECELCVNVACTGCL

SEQ ID NO: 126 - protein, 16 aa, Artificial Sequence
  Chemically Synthesized
EDDCELCVNVACTGCS

SEQ ID NO: 127 - protein, 16 aa, Artificial Sequence
  Chemically Synthesized
EDECELCVNVACTGCS

SEQ ID NO: 128 - protein, 16 aa, Artificial Sequence
  Chemically Synthesized
EEDCELCVNVACTGCS

SEQ ID NO: 129 - protein, 16 aa, Artificial Sequence
  Chemically Synthesized
EEECELCVNVACTGCS

SEQ ID NO: 130 - protein, 16 aa, Artificial Sequence
  Chemically Synthesized
DDDCELCVNVACTGCS

SEQ ID NO: 131 - protein, 16 aa, Artificial Sequence
  Chemically Synthesized
DDECELCVNVACTGCS

SEQ ID NO: 132 - protein, 16 aa, Artificial Sequence
  Chemically Synthesized
DEDCELCVNVACTGCS

SEQ ID NO: 133 - protein, 16 aa, Artificial Sequence
  Chemically Synthesized
DEECELCVNVACTGCS

SEQ ID NO: 134 - protein, 16 aa, Artificial Sequence
  Chemically Synthesized
QDDCELCVNVACTGCS

SEQ ID NO: 135 - protein, 16 aa, Artificial Sequence
  Chemically Synthesized
QDECELCVNVACTGCS

SEQ ID NO: 136 - protein, 16 aa, Artificial Sequence
  Chemically Synthesized
QEDCELCVNVACTGCS

SEQ ID NO: 137 - protein, 16 aa, Artificial Sequence
  Chemically Synthesized
QEECELCVNVACTGCS

SEQ ID NO: 138 - protein, 16 aa, Artificial Sequence
  Chemically Synthesized
KDDCELCVNVACTGCS

SEQ ID NO: 139 - protein, 16 aa, Artificial Sequence
  Chemically Synthesized
KDECELCVNVACTGCS

SEQ ID NO: 140 - protein, 16 aa, Artificial Sequence
  Chemically Synthesized
KEDCELCVNVACTGCS

SEQ ID NO: 141 - protein, 16 aa, Artificial Sequence
  Chemically Synthesized
KEECELCVNVACTGCS

SEQ ID NO: 142 - protein, 16 aa, Artificial Sequence
  Chemically Synthesized
EDDCELCINMACTGCL

SEQ ID NO: 143 - protein, 16 aa, Artificial Sequence
  Chemically Synthesized
EDECELCINMACTGCL

SEQ ID NO: 144 - protein, 16 aa, Artificial Sequence
  Chemically Synthesized
EEDCELCINMACTGCL

SEQ ID NO: 145 - protein, 16 aa, Artificial Sequence
  Chemically Synthesized
EEECELCINMACTGCL

SEQ ID NO: 146 - protein, 16 aa, Artificial Sequence
  Chemically Synthesized
DDDCELCINMACTGCL

SEQ ID NO: 147 - protein, 16 aa, Artificial Sequence
  Chemically Synthesized
DDECELCINMACTGCL

SEQ ID NO: 148 - protein, 16 aa, Artificial Sequence
  Chemically Synthesized
DEDCELCINMACTGCL

SEQ ID NO: 149 - protein, 16 aa, Artificial Sequence
  Chemically Synthesized
DEECELCINMACTGCL

SEQ ID NO: 150 - protein, 16 aa, Artificial Sequence
  Chemically Synthesized
QDDCELCINMACTGCL

SEQ ID NO: 151 - protein, 16 aa, Artificial Sequence
  Chemically Synthesized
QDECELCINMACTGCL

SEQ ID NO: 152 - protein, 16 aa, Artificial Sequence
  Chemically Synthesized
QEDCELCINMACTGCL

SEQ ID NO: 153 - protein, 16 aa, Artificial Sequence
  Chemically Synthesized
QEECELCINMACTGCL

SEQ ID NO: 154 - protein, 16 aa, Artificial Sequence
  Chemically Synthesized
KDDCELCINMACTGCL

SEQ ID NO: 155 - protein, 16 aa, Artificial Sequence
  Chemically Synthesized
KDECELCINMACTGCL

SEQ ID NO: 156 - protein, 16 aa, Artificial Sequence
  Chemically Synthesized
KEDCELCINMACTGCL

SEQ ID NO: 157 - protein, 16 aa, Artificial Sequence
  Chemically Synthesized
KEECELCINMACTGCL

SEQ ID NO: 158 - protein, 16 aa, Artificial Sequence
  Chemically Synthesized
EDDCELCINMACTGCS

SEQ ID NO: 159 - protein, 16 aa, Artificial Sequence
  Chemically Synthesized
EDECELCINMACTGCS

SEQ ID NO: 160 - protein, 16 aa, Artificial Sequence
  Chemically Synthesized
EEDCELCINMACTGCS

SEQ ID NO: 161 - protein, 16 aa, Artificial Sequence
  Chemically Synthesized
EEECELCINMACTGCS

SEQ ID NO: 162 - protein, 16 aa, Artificial Sequence
  Chemically Synthesized
DDDCELCINMACTGCS

SEQ ID NO: 163 - protein, 16 aa, Artificial Sequence
  Chemically Synthesized
DDECELCINMACTGCS

SEQ ID NO: 164 - protein, 16 aa, Artificial Sequence
  Chemically Synthesized
DEDCELCINMACTGCS

SEQ ID NO: 165 - protein, 16 aa, Artificial Sequence
  Chemically Synthesized
DEECELCINMACTGCS

SEQ ID NO: 166 - protein, 16 aa, Artificial Sequence
  Chemically Synthesized
QDDCELCINMACTGCS

SEQ ID NO: 167 - protein, 16 aa, Artificial Sequence
  Chemically Synthesized
QDECELCINMACTGCS

SEQ ID NO: 168 - protein, 16 aa, Artificial Sequence
  Chemically Synthesized
QEDCELCINMACTGCS

SEQ ID NO: 169 - protein, 16 aa, Artificial Sequence
  Chemically Synthesized
QEECELCINMACTGCS

SEQ ID NO: 170 - protein, 16 aa, Artificial Sequence
  Chemically Synthesized
KDDCELCINMACTGCS

SEQ ID NO: 171 - protein, 16 aa, Artificial Sequence
  Chemically Synthesized
KDECELCINMACTGCS

SEQ ID NO: 172 - protein, 16 aa, Artificial Sequence
  Chemically Synthesized
KEDCELCINMACTGCS

SEQ ID NO: 173 - protein, 16 aa, Artificial Sequence
  Chemically Synthesized
KEECELCINMACTGCS

SEQ ID NO: 174 - protein, 15 aa, Artificial Sequence
  Chemically Synthesized
  MISC_FEATURE (1)..(3): x is natural, unnatural, analogue, L, D, or methylated amino acid or any combination
  MISC_FEATURE (4)..(4): wherein x is a cysteine, penicillamine homocysteine, or 3-mercaptoproline
  MISC_FEATURE (5)..(6): x is natural, unnatural, analogue, L, D, or methylated amino acid or any combination
  MISC_FEATURE (7)..(7): wherein x is a cysteine, penicillamine homocysteine, or 3-mercaptoproline
  MISC_FEATURE (8)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): wherein x is a cysteine, penicillamine homocysteine, or 3-mercaptoproline
XXXXXXXXXXXXXXX

SEQ ID NO: 175 - protein, 15 aa, Artificial Sequence
  Chemically Synthesized
SHTCEICAFAACAGC

SEQ ID NO: 176 - protein, 15 aa, Artificial Sequence
  Chemically Synthesized
SHTCEICANAACAGC

SEQ ID NO: 177 - protein, 15 aa, Artificial Sequence
  Chemically Synthesized
SHTCELCANAACAGC

SEQ ID NO: 178 - protein, 15 aa, Artificial Sequence
  Chemically Synthesized
SHTCEVCANAACAGC

SEQ ID NO: 179 - protein, 15 aa, Artificial Sequence
  Chemically Synthesized
SHTCEYCANAACAGC

SEQ ID NO: 180 - protein, 15 aa, Artificial Sequence
  Chemically Synthesized
SHTCEICANAACAGC

SEQ ID NO: 181 - protein, 15 aa, Artificial Sequence
  Chemically Synthesized
SHTCELCANAACAGC

SEQ ID NO: 182 - protein, 15 aa, Artificial Sequence
  Chemically Synthesized
SHTCEVCANAACAGC

SEQ ID NO: 183 - protein, 15 aa, Artificial Sequence
  Chemically Synthesized
SHTCEYCANAACAGC

SEQ ID NO: 184 - protein, 15 aa, Artificial Sequence
  Chemically Synthesized
SHTCEICANAACAGC

SEQ ID NO: 185 - protein, 15 aa, Artificial Sequence
  Chemically Synthesized
SHTCELCANAACAGC

SEQ ID NO: 186 - protein, 15 aa, Artificial Sequence
  Chemically Synthesized
SHTCEVCANAACAGC

SEQ ID NO: 187 - protein, 15 aa, Artificial Sequence
  Chemically Synthesized
SHTCEYCANAACAGC

SEQ ID NO: 188 - protein, 15 aa, Artificial Sequence
  Chemically Synthesized
SHTCEICANAACAGC

SEQ ID NO: 189 - protein, 15 aa, Artificial Sequence
  Chemically Synthesized
SHTCELCANAACAGC

SEQ ID NO: 190 - protein, 15 aa, Artificial Sequence
  Chemically Synthesized
SHTCEVCANAACAGC

SEQ ID NO: 191 - protein, 15 aa, Artificial Sequence
  Chemically Synthesized
SHTCEYCANAACAGC

SEQ ID NO: 192 - protein, 15 aa, Artificial Sequence
  Chemically Synthesized
NDECEICANAACAGC

SEQ ID NO: 193 - protein, 15 aa, Artificial Sequence
  Chemically Synthesized
NDECELCANAACAGC

SEQ ID NO: 194 - protein, 15 aa, Artificial Sequence
  Chemically Synthesized
NDECEVCANAACAGC

SEQ ID NO: 195 - protein, 15 aa, Artificial Sequence
  Chemically Synthesized
NDECEYCANAACAGC

SEQ ID NO: 196 - protein, 15 aa, Artificial Sequence
  Chemically Synthesized
NDECEICANAACAGC

SEQ ID NO: 197 - protein, 15 aa, Artificial Sequence
  Chemically Synthesized
NDECELCANAACAGC

SEQ ID NO: 198 - protein, 15 aa, Artificial Sequence
  Chemically Synthesized
NDECEVCANAACAGC

SEQ ID NO: 199 - protein, 15 aa, Artificial Sequence
  Chemically Synthesized
NDECEYCANAACAGC

SEQ ID NO: 200 - protein, 15 aa, Artificial Sequence
  Chemically Synthesized
NDECEICANAACAGC

SEQ ID NO: 201 - protein, 15 aa, Artificial Sequence
  Chemically Synthesized
NDECELCANAACAGC

SEQ ID NO: 202 - protein, 15 aa, Artificial Sequence
  Chemically Synthesized
NDECEVCANAACAGC

SEQ ID NO: 203 - protein, 15 aa, Artificial Sequence
  Chemically Synthesized
NDECEYCANAACAGC

SEQ ID NO: 204 - protein, 15 aa, Artificial Sequence
  Chemically Synthesized
NDECEICANAACAGC

SEQ ID NO: 205 - protein, 15 aa, Artificial Sequence
  Chemically Synthesized
NDECELCANAACAGC

SEQ ID NO: 206 - protein, 15 aa, Artificial Sequence
  Chemically Synthesized
NDECEVCANAACAGC

SEQ ID NO: 207 - protein, 15 aa, Artificial Sequence
  Chemically Synthesized
NDECEYCANAACAGC

SEQ ID NO: 208 - protein, 17 aa, Artificial Sequence
  Chemically Synthesized
  MISC_FEATURE (1)..(3): x is natural, unnatural, analogue, L, D, or methylated amino acid or any combination
  MISC_FEATURE (4)..(4): wherein x is a cysteine, penicillamine homocysteine, or 3-mercaptoproline
  MISC_FEATURE (5)..(6): x is natural, unnatural, analogue, L, D, or methylated amino acid or any combination
  MISC_FEATURE (7)..(7): wherein x is a cysteine, penicillamine homocysteine, or 3-mercaptoproline
  MISC_FEATURE (8)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): x is none or a natural, unnatural, analogue, L, D, or methylated amino acid or any combination
  MISC_FEATURE (16)..(16): x is none or a natural, unnatural, analogue, L, D, or methylated amino acid or any combination
  MISC_FEATURE (17)..(17): x is any natural, unnatural, analogue, L, D, or methylated amino acid or any combination
XXXXXXXXXXXXXXXXX

SEQ ID NO: 209 - protein, 15 aa, Artificial Sequence
  Chemically Synthesized
QEECELCINMACTGY

SEQ ID NO: 210 - protein, 15 aa, Artificial Sequence
  Chemically Synthesized
QEECETCINMACTGY

SEQ ID NO: 211 - protein, 15 aa, Artificial Sequence
  Chemically Synthesized
QDECETCINMACTGY

SEQ ID NO: 212 - protein, 15 aa, Artificial Sequence
  Chemically Synthesized
QDDCETCINMACTGY

SEQ ID NO: 213 - protein, 15 aa, Artificial Sequence
  Chemically Synthesized
QEDCETCINMACTGY

SEQ ID NO: 214 - protein, 15 aa, Artificial Sequence
  Chemically Synthesized
QEECEECINMACTGY

SEQ ID NO: 215 - protein, 15 aa, Artificial Sequence
  Chemically Synthesized
QDECEECINMACTGY

SEQ ID NO: 216 - protein, 15 aa, Artificial Sequence
  Chemically Synthesized
QDDCEECINMACTGY

SEQ ID NO: 217 - protein, 15 aa, Artificial Sequence
  Chemically Synthesized
QEDCEECINMACTGY

SEQ ID NO: 218 - protein, 15 aa, Artificial Sequence
  Chemically Synthesized
QEECEYCINMACTGY

SEQ ID NO: 219 - protein, 15 aa, Artificial Sequence
  Chemically Synthesized
QDECEYCINMACTGY

SEQ ID NO: 220 - protein, 15 aa, Artificial Sequence
  Chemically Synthesized
QDDCEYCINMACTGY

SEQ ID NO: 221 - protein, 15 aa, Artificial Sequence
  Chemically Synthesized
QEDCEYCINMACTGY

SEQ ID NO: 222 - protein, 15 aa, Artificial Sequence
  Chemically Synthesized
QEECEICINMACTGY

SEQ ID NO: 223 - protein, 15 aa, Artificial Sequence
  Chemically Synthesized
QDECEICINMACTGY

SEQ ID NO: 224 - protein, 15 aa, Artificial Sequence
  Chemically Synthesized
QDDCEICINMACTGY

SEQ ID NO: 225 - protein, 15 aa, Artificial Sequence
  Chemically Synthesized
QEDCEICINMACTGY

SEQ ID NO: 226 - protein, 16 aa, Artificial Sequence
  Chemically Synthesized
QEECETCINMACTGCS

SEQ ID NO: 227 - protein, 16 aa, Artificial Sequence
  Chemically Synthesized
QDECETCINMACTGCS

SEQ ID NO: 228 - protein, 16 aa, Artificial Sequence
  Chemically Synthesized
QDDCETCINMACTGCS

SEQ ID NO: 229 - protein, 16 aa, Artificial Sequence
  Chemically Synthesized
QEDCETCINMACTGCS

SEQ ID NO: 230 - protein, 16 aa, Artificial Sequence
  Chemically Synthesized
QEECEECINMACTGCS

SEQ ID NO: 231 - protein, 16 aa, Artificial Sequence
  Chemically Synthesized
QDECEECINMACTGCS

SEQ ID NO: 232 - protein, 16 aa, Artificial Sequence
  Chemically Synthesized
QDDCEECINMACTGCS

SEQ ID NO: 233 - protein, 16 aa, Artificial Sequence
  Chemically Synthesized
QEDCEECINMACTGCS

SEQ ID NO: 234 - protein, 16 aa, Artificial Sequence
  Chemically Synthesized
QEECEYCINMACTGCS

SEQ ID NO: 235 - protein, 16 aa, Artificial Sequence
  Chemically Synthesized
QDECEYCINMACTGCS

SEQ ID NO: 236 - protein, 16 aa, Artificial Sequence
  Chemically Synthesized
QDDCEYCINMACTGCS

SEQ ID NO: 237 - protein, 16 aa, Artificial Sequence
  Chemically Synthesized
QEDCEYCINMACTGCS

SEQ ID NO: 238 - protein, 16 aa, Artificial Sequence
  Chemically Synthesized
QEECEICINMACTGCS

SEQ ID NO: 239 - protein, 16 aa, Artificial Sequence
  Chemically Synthesized
QDECEICINMACTGCS

SEQ ID NO: 240 - protein, 16 aa, Artificial Sequence
  Chemically Synthesized
QDDCEICINMACTGCS

SEQ ID NO: 241 - protein, 16 aa, Artificial Sequence
  Chemically Synthesized
QEDCEICINMACTGCS

SEQ ID NO: 242 - protein, 22 aa, Artificial Sequence
  Chemically Synthesized
NSSNSSNYCCEKCCNPACTGCY

SEQ ID NO: 243 - protein, 16 aa, Artificial Sequence
  Chemically Synthesized
  MISC_FEATURE (1)..(1): wherein ASN is attached to polyethylene glycol
  MISC_FEATURE (16)..(16): wherein TYR is attached to polyethylene glycol
NFCCETCCNPACTGCY

SEQ ID NO: 244 - protein, 16 aa, Artificial Sequence
  Chemically Synthesized
  MISC_FEATURE (1)..(1): wherein ASN is attached to polyethylene glycol
NFCCETCCNPACTGCY

SEQ ID NO: 245 - protein, 16 aa, Artificial Sequence
  Chemically Synthesized
  MISC_FEATURE (16)..(16): wherein TYR is attached to polyethylene glycol
NFCCETCCNPACTGCY

SEQ ID NO: 246 - protein, 16 aa, Artificial Sequence
  Chemically Synthesized
NFCCEYCCNPACTGCY

SEQ ID NO: 247 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NFCCEYCCNPACTGCY

SEQ ID NO: 248 - protein, 16 aa, Artificial Sequence
  Chemically Synthesized
  MISC_FEATURE (16)..(16): wherein TYR is a D amino acid
NFCCEYCCNPACTGCY

SEQ ID NO: 249 - protein, 16 aa, Artificial Sequence
  Chemically Synthesized
  MISC_FEATURE (1)..(1): wherein ASN is a D amino acid
NFCCEYCCNPACTGCY

SEQ ID NO: 250 - protein, 16 aa, Artificial Sequence
  Synthesized peptide
  MISC_FEATURE (1)..(2): wherein Xaa is absent or any natural, unnatural amino acid or analogue thereof
  MISC_FEATURE (3)..(3): wherein Xaa is any natural, unnatural amino acid or analogue thereof
  MISC_FEATURE (5)..(11): wherein Xaa is any natural, unnatural amino acid or analogue thereof
  MISC_FEATURE (13)..(15): wherein Xaa is any natural, unnatural amino acid or analogue thereof
  MISC_FEATURE (16)..(16): wherein Xaa is absent or any natural, unnatural amino acid or analogue thereof
XXXCXXXXXXXCXXXX

SEQ ID NO: 251 - protein, 16 aa, Artificial Sequence
  Synthesized peptide
  MISC_FEATURE (1)..(1): wherein Xaa is a pyroglutamic acid
XDECELCVNVACTGCL

SEQ ID NO: 252 - protein, 15 aa, Artificial Sequence
  Synthesized peptide
PGTCEICAYAACTGC

SEQ ID NO: 253 - protein, 16 aa, Artificial Sequence
  Chemically synthesized peptide
  misc_feature (3)..(3): Xaa is alpha-aminoadipic acid
  misc_feature (16)..(16): Leu is a D amino acid
NDXCELCVNVACTGCL

SEQ ID NO: 254 - protein, 16 aa, Artificial Sequence
  Chemically synthesized peptide
  misc_feature (1)..(1): Glu is conjugated to Py
  misc_feature (3)..(3): Xaa is alpha-aminoadipic acid
  misc_feature (16)..(16): Leu is a D amino acid
  misc_feature (16)..(16): Leu is conjugated to an amide
EDXCELCVNVACTGCL

SEQ ID NO: 255 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Ser is a D amino acid
NDXCELCVNVACTGCS

SEQ ID NO: 256 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Tyr is a D amino acid
NDXCELCVNVACTGCY

SEQ ID NO: 257 - protein, 16 aa, Artificial Sequence
  Chemically synthesized peptide
  misc_feature (1)..(1): Xaa is none or a natural, unnatural or an analogue amino acid, may be L, D, or a methylated amino acid, or any combination
  misc_feature (2)..(2): Xaa is none or a natural, unnatural or an analogue amino acid, may be L, D, or a methylated amino acid, or any combination
  misc_feature (3)..(3): Xaa is any natural, unnatural or an analogue amino acid, may be L, D, or a methylated amino acid, or any combination
  misc_feature (5)..(11): Xaa is a natural, unnatural, analogue, L, D, or methylated amino acid or any combination
  misc_feature (13)..(15): Xaa is a natural, unnatural, analogue, L, D, or methylated amino acid or any combination
  misc_feature (16)..(16): Xaa is none or one natural, unnatural or an analogue amino acid, may be L, D, or a methylated amino acid, or any combination
XXXCXXXXXXXCXXXX

SEQ ID NO: 258 - protein, 16 aa, Artificial Sequence
  Chemically synthesized peptide
  misc_feature (3)..(3): Xaa is alpha-aminoadipic acid
NDXCELCVNVACTGCL

SEQ ID NO: 259 - protein, 15 aa, Artificial Sequence
  Chemically synthesized peptide
  misc_feature (2)..(2): Xaa is alpha-aminoadipic acid
DXCELCVNVACTGCL

SEQ ID NO: 260 - protein, 14 aa, Artificial Sequence
  Chemically synthesized peptide
  misc_feature (2)..(2): Xaa is alpha-aminoadipic acid
DXCELCVNVACTGC

SEQ ID NO: 261 - protein, 14 aa, Artificial Sequence
  Chemically synthesized peptide
  misc_feature (1)..(1): Xaa is alpha-aminoadipic acid
XCELCVNVACTGCL

SEQ ID NO: 262 - protein, 13 aa, Artificial Sequence
  Chemically synthesized peptide
  misc_feature (1)..(1): Xaa is alpha-aminoadipic acid
XCELCVNVACTGC

SEQ ID NO: 263 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Leu is a D amino acid
NDXCELCVNVACTGCL

SEQ ID NO: 264 - protein, 16 aa, Artificial Sequence
  Chemically synthesized peptide
  misc_feature (1)..(1): Asn is a D amino acid
  misc_feature (1)..(1): Asp is a D amino acid
  misc_feature (3)..(3): Xaa is alpha-aminoadipic acid
  misc_feature (16)..(16): Leu is a D amino acid
NDXCELCVNVACTGCL

SEQ ID NO: 265 - protein, 16 aa, Artificial Sequence
  Chemically synthesized peptide
  misc_feature (1)..(1): Asn is a D amino acid
  misc_feature (3)..(3): Xaa is alpha-aminoadipic acid
NDXCELCVNVACTGCL

SEQ ID NO: 266 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (6)..(6): Leu is a D amino acid
  misc_feature (16)..(16): Leu is a D amino acid
NDXCELCVNVACTGCL

SEQ ID NO: 267 - protein, 15 aa, Artificial Sequence
  Chemically synthesized peptide
  misc_feature (3)..(3): Xaa is alpha-aminoadipic acid
NDXCELCVNVACTGC

SEQ ID NO: 268 - protein, 16 aa, Artificial Sequence
  Chemically synthesized peptide
  misc_feature (1)..(1): Asn is attached to polyethylene glycol
  misc_feature (3)..(3): Xaa is alpha-aminoadipic acid
  misc_feature (16)..(16): Leu is a D amino acid
  misc_feature (16)..(16): Leu is attached to polyethylene glycol
NDXCELCVNVACTGCL

SEQ ID NO: 269 - protein, 16 aa, Artificial Sequence
  Chemically synthesized peptide
  misc_feature (1)..(1): Asn is attached to polyethylene glycol
  misc_feature (1)..(1): Asn is a D amino acid
  misc_feature (3)..(3): Xaa is alpha-aminoadipic acid
  misc_feature (16)..(16): Leu is a D amino acid
  misc_feature (16)..(16): Leu is attached to polyethylene glycol
NDXCELCVNVACTGCL

SEQ ID NO: 270 - protein, 16 aa, Artificial Sequence
  Chemically synthesized peptide
  misc_feature (1)..(1): Asn is attached to polyethylene glycol
  misc_feature (1)..(1): Asn is a D amino acid
  misc_feature (2)..(2): Asp is a D amino acid
  misc_feature (3)..(3): Xaa is alpha-aminoadipic acid
  misc_feature (16)..(16): Leu is a D amino acid
  misc_feature (16)..(16): Leu is attached to polyethylene glycol
NDXCELCVNVACTGCL

SEQ ID NO: 271 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Leu is a D amino acid
  misc_feature (16)..(16): Leu is attached to polyethylene glycol
NDXCELCVNVACTGCL

SEQ ID NO: 272 - protein, 16 aa, Artificial Sequence
  Chemically synthesized peptide
  misc_feature (1)..(1): Asn is attached to polyethylene glycol
  misc_feature (3)..(3): Xaa is alpha-aminoadipic acid
  misc_feature (16)..(16): Leu is a D amino acid
NDXCELCVNVACTGCL

SEQ ID NO: 273 - protein, 16 aa, Artificial Sequence
  Chemically synthesized peptide
  misc_feature (1)..(1): Asn is attached to polyethylene glycol
  misc_feature (1)..(1): Asn is a D amino acid
  misc_feature (3)..(3): Xaa is alpha-aminoadipic acid
  misc_feature (16)..(16): Leu is a D amino acid
NDXCELCVNVACTGCL

SEQ ID NO: 274 - protein, 16 aa, Artificial Sequence
  Chemically synthesized peptide
  misc_feature (3)..(3): Xaa is alpha-aminoadipic acid
  misc_feature (16)..(16): Leu is a D amino acid
  misc_feature (16)..(16): Leu is attached to polyethylene glycol
NDXCELCVNVACTGCL

SEQ ID NO: 275 - protein, 16 aa, Artificial Sequence
  Chemically synthesized peptide
  misc_feature (1)..(1): Asn is attached to polyethylene glycol
  misc_feature (1)..(1): Asn is a D amino acid
  misc_feature (2)..(2): Asp is a D amino acid
  misc_feature (3)..(3): Xaa is alpha-aminoadipic acid
  misc_feature (16)..(16): Leu is a D amino acid
NDXCELCVNVACTGCL

SEQ ID NO: 276 - protein, 16 aa, Artificial Sequence
  Chemically synthesized peptide
  misc_feature (1)..(1): Asn is a D amino acid
  misc_feature (2)..(2): Asp is a D amino acid
  misc_feature (3)..(3): Xaa is alpha-aminoadipic acid
  misc_feature (16)..(16): Leu is a D amino acid
  misc_feature (16)..(16): Leu is attached to polyethylene glycol
NDXCELCVNVACTGCL

SEQ ID NO: 277 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Xaa is 3-(2-naphthyl)alanine
NDXCELCVNVACTGCX

SEQ ID NO: 278 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (8)..(8): Xaa is alpha-amino isobutyric acid
  misc_feature (10)..(10): Xaa is alpha-amino isobutyric acid
  misc_feature (16)..(16): Leu is a D amino acid
NDXCELCXNXACTGCL

SEQ ID NO: 279 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (7)..(7): Asp is attached to a Lactam bridge
  misc_feature (15)..(15): Xaa is ornithine
  misc_feature (16)..(16): Leu is a D amino acid
NDXCELDVNVACTGXL

SEQ ID NO: 280 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Leu is a D amino acid
NDXCEYCVNVACTGCL

SEQ ID NO: 281 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Leu is a D amino acid
NDXCESCVNVACTGCL

SEQ ID NO: 282 - protein, 16 aa, Artificial Sequence
  Chemically synthesized peptide
  misc_feature (1)..(1): Asn is attached to polyethylene glycol
  misc_feature (1)..(1): Asn is a D amino acid
  misc_feature (3)..(3): Xaa is alpha-aminoadipic acid
  misc_feature (16)..(16): Leu is a D amino acid
  misc_feature (16)..(16): Leu is attached to polyethylene glycol
NDXCEYCVNVACTGCL

SEQ ID NO: 283 - protein, 16 aa, Artificial Sequence
  Chemically synthesized peptide
  misc_feature (1)..(1): Asn is attached to polyethylene glycol
  misc_feature (1)..(1): Asn is a D amino acid
  misc_feature (3)..(3): Xaa is alpha-aminoadipic acid
  misc_feature (16)..(16): Leu is a D amino acid
NDXCEYCVNVACTGCL

SEQ ID NO: 284 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Leu is a D amino acid
  misc_feature (16)..(16): Leu is attached to polyethylene glycol
NDXCEYCVNVACTGCL

SEQ ID NO: 285 - protein, 16 aa, Artificial Sequence
  Chemically synthesized peptide
  misc_feature (1)..(1): Asn is attached to polyethylene glycol
  misc_feature (1)..(1): Asn is a D amino acid
  misc_feature (3)..(3): Xaa is alpha-aminoadipic acid
  misc_feature (16)..(16): Leu is a D amino acid
  misc_feature (16)..(16): Leu is attached to polyethylene glycol
NDXCESCVNVACTGCL

SEQ ID NO: 286 - protein, 16 aa, Artificial Sequence
  Chemically synthesized peptide
  misc_feature (1)..(1): Asn is attached to polyethylene glycol
  misc_feature (1)..(1): Asn is a D amino acid
  misc_feature (3)..(3): Xaa is alpha-aminoadipic acid
  misc_feature (16)..(16): Leu is a D amino acid
NDXCESCVNVACTGCL

SEQ ID NO: 287 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Leu is a D amino acid
  misc_feature (16)..(16): Leu is attached to polyethylene glycol
NDXCESCVNVACTGCL

SEQ ID NO: 288 - protein, 16 aa, Artificial Sequence
  Chemically synthesized peptide
  misc_feature (3)..(3): Xaa is alpha-aminoadipic acid
NDXCELCVNVACTGCS

SEQ ID NO: 289 - protein, 16 aa, Artificial Sequence
  Chemically synthesized peptide
  misc_feature (1)..(1): Asn is attached to polyethylene glycol
  misc_feature (3)..(3): Xaa is alpha-aminoadipic acid
  misc_feature (16)..(16): Ser is attached to polyethylene glycol
NDXCELCVNVACTGCS

SEQ ID NO: 290 - protein, 16 aa, Artificial Sequence
  Chemically synthesized peptide
  misc_feature (1)..(1): Asn is attached to polyethylene glycol
  misc_feature (3)..(3): Xaa is alpha-aminoadipic acid
NDXCELCVNVACTGCS

SEQ ID NO: 291 - protein, 16 aa, Artificial Sequence
  Chemically synthesized peptide
  misc_feature (3)..(3): Xaa is alpha-aminoadipic acid
  misc_feature (16)..(16): Ser is attached to polyethylene glycol
NDXCELCVNVACTGCS

SEQ ID NO: 292 - protein, 16 aa, Artificial Sequence
  Chemically synthesized peptide
  misc_feature (1)..(1): Asn is attached to polyethylene glycol
  misc_feature (3)..(3): Xaa is alpha-aminoadipic acid
  misc_feature (16)..(16): Ser is a D amino acid
  misc_feature (16)..(16): Ser is attached to polyethylene glycol
NDXCELCVNVACTGCS

SEQ ID NO: 293 - protein, 16 aa, Artificial Sequence
  Chemically synthesized peptide
  misc_feature (1)..(1): Asn is attached to polyethylene glycol
  misc_feature (3)..(3): Xaa is alpha-aminoadipic acid
  misc_feature (16)..(16): Ser is a D amino acid
NDXCELCVNVACTGCS

SEQ ID NO: 294 - protein, 16 aa, Artificial Sequence
  Chemically synthesized peptide
  misc_feature (3)..(3): Xaa is alpha-aminoadipic acid
  misc_feature (16)..(16): Ser is a D amino acid
  misc_feature (16)..(16): Ser is attached to polyethylene glycol
NDXCELCVNVACTGCS

SEQ ID NO: 295 - protein, 16 aa, Artificial Sequence
  Chemically synthesized peptide
  misc_feature (3)..(3): Xaa is alpha-aminoadipic acid
  misc_feature (5)..(6): Xaa is a natural, unnatural, analogue, L, D, or methylated amino acid or any combination
  misc_feature (8)..(11): Xaa is a natural, unnatural, analogue, L, D, or methylated amino acid or any combination
  misc_feature (13)..(14): Xaa is a natural, unnatural, analogue, L, D, or methylated amino acid or any combination
  misc_feature (16)..(16): Xaa is a natural, unnatural, analogue, L, D, or methylated amino acid or any combination
NDXCXXCXXXXCXXCX

SEQ ID NO: 296 - protein, 16 aa, Artificial Sequence
  Chemically synthesized peptide
  misc_feature (1)..(1): Xaa is none or a natural, unnatural or an analogue amino acid, may be L, D, or a methylated amino acid, or any combination
  misc_feature (2)..(2): Xaa is none or a natural, unnatural or an analogue amino acid, may be L, D, or a methylated amino acid, or any combination
  misc_feature (3)..(3): Xaa is any natural, unnatural or an analogue amino acid, may be L, D, or a methylated amino acid, or any combination
  misc_feature (5)..(6): Xaa is a natural, unnatural, analogue, L, D, or methylated amino acid or any combination
  misc_feature (8)..(11): Xaa is a natural, unnatural, analogue, L, D, or methylated amino acid or any combination
  misc_feature (13)..(14): Xaa is a natural, unnatural, analogue, L, D, or methylated amino acid or any combination
  misc_feature (16)..(16): Xaa is none or one natural, unnatural or an analogue amino acid, may be L, D, or a methylated amino acid, or any combination
XXXCXXCXXXXCXXCX

SEQ ID NO: 297 - protein, 16 aa, Artificial Sequence
  Chemically synthesized peptide
  misc_feature (1)..(1): Xaa is none or a natural, unnatural or an analogue amino acid, may be L, D, or a methylated amino acid, or any combination
  misc_feature (2)..(2): Xaa is none or a natural, unnatural or an analogue amino acid, may be L, D, or a methylated amino acid, or any combination
  misc_feature (3)..(3): Xaa is any natural, unnatural or an analogue amino acid, may be L, D, or a methylated amino acid, or any combination
  misc_feature (4)..(4): Xaa is a cysteine, penicillamine homocysteine, or 3-mercaptoproline
  misc_feature (6)..(6): Xaa is a natural, unnatural, analogue, L, D, or methylated amino acid or any combination
  misc_feature (7)..(7): Xaa is a cysteine, penicillamine homocysteine, or 3-mercaptoproline
  misc_feature (12)..(12): Xaa is a cysteine, penicillamine homocysteine, or 3-mercaptoproline
  misc_feature (15)..(15): Xaa is a cysteine, penicillamine homocysteine, or 3-mercaptoproline
  misc_feature (16)..(16): Xaa is none or one natural, unnatural or an analogue amino acid, may be L, D, or a methylated amino acid, or any combination
XXXXEXXVNVAXTGXX

SEQ ID NO: 298 - protein, 16 aa, Artificial Sequence
  Chemically synthesized peptide
  misc_feature (1)..(1): Xaa is none or a natural, unnatural or an analogue amino acid, may be L, D, or a methylated amino acid, or any combination
  misc_feature (2)..(2): Xaa is none or a natural, unnatural or an analogue amino acid, may be L, D, or a methylated amino acid, or any combination
  misc_feature (3)..(3): Xaa is any natural, unnatural or an analogue amino acid, may be L, D, or a methylated amino acid, or any combination
  misc_feature (4)..(4): Xaa is a cysteine, penicillamine homocysteine, or 3-mercaptoproline
  misc_feature (5)..(6): Xaa is a natural, unnatural, analogue, L, D, or methylated amino acid or any combination
  misc_feature (7)..(7): Xaa is a cysteine, penicillamine homocysteine, or 3-mercaptoproline
  misc_feature (8)..(11): Xaa is a natural, unnatural, analogue, L, D, or methylated amino acid or any combination
  misc_feature (12)..(12): Xaa is a cysteine, penicillamine homocysteine, or 3-mercaptoproline
  misc_feature (13)..(14): Xaa is a natural, unnatural, analogue, L, D, or methylated amino acid or any combination
  misc_feature (15)..(15): Xaa is a cysteine, penicillamine homocysteine, or 3-mercaptoproline
  misc_feature (16)..(16): Xaa is none or one natural, unnatural or an analogue amino acid, may be L, D, or a methylated amino acid, or any combination
XXXXXXXXXXXXXXXX

SEQ ID NO: 299 - protein, 16 aa, Artificial Sequence
  Chemically synthesized peptide
  misc_feature (3)..(3): Xaa is alpha-aminoadipic acid
  misc_feature (5)..(6): Xaa is a natural, unnatural, analogue, L, D, or methylated amino acid or any combination
  misc_feature (8)..(8): Xaa is a natural, unnatural, analogue, L, D, or methylated amino acid or any combination
  misc_feature (10)..(11): Xaa is a natural, unnatural, analogue, L, D, or methylated amino acid or any combination
  misc_feature (13)..(14): Xaa is a natural, unnatural, analogue, L, D, or methylated amino acid or any combination
  misc_feature (16)..(16): Xaa is a natural, unnatural, analogue, L, D, or methylated amino acid or any combination
NDXCXXCXNXXCXXCX

SEQ ID NO: 300 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (5)..(6): Xaa is a natural, unnatural, analogue, L, D, or methylated amino acid or any combination
  misc_feature (8)..(8): Xaa is a natural, unnatural, analogue, L, D, or methylated amino acid or any combination
  misc_feature (10)..(11): Xaa is a natural, unnatural, analogue, L, D, or methylated amino acid or any combination
  misc_feature (13)..(14): Xaa is a natural, unnatural, analogue, L, D, or methylated amino acid or any combination
  misc_feature (16)..(16): Xaa is a natural, unnatural, analogue, L, D, or methylated amino acid or any combination
NEXCXXCXNXXCXXCX

SEQ ID NO: 301 - protein, 16 aa, Artificial Sequence
  Chemically synthesized peptide
  misc_feature (1)..(1): Asn is a D amino acid
  misc_feature (2)..(2): Glu is a D amino acid
  misc_feature (3)..(3): Xaa is alpha-aminoadipic acid
  misc_feature (5)..(6): Xaa is a natural, unnatural, analogue, L, D, or methylated amino acid or any combination
  misc_feature (8)..(8): Xaa is a natural, unnatural, analogue, L, D, or methylated amino acid or any combination
  misc_feature (10)..(11): Xaa is a natural, unnatural, analogue, L, D, or methylated amino acid or any combination
  misc_feature (13)..(14): Xaa is a natural, unnatural, analogue, L, D, or methylated amino acid or any combination
  misc_feature (16)..(16): Xaa is a natural, unnatural, analogue, L, D, or methylated amino acid or any combination
NEXCXXCXNXXCXXCX

SEQ ID NO: 302 - protein, 16 aa, Artificial Sequence
  Chemically synthesized peptide
  misc_feature (1)..(1): Asn is a D amino acid
  misc_feature (2)..(2): Asp is a D amino acid
  misc_feature (3)..(3): Xaa is alpha-aminoadipic acid
  misc_feature (5)..(6): Xaa is a natural, unnatural, analogue, L, D, or methylated amino acid or any combination
  misc_feature (8)..(8): Xaa is a natural, unnatural, analogue, L, D, or methylated amino acid or any combination
  misc_feature (10)..(11): Xaa is a natural, unnatural, analogue, L, D, or methylated amino acid or any combination
  misc_feature (13)..(14): Xaa is a natural, unnatural, analogue, L, D, or methylated amino acid or any combination
  misc_feature (16)..(16): Xaa is a natural, unnatural, analogue, L, D, or methylated amino acid or any combination
NDXCXXCXNXXCXXCX

SEQ ID NO: 303 - protein, 16 aa, Artificial Sequence
  Chemically synthesized peptide
  misc_feature (1)..(1): Asn is a D amino acid
  misc_feature (2)..(2): Asp is a D amino acid
  misc_feature (3)..(3): Xaa is alpha-aminoadipic acid
  misc_feature (5)..(6): Xaa is a natural, unnatural, analogue, L, D, or methylated amino acid or any combination
  misc_feature (8)..(8): Xaa is a natural, unnatural, analogue, L, D, or methylated amino acid or any combination
  misc_feature (10)..(11): Xaa is a natural, unnatural, analogue, L, D, or methylated amino acid or any combination
  misc_feature (13)..(14): Xaa is a natural, unnatural, analogue, L, D, or methylated amino acid or any combination
  misc_feature (16)..(16): Xaa is a natural, unnatural, analogue, L, D, or methylated amino acid or any combination
NDXCXXCXYXXCXXCX

SEQ ID NO: 304 - protein, 16 aa, Artificial Sequence
  Chemically synthesized peptide
  misc_feature (1)..(1): Asn is a D amino acid
  misc_feature (2)..(2): Glu is a D amino acid
  misc_feature (3)..(3): Xaa is alpha-aminoadipic acid
  misc_feature (5)..(6): Xaa is a natural, unnatural, analogue, L, D, or methylated amino acid or any combination
  misc_feature (8)..(8): Xaa is a natural, unnatural, analogue, L, D, or methylated amino acid or any combination
  misc_feature (10)..(11): Xaa is a natural, unnatural, analogue, L, D, or methylated amino acid or any combination
  misc_feature (13)..(14): Xaa is a natural, unnatural, analogue, L, D, or methylated amino acid or any combination
  misc_feature (16)..(16): Xaa is a natural, unnatural, analogue, L, D, or methylated amino acid or any combination
NEXCXXCXYXXCXXCX

SEQ ID NO: 305 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Leu is a D amino acid
  misc_feature (16)..(16): Leu is conjugated to an amide
NDXCELCVNVACTGCL

SEQ ID NO: 306 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Ser is a D amino acid
  misc_feature (16)..(16): Ser is conjugated to an amide
NDXCELCVNVACTGCS

SEQ ID NO: 307 - protein, 16 aa, Artificial Sequence
  Chemically synthesized peptide
  misc_feature (1)..(1): Asn is a D amino acid
  misc_feature (3)..(3): Xaa is alpha-aminoadipic acid
  misc_feature (16)..(16): Tyr is a D amino acid
  misc_feature (16)..(16): Tyr is conjugated to an amide
NDXCELCVNVACTGCY

SEQ ID NO: 308 - protein, 16 aa, Artificial Sequence
  Chemically synthesized peptide
  misc_feature (1)..(1): Glu is conjugated to Py
  misc_feature (3)..(3): Xaa is alpha-aminoadipic acid
EDXCELCVNVACTGCL

SEQ ID NO: 309 - protein, 16 aa, Artificial Sequence
  Chemically synthesized peptide
  misc_feature (1)..(1): Asn is attached to polyethylene glycol
  misc_feature (3)..(3): Xaa is alpha-aminoadipic acid
  misc_feature (16)..(16): Leu is attached to polyethylene glycol
NDXCELCVNVACTGCL

SEQ ID NO: 310 - protein, 16 aa, Artificial Sequence
  Chemically synthesized peptide
  misc_feature (1)..(1): Asn is attached to polyethylene glycol
  misc_feature (3)..(3): Xaa is alpha-aminoadipic acid
NDXCELCVNVACTGCL

SEQ ID NO: 311 - protein, 16 aa, Artificial Sequence
  Chemically synthesized peptide
  misc_feature (5)..(5): Xaa is alpha-aminoadipic acid
  misc_feature (16)..(16): Leu is attached to polyethylene glycol
NDECXLCVNVACTGCL

SEQ ID NO: 312 - protein, 16 aa, Artificial Sequence
  Chemically synthesized peptide
  misc_feature (1)..(2): Xaa is a natural, unnatural, analogue, L, D, or methylated amino acid or any combination
  misc_feature (3)..(3): Xaa is alpha-aminoadipic acid
  misc_feature (4)..(4): Xaa is a cysteine, penicillamine homocysteine, or 3-mercaptoproline
  misc_feature (5)..(6): Xaa is a natural, unnatural, analogue, L, D, or methylated amino acid or any combination
  misc_feature (7)..(7): Xaa is a cysteine, penicillamine homocysteine, or 3-mercaptoproline
  misc_feature (8)..(11): Xaa is a natural, unnatural, analogue, L, D, or methylated amino acid or any combination
  misc_feature (12)..(12): Xaa is a cysteine, penicillamine homocysteine, or 3-mercaptoproline
  misc_feature (13)..(14): Xaa is a natural, unnatural, analogue, L, D, or methylated amino acid or any combination
  misc_feature (15)..(15): Xaa is a cysteine, penicillamine homocysteine, or 3-mercaptoproline
  misc_feature (16)..(16): Xaa is a natural, unnatural, analogue, L, D, or methylated amino acid or any combination
XXXXXXXXXXXXXXXX

SEQ ID NO: 313 - protein, 16 aa, Artificial Sequence
  Chemically synthesized peptide
  misc_feature (3)..(3): Xaa is alpha-aminoadipic acid
EDXCELCVNVACTGCL

SEQ ID NO: 314 - protein, 16 aa, Artificial Sequence
  Chemically synthesized peptide
  misc_feature (3)..(3): Xaa is alpha-aminoadipic acid
EEXCELCVNVACTGCL

SEQ ID NO: 315 - protein, 16 aa, Artificial Sequence
  Chemically synthesized peptide
  misc_feature (3)..(3): Xaa is alpha-aminoadipic acid
DDXCELCVNVACTGCL

SEQ ID NO: 316 - protein, 16 aa, Artificial Sequence
  Chemically synthesized peptide
  misc_feature (3)..(3): Xaa is alpha-aminoadipic acid
DEXCELCVNVACTGCL

SEQ ID NO: 317 - protein, 16 aa, Artificial Sequence
  Chemically synthesized peptide
  misc_feature (3)..(3): Xaa is alpha-aminoadipic acid
QDXCELCVNVACTGCL

SEQ ID NO: 318 - protein, 16 aa, Artificial Sequence
  Chemically synthesized peptide
  misc_feature (3)..(3): Xaa is alpha-aminoadipic acid
QEXCELCVNVACTGCL

SEQ ID NO: 319 - protein, 16 aa, Artificial Sequence
  Chemically synthesized peptide
  misc_feature (3)..(3): Xaa is alpha-aminoadipic acid
KDXCELCVNVACTGCL

SEQ ID NO: 320 - protein, 16 aa, Artificial Sequence
  Chemically synthesized peptide
  misc_feature (3)..(3): Xaa is alpha-aminoadipic acid
KEXCELCVNVACTGCL

SEQ ID NO: 321 - protein, 16 aa, Artificial Sequence
  Chemically synthesized peptide
  misc_feature (3)..(3): Xaa is alpha-aminoadipic acid
EDXCELCVNVACTGCS

SEQ ID NO: 322 - protein, 16 aa, Artificial Sequence
  Chemically synthesized peptide
  misc_feature (3)..(3): Xaa is alpha-aminoadipic acid
EEXCELCVNVACTGCS

SEQ ID NO: 323 - protein, 16 aa, Artificial Sequence
  Chemically synthesized peptide
  misc_feature (3)..(3): Xaa is alpha-aminoadipic acid
DDXCELCVNVACTGCS

SEQ ID NO: 324 - protein, 16 aa, Artificial Sequence
  Chemically synthesized peptide
  misc_feature (3)..(3): Xaa is alpha-aminoadipic acid
DEXCELCVNVACTGCS

SEQ ID NO: 325 - protein, 16 aa, Artificial Sequence
  Chemically synthesized peptide
  misc_feature (3)..(3): Xaa is alpha-aminoadipic acid
QDXCELCVNVACTGCS

SEQ ID NO: 326 - protein, 16 aa, Artificial Sequence
  Chemically synthesized peptide
  misc_feature (3)..(3): Xaa is alpha-aminoadipic acid
QEXCELCVNVACTGCS

SEQ ID NO: 327 - protein, 16 aa, Artificial Sequence
  Chemically synthesized peptide
  misc_feature (3)..(3): Xaa is alpha-aminoadipic acid
KDXCELCVNVACTGCS

SEQ ID NO: 328 - protein, 16 aa, Artificial Sequence
  Chemically synthesized peptide
  misc_feature (3)..(3): Xaa is alpha-aminoadipic acid
KEXCELCVNVACTGCS

SEQ ID NO: 329 - protein, 16 aa, Artificial Sequence
  Chemically synthesized peptide
  misc_feature (3)..(3): Xaa is alpha-aminoadipic acid
EDXCELCINMACTGCL

SEQ ID NO: 330 - protein, 16 aa, Artificial Sequence
  Chemically synthesized peptide
  misc_feature (3)..(3): Xaa is alpha-aminoadipic acid
EEXCELCINMACTGCL

SEQ ID NO: 331 - protein, 16 aa, Artificial Sequence
  Chemically synthesized peptide
  misc_feature (3)..(3): Xaa is alpha-aminoadipic acid
DDXCELCINMACTGCL

SEQ ID NO: 332 - protein, 16 aa, Artificial Sequence
  Chemically synthesized peptide
  misc_feature (3)..(3): Xaa is alpha-aminoadipic acid
DEXCELCINMACTGCL

SEQ ID NO: 333 - protein, 16 aa, Artificial Sequence
  Chemically synthesized peptide
  misc_feature (3)..(3): Xaa is alpha-aminoadipic acid
QDXCELCINMACTGCL

SEQ ID NO: 334 - protein, 16 aa, Artificial Sequence
  Chemically synthesized peptide
  misc_feature (3)..(3): Xaa is alpha-aminoadipic acid
QEXCELCINMACTGCL

SEQ ID NO: 335 - protein, 16 aa, Artificial Sequence
  Chemically synthesized peptide
  misc_feature (3)..(3): Xaa is alpha-aminoadipic acid
KDXCELCINMACTGCL

SEQ ID NO: 336 - protein, 16 aa, Artificial Sequence
  Chemically synthesized peptide
  misc_feature (3)..(3): Xaa is alpha-aminoadipic acid
KEXCELCINMACTGCL

SEQ ID NO: 337 - protein, 16 aa, Artificial Sequence
  Chemically synthesized peptide
  misc_feature (3)..(3): Xaa is alpha-aminoadipic acid
EDXCELCINMACTGCS

SEQ ID NO: 338 - protein, 16 aa, Artificial Sequence
  Chemically synthesized peptide
  misc_feature (3)..(3): Xaa is alpha-aminoadipic acid
EEXCELCINMACTGCS

SEQ ID NO: 339 - protein, 16 aa, Artificial Sequence
  Chemically synthesized peptide
  misc_feature (3)..(3): Xaa is alpha-aminoadipic acid
DDXCELCINMACTGCS

SEQ ID NO: 340 - protein, 16 aa, Artificial Sequence
  Chemically synthesized peptide
  misc_feature (3)..(3): Xaa is alpha-aminoadipic acid
DEXCELCINMACTGCS

SEQ ID NO: 341 - protein, 16 aa, Artificial Sequence
  Chemically synthesized peptide
  misc_feature (3)..(3): Xaa is alpha-aminoadipic acid
DEXCELCINMACTGCS

SEQ ID NO: 342 - protein, 16 aa, Artificial Sequence
  Chemically synthesized peptide
  misc_feature (3)..(3): Xaa is alpha-aminoadipic acid
QDXCELCINMACTGCS

SEQ ID NO: 343 - protein, 16 aa, Artificial Sequence
  Chemically synthesized peptide
QEDCELCINMACTGCS

SEQ ID NO: 344 - protein, 16 aa, Artificial Sequence
  Chemically synthesized peptide
  misc_feature (3)..(3): Xaa is alpha-aminoadipic acid
QEXCELCINMACTGCS

SEQ ID NO: 345 - protein, 16 aa, Artificial Sequence
  Chemically synthesized peptide
  misc_feature (3)..(3): Xaa is alpha-aminoadipic acid
KDXCELCINMACTGCS

SEQ ID NO: 346 - protein, 16 aa, Artificial Sequence
  Chemically synthesized peptide
  misc_feature (3)..(3): Xaa is alpha-aminoadipic acid
KEXCELCINMACTGCS

SEQ ID NO: 347 - protein, 16 aa, Artificial Sequence
  Chemically synthesized peptide
  misc_feature (3)..(3): Xaa is alpha-aminoadipic acid
NDXCELCVNVACTGCT

SEQ ID NO: 348 - protein, 16 aa, Artificial Sequence
  Chemically synthesized peptide
  misc_feature (3)..(3): Xaa is alpha-aminoadipic acid
NDXCELCVNVACTGCY

(19) United States
(12) Patent Application Publication
(10) Pub. No.: US 2020/0023080 A1
(43) Pub. Date: Jan. 23, 2020

(54) COMPOSITIONS AND METHOD FOR THE TREATMENT AND DETECTION OF COLON CANCER

(71) Applicant: Bausch Health Ireland Limited, Dublin (IE)

(72) Inventors: Kunwar SHAILUBHAI, Audubon, PA (US); Vaseem PALEJWALA, South Plains, NJ (US)

(21) Appl. No.: 15/777,273

(22) PCT Filed: Nov. 18, 2016
(86) PCT No.: PCT/US2016/062902

§ 371 Date: May 18, 2018

Related U.S. Application Data
(60) Provisional application No. 62/256,927, filed on Nov. 18, 2015.

(51) Int. Cl. A61K 49/00; A61K 47/62; A61K 47/69; A61K 51/12; A61K 51/08; A61P 35/00

(52) U.S. Cl. CPC A61K 49/0056; A61K 47/62; A61K 47/6937; A61K 49/0032; A61K 51/088; A61P 35/00; A61K 51/1244

(57) ABSTRACT

The present invention provides methods of detecting and treating colon cancer using conjugates comprising a GCRA peptide and one or more components such as a drug delivery vehicle, a chemotherapeutic agent, a micelle, a nanoparticle, a liposome, a polymer, a lipid, an imaging agent, and a labeling agent.

### GOVERNMENT INTEREST

This invention was made with government support under CA165207-01 awarded by the National Cancer Institute. The government has certain rights in the invention.

### FIELD OF THE INVENTION

The present invention relates generally to compositions and methods for the early detection and treatment of colon cancer and its metastases.

### BACKGROUND OF THE INVENTION

Cancer of the colon is a common and deadly disease in the Western world. Genetic predisposition plays an important role, but exposure to substances that initiate and promote cancer is essential for a malignant tumor to develop.

In the normal colon mucosa, epithelial cells lining the upper ⅔ of the crypt along the mucosal wall are normally non-proliferating, while those lining the lower ⅓ of the crypts are proliferating. As the proliferating cells migrate toward the upper portion of the crypt they transform and lose their proliferative ability. Ultimately the oldest cells are shed from the colon surface in the normal functioning of the colon. However, when the proliferating epithelial cells are induced to retain their proliferative capacity after reaching the upper ⅓ of the crypt, the normal process may go awry and microadenomas form. The proliferating cell, now at the surface of the colon continues to proliferate and a polyp develops.

Polyps may be either benign or cancerous. Some never become cancerous, but it is believed that adenomatous polyps are the main precursors of colon cancer and that about 90% of colon cancers develop from adenomatous polyps. Most adenomas do not continue to grow in size, but those that do are more likely to develop malignant changes. Particularly, flat colorectal dysplasias are often missed during endoscopy for colon cancer screening, but it has been demonstrate that the flat lesions within the lining of the colon and rectum are more likely to be cancerous than polyps. Thus, there is an unmet need to develop diagnostic methods for specific detection of Dysplasias, including polypoid indeterminate, and the flat dysplastic lesions in colon and rectum. Therefore, reducing the number of adenomas and/or preventing their growth substantially reduces the number of potential colon cancers in the future.

There is therefore a need for a method for the early detection, treatment and prevention of colorectal cancer.

### SUMMARY OF THE INVENTION

In various aspects the invention provides a conjugate of a GCRA peptide and at least one component, where the component is optionally attached by a spacer. The component is a drug delivery vehicle, a chemotherapeutic agent, a micelle, a nanoparticle, a liposome, a polymer, a lipid, an imaging agent, or a labeling agent. The GCRA peptide is a peptide having the amino acid sequence of any one of Tables 1-8. Preferably, the GCRA peptide is SEQ ID NO: 1 (SP-304), SP332 (SEQ ID NO: 8), SEQ ID NO.:9 (SP-333), SP364 (SEQ ID NO.:100), or SP366 (SEQ ID NO.:102). The peptide and component is covalently coupled directly or via a linker. Preferably the linker is 4 to 10 carbons in length. The component is conjugated to the N-terminus of the GCRA peptide.

The drug delivery vehicle is a liposome, a polymeric micelle, a lipoprotein-based drug carrier, a nanoparticle drug carrier, or dendrimers. Preferably the nanoparticle is a PLA- or PLGA-based nanoparticle. The drug delivery vehicle further includes at least one chemotherapeutic agent encapsulated within the drug delivery vehicle. The chemotherapeutic agent is for example, a taxane, an anthracyclin, a platin or 5-fluorouracil or any combination thereof.

The imaging agent is a florescence dye or radioactive moiety. The labeling agent could also be biotin, followed by detection with avidin or streptavidin conjugated to fluorescence dye.

The GCRA peptide may be a peptide that does not contain a D-amino acid at the N-terminus. The GCRA peptide may be a peptide that contains a D-amino acid at the C-terminus. The GCRA peptide may be a peptide that does not contain a D-amino acid at the N-terminus and contains a D-amino acid at the C-terminus.

The GCRA peptide may be conjugated to two components. The GCRA peptide may conjugated to a nanoparticle and an imaging agent. The GCRA peptide may be conjugated to a nanoparticle and a chemotherapeutic. The nanoparticle may be conjugated to the C-terminus of the GCRA peptide. The imaging agent may be conjugated to the N-terminus of the GCRA peptide and the nanoparticle may be conjugated to the C-terminus of the GCRA peptide. The chemotherapeutic agent may be conjugated to the N-terminus of the GCRA peptide and the nanoparticle may be conjugated to the C-terminus of the GCRA peptide.

Also included in the invention is a composition comprising the conjugate according to the invention and a pharmaceutically acceptable excipient.

In other aspects the invention provides methods for the reduction of the incidence of colonic adenomas and colonic microadenomas in subject by administering to the subject a composition according to the invention, wherein the component is chemotherapeutic agent or a drug delivery vehicle, a micelle, a nanoparticle, a liposome, a polymer, a lipid having at least one chemotherapeutic agent encapsulated within.

In yet a further aspect the invention provides methods for reducing the incidence of colon cancer in subject by administering to the subject a composition according to the invention, wherein the component is chemotherapeutic agent or a drug delivery vehicle, a micelle, a nanoparticle, a liposome, a polymer, a lipid having at least one chemotherapeutic agent encapsulated within.

In other aspects the invention provides methods for reducing the incidence of colon cancer metastasis in subject comprising administering to the subject a composition according to the invention, wherein the component is chemotherapeutic agent or a drug delivery vehicle, a micelle, a nanoparticle, a liposome, a polymer, a lipid having at least one chemotherapeutic agent encapsulated within.

In yet further aspects the invention provides methods of lowering the risk of adenoma development in subject by administering to the subject a composition according to the invention, wherein the component is chemotherapeutic agent or a drug delivery vehicle, a micelle, a nanoparticle, a liposome, a polymer, a lipid having at least one chemotherapeutic agent encapsulated within.

In another aspect the invention provides methods f treating colon cancer metastases in subject by administering to the subject a composition according to the invention, wherein the component is chemotherapeutic agent or a drug delivery vehicle, a micelle, a nanoparticle, a liposome, a polymer, a lipid having at least one chemotherapeutic agent encapsulated within.

The subject is at risk of developing such adenomas or microadenomas. The subject has been diagnosed with colon cancer, colonic adenomas, colonic microadenomas or colonic polyps or has a close blood relative diagnosed with colon cancer, colonic adenomas, colonic microadenomas or colonic polyps.

In still another aspect the invention provides methods of detecting a colonic microadenoma or a flat colorectal dysplasias by contacting colonic tissue with a composition according to the invention, wherein the component is an imaging agent or labeling agent.

In other aspects the invention provides methods of detecting colorectal cancer metastasis or flat colorectal dysplasias by administering to the subject a composition according to the invention, wherein the component is an imaging agent and detecting said imaging agent in the subject.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1: is a schematic illustrating the synthesis screen for APi578 IIRDye680LT-SP-333)

FIG. 2 A-B show exemplary therapeutics of the present disclosure. Panel A shows a GCRA peptide linked by a 10-carbon spacer arm to a chemotherapeutic drug. The C-terminus of the GCRA peptide can be any D-amino acid, and the length of the spacer arm can be between 4 and 10 carbons long, with a length of 10-carbons being preferred. Panel B shows GCRA peptide-drug conjugates loaded on nanoparticles.

FIG. 3 shows the dose curve for the binding assay with IR680LT SP333

FIG. 4 shows the binding assay with IR680LT SP333 at different cell densities

FIG. 5 illustrates the specify of IR680LT SP333 for GC-C receptors.

FIG. 6 shows the potency of unconjugated SP-333 (SEQ ID NO: 9) and SP-333 conjugated to IR680 or biotin in T84 cGMP bioassay. IR680 NHS ester (-5-6 carbon equivalent) is directly conjugated to SP-333 while biotin is conjugated to SP-333 via 2 amino ethoxy acetic acid (AEEA; each ˜9-10 carbon chain equivalent). Both conjugates are active as evidenced by their ability to stimulate cGMP production in T84 cells although at a slightly lower level as compared to unconjugated SP-333.

FIG. 7A-B shows ex-vivo analysis of the SP-333-IR680 probe in Apc^{+/Min-FCCC} mice. Colons were excised from mice 2 months (panel A) and 3 months (panel B) of age, incubated with 0.1 μM SP-333-IR680 probe for 30 min ex vivo and imaged in the IVIS Spectrum system. Each panel contains a white light image (left) and corresponding fluorescent image (right) of the same full-length colon. Circles denote the location of colon tumors. Blue arrows indicate the corresponding images of the same tumor.

FIG. 8A-B shows loss of SP-333-IR680 binding affinity in colonic tumors from older Apc^{+/Min-FCCC} mice. Colons were excised from 4 month (panel A) and 6 month (panel B) old Apc^{+/Min-FCCC} mice and incubated with 0.1 μM SP-333-IR680 probe for 30 min ex vivo. Colon tumors are circled. Blue arrows indicate the corresponding white light (left) and fluorescent (right) images of gross tumors. The signal is weaker than that observed in younger animals.

FIG. 9A-C shows images from Apc^{+/Min-FCCC} mice gavaged with different doses of IR680-SP-333 and imaged at post treatment interval times. A) image of mouse gavaged with IR680-SP-333 probe at 0.05 mg/kg body weight. B) increasing the probe dose to 0.5 mg/kg body weight failed to improve the difference in signal between neoplastic and nonneoplastic mucosa significantly. However, the nonspecific signal at both proximal and distal ends of colon increased significantly. C) image acquired 90 min after administration of IR680-SP-333 at 0.5 mg/kg body weight.

FIG. 10 is an image showing a mouse colon gavaged with IR680LT SP333. The strongest probe signal of the colon of mouse 12281 was observed in areas of hyperproliferation (HP) followed by an adenoma with intermediate signal intensity. A slight increase in signal was observed in a micro-adenoma as compared to the surrounding normal colonic mucosa. FIG. 11 is an image showing a mouse colon gavaged with IR680LT SP333. The colon of mouse ID 12276 exhibits a very strong probe signal in small gross lesion (1-mm in size) and a weaker signal in a flat/indeterminate adenoma.

FIG. 12 is an image showing a mouse colon gavaged with IR680LT SP333. A strong signal is localized to a hyperplastic polyp (HP); an area of high proliferation; providing additional support for the lack of correlation between the intensity of the probe signal and size/type of colonic lesion.

### DETAILED DESCRIPTION OF THE INVENTION

Guanylate cyclase C (GC-C) is a type I transmembrane glycoprotein expressed on brush border membranes of intestinal epithelial cells, as well as on transformed human colon carcinoma cells such as the T-84 cell line. GC-C is a receptor selectively expressed by histologically confirmed human primary colorectal tumors and metastases, while normal tissues and other types of cancer either lack or express very low levels of GC-C receptors. Its persistent expression by colorectal carcinomas and ectopic expression by adenocarcinomas of the upper gastrointestinal (GI) tract suggest its use as a biomarker for GI malignancies and provide strong rationale for the development of GC-C agonist-based in vivo imaging probes for the detection of colorectal cancer. Uroguanylin, a physiological ligand of GC-C, is a 16 amino acid peptide with two disulfide bonds and nanomolar affinity for the GC-C receptor. Plecanatide (formerly known as SP-304) and dolcanatide (formerly known as SP-333) are more stable analogs of uroguanylin. These peptides bind specifically to GC-C, and therefore could be used for specific detection of colorectal polyps, tumors, various types of dysplastic lesions and the colorectal metastases.

The invention is based in part upon the discovery that a guanylate cyclase C receptor agonist peptide conjugated to a florescent dye was capable of detecting colon tumors. Importantly, the probe did not detect all colon tumors but ones that are new and still growing. Accordingly, guanylate cyclase C receptor agonist peptides conjugated to detectable labels are useful in detecting early stage colon cancer, in particular colonic adenomas, microadenomas and metastases.

Accordingly the invention provides a conjugate containing a GCRA peptide; and one or more components, to which the GCRA peptide is conjugated. The component is a drug delivery vehicle, a chemotherapeutic agent, a micelle, a nanoparticle, a liposome, a polymer, a lipid, an imaging agent, and a labeling agent. The peptide and component is covalently coupled directly or via a linker. The conjugates are useful in methods of reduction of the incidence of colonic adenomas and colonic microadenomas, reducing the incidence of colon cancer, reducing the incidence of or treating colon cancer metastasis in subject. The conjugates are also useful in screening methods to detect colonic microadenomas or colorectal cancer metastasis

GCRA Peptides

The GCRA peptides of the present invention are analogues of, uroguanylin, guanylin, lymphoguanylin and ST peptides. No particular length is implied by the term “peptide”. In some embodiments, the GCRA peptide is less than 25 amino acids in length, e.g., less than or equal to 20, 15, 14, 13, 12, 11, 10, or 5 amino acid in length.

The GCRA peptides can be polymers of L-amino acids, D-amino acids, or a combination of both. For example, in various embodiments, the peptides are D retro-inverso peptides. The term “retro-inverso isomer” refers to an isomer of a linear peptide in which the direction of the sequence is reversed and the chirality of each amino acid residue is inverted. See, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into a D retro-inverso peptide by synthesizing a reverse of the sequence for the corresponding native L-amino acid sequence. For example a GCRA peptide includes the sequence defined by Formulas I-XX and those listed on Tables 2-8.

By inducing cGMP production is meant that the GCRA peptide induces the production of intracellular cGMP. Intracellular cGMP is measured by methods known in the art. For example, the GCRA peptide of the invention stimulate 5%, 10%, 20%, 30%, 40%, 50% , 75%, 90% or more intracellular cGMP compared to naturally occurring GC-C agonists. In further embodiments, the GCRA peptide stimulates apoptosis, e.g., programmed cell death or activates the cystic fibrosis transmembrane conductance regulator (CFTR).

As used herein PEG3, 3 PEG, is meant to denote polyethylene glycol such as include aminoethyloxy-ethyloxy-acetic acid (AeeA).

As used herein, the term “AMIDE” is meant to denote that the terminal carboxylic acid is replaced with an amide group, i.e., the terminal COOH is replaced with CONH₂.

As used herein, the term “pyGlu” refers to pyroglutamic acid.

As used herein, (e.g., in Formulas I-XX) Xₐₐ is any natural, unnatural amino acid or amino acid analogue; Mₐₐ is a Cysteine (Cys), Penicillamine (Pen) homocysteine, or 3-mercaptoproline. Xaaₙ₁ is meant to denote an amino acid sequence of any natural, unnatural amino acid or amino acid analogue that is one, two or three residues in length; Xaaₙ₂ is meant to denote an amino acid sequence of any natural, unnatural amino acid or amino acid analogue that is zero or one residue in length; and Xaaₙ₃ is meant to denote an amino acid sequence of any natural, unnatural amino acid or amino acid analogue that is zero, one, two, three, four, five or six residues in length. Additionally, any amino acid represented by Xaa, may be an L-amino acid, a D-amino acid, a methylated amino acid, a fluorinated amino acid or any combination of thereof. Preferably the amino acids at the N-terminus, C-terminus or both are D-amino acids. Optionally, any GCRA peptide represented by Formulas I-XX may contain on or more polyethylene glycol residues at the N-terminus, C-terminus or both. An exemplary polyethylene glycol includes aminoethyloxy-ethyloxy-acetic acid and polymers thereof.

Specific examples of GCC agonist peptides that can be used in the methods and formulations of the invention include a peptide selected from Tables 2-8. In some embodiments, the GCC agonist peptide is SEQ ID NO: 1 (SP-304). In some embodiments, the GCC agonist peptide is SEQ ID NO: 9 (SP-333). In some embodiments, the GCC agonist peptide is SEQ ID NO: 8 (SP-332). In some embodiments, the GCC agonist peptide contains a D-amino acid at the N-terminus. In some embodiments, the GCC agonist peptide contains a D-amino acid at the C-terminus. In some embodiments, the GCC agonist peptide contains a D-amino acid at both the N- and C-termini. In some embodiments, the GCC agonist peptide does not contain a D-amino acid at both the N- and C-termini. In some embodiments, the GCC agonist peptide does not contain a D-amino acid at the C-terminus and contains a D-amino acid at the N-terminus. In some embodiments, the GCC agonist peptide does not contain a D-amino acid at the N-terminus and contains a D-amino acid at the C-terminus.

In some embodiments, GCC agonist peptides include peptides having the amino acid sequence of Formula I, wherein at least one amino acid of Formula I is a D-amino acid or a methylated amino acid and/or the amino acid at position 16 is a serine. Preferably, the amino acid at position 16 of Formula I is a D-amino acid or a methylated amino acid. For example, the amino acid at position 16 of Formula I is a d-leucine or a d-serine. Optionally, one or more of the amino acids at positions 1-3 of Formula I are D-amino acids or methylated amino acids or a combination of D-amino acids or methylated amino acids. For example, Asn¹, Asp² or Glu³ (or a combination thereof) of Formula I is a D-amino acid or a methylated amino acid. Preferably, the amino acid at position Xaa⁶ of Formula I is a leucine, serine or tyrosine.

In alternative embodiments, GCC agonist peptides include peptides having the amino acid sequence of Formula II, wherein at least one amino acid of Formula II is a D-amino acid or a methylated amino acid. Preferably, the amino acid denoted by Xaaₙ₂ of Formula II is a D-amino acid or a methylated amino acid. In some embodiments, the amino acid denoted by Xaaₙ₂ of Formula II is a leucine, a d-leucine, a serine, or a d-serine. Preferably, the one or more amino acids denoted by Xaaₙ₁ of Formula II are D-amino acids or methylated amino acids. Preferably, the amino acid at position Xaa⁶ of Formula II is a leucine, a serine, or a tyrosine.

In some embodiments, GCC agonist peptides include peptides having the amino acid sequence of Formula III, wherein at least one amino acid of Formula III is a D-amino acid or a methylated amino acid and/or Maa is not a cysteine. Preferably, the amino acid denoted by Xaaₙ₂ of Formula III is a D-amino acid or a methylated amino acid. In some embodiments the amino acid denoted by Xaaₙ₂ of Formula III is a leucine, a d-leucine, a serine, or a d-serine. Preferably, the one or more amino acids denoted by Xaaₙ₁ of Formula III are D-amino acids or methylated amino acids. Preferably, the amino acid at position Xaa⁶ of Formula III is a leucine, a serine, or a tyrosine.

In other embodiments, GCC agonist peptides include peptides having the amino acid sequence of Formula IV, wherein at least one amino acid of Formula IV is a D-amino acid or a methylated amino acid, and/or Maa is not a cysteine. Preferably, the Xaaₙ₂ of Formula IV is a D-amino acid or a methylated amino acid. In some embodiments, the amino acid denoted by Xaaₙ₂ of Formula IV is a leucine, a d-leucine, a serine, or a d-serine. Preferably, the one or more of the amino acids denoted by Xaaₙ₁ of Formula IV are D-amino acids or methylated amino acids. Preferably, the amino acid denoted Xaa⁶ of Formula IV is a leucine, a serine, or a tyrosine.

In further embodiments, GCC agonist peptides include peptides having the amino acid sequence of Formula V, wherein at least one amino acid of Formula V is a D-amino acid or a methylated amino acid. Preferably, the amino acid at position 16 of Formula V is a D-amino acid or a methylated amino acid. For example, the amino acid at position 16 (i.e., Xaa¹⁶) of Formula V is a d-leucine or a d-serine. Optionally, one or more of the amino acids at position 1-3 of Formula V are D-amino acids or methylated amino acids or a combination of D-amino acids or methylated amino acids. For example, Asn¹, Asp² or Glu³ (or a combination thereof) of Formula V is a D-amino acids or a methylated amino acid. Preferably, the amino acid denoted at Xaa⁶ of Formula V is a leucine, a serine, or a tyrosine.

In additional embodiments, GCRA peptides include peptides having the amino acid sequence of Formula VI, VII-a, VII-b, VIII, or IX. Preferably, the amino acid at position 6 of Formula VI, VII-a, VII-b, VIII, or IX is a leucine, a serine or a tyrosine. In some aspects the amino acid at position 16 of Formula VI, VII-a, VII-b, VIII or IX is a leucine or a serine. Preferably, the amino acid at position 16 of Formula VI, VII-a, VII-b, VIII or IX is a D-amino acid or a methylated amino acid.

In additional embodiments, GCRA peptides include peptides having the amino acid sequence of Formula X, XI, XII, XIII, XIV, XV, XVI or XVII. Optionally, one or more amino acids of Formulas X, XI, XII, XIII, XIV, XV, XVI or XVII are D-amino acids or methylated amino acids. Preferably, the amino acid at the carboxyl terminus of the peptides according to Formulas X, XI, XII, XIII, XIV, XV, XVI or XVII is a D-amino acid or a methylated amino acid. For example the amino acid at the carboxyl terminus of the peptides according to Formulas X, XI, XII, XIII, XIV, XV, XVI or XVII is a D-tyrosine.

Preferably, the amino acid denoted by Xaa⁶ of Formula XIV is a tyrosine, phenylalanine or a serine. Most preferably the amino acid denoted by Xaa⁶ of Formula XIV is a phenylalanine or a serine. Preferably, the amino acid denoted by Xaa⁴ of Formula XV, XVI or XVII is a tyrosine, a phenylalanine, or a serine. Most preferably, the amino acid position Xaa⁴ of Formula XV, XVI or XVII is a phenylalanine or a serine.

In some embodiments, GCRA peptides include peptides containing the amino acid sequence of Formula XVIII. Preferably, the amino acid at position 1 of Formula XVIII is a glutamic acid, aspartic acid, glutamine or lysine. Preferably, the amino acid at position 2 and 3 of Formula XVIII is a glutamic acid, or an aspartic acid. Preferably, the amino acid at position 5 is a glutamic acid. Preferably, the amino acid at position 6 of Formula XVIII is an isoleucine, valine, serine, threonine or tyrosine. Preferably, the amino acid at position 8 of Formula XVIII is a valine or isoleucine. Preferably, the amino acid at position 9 of Formula XVIII is an asparagine. Preferably, the amino acid at position 10 of Formula XVIII is a valine or a methionine. Preferably, the amino acid at position 11 of Formula XVIII is an alanine. Preferably, the amino acid at position 13 of Formula XVIII is a threonine. Preferably, the amino acid at position 14 of Formula XVIII is a glycine. Preferably, the amino acid at position 16 of Formula XVIII is a leucine, serine or threonine

In alternative embodiments, GCRA peptides include peptides containing the amino acid sequence of Formula XIX. Preferably, the amino acid at position 1 of Formula XIX is a serine or asparagine. Preferably, the amino acid at position 2 of Formula XIX is a histidine or an aspartic acid. Preferably, the amino acid at position 3 of Formula XIX is a threonine or a glutamic acid. Preferably, the amino acid at position 5 of Formula XIX is a glutamic acid. Preferably, the amino acid at position 6 of Formula XIX is an isoleucine, leucine, valine or tyrosine. Preferably, the amino acid at position 8, 10, 11, or 13 of Formula XIX is an alanine. Preferably, the amino acid at position 9 of Formula XIX is an asparagine or a phenylalanine. Preferably, the amino acid at position 14 of Formula XIX is a glycine.

In further embodiments, GCRA peptides include peptides containing the amino acid sequence of Formula XX. Preferably, the amino acid at position 1 of Formula XX is a glutamine. Preferably, the amino acid at position 2 or 3 of Formula XX is a glutamic acid or an aspartic acid. Preferably, the amino acid at position 5 of Formula XX is a glutamic acid. Preferably, the amino acid at position 6 of Formula XX is threonine, glutamine, tyrosine, isoleucine, or leucine. Preferably, the amino acid at position 8 of Formula XX is isoleucine or valine. Preferably, the amino acid at position 9 of Formula XX is asparagine. Preferably, the amino acid at position 10 of Formula XX is methionine or valine. Preferably, the amino acid at position 11 of Formula XX is alanine. Preferably, the amino acid at position 13 of Formula XX is a threonine. Preferably, the amino acid at position 1 of Formula XX is a glycine. Preferably, the amino acid at position 15 of Formula XX is a tyrosine. Optionally, the amino acid at position 15 of Formula XX is two-amino acid in length and is Cysteine (Cys), Penicillamine (Pen) homocysteine, or 3-mercaptoproline and serine, leucine or threonine.

In certain embodiments, one or more amino acids of the GCRA peptides can be replaced by a non-naturally occurring amino acid or a naturally or non-naturally occurring amino acid analog. There are many amino acids beyond the standard 20 (Ala, Arg, Asn, Asp, Cys, Gln, Glu, Gly, His, Ile, Leu, Lys, Met, Phe, Pro, Ser, Thr, Trp, Tyr, and Val). Some are naturally-occurring others are not. (See, for example, Hunt, The Non-Protein Amino Acids: In Chemistry and Biochemistry of the Amino Acids, Barrett, Chapman and Hall, 1985). For example, an aromatic amino acid can be replaced by 3,4-dihydroxy-L-phenylalanine, 3-iodo-L-tyrosine, triiodothyronine, L-thyroxine, phenylglycine (Phg) or nor-tyrosine (norTyr). Phg and norTyr and other amino acids including Phe and Tyr can be substituted by, e.g., a halogen, —CH3, —OH, —CH2NH3, —C(O)H, —CH2CH3, —CN, —CH2CH2CH3, —SH, or another group. Any amino acid can be substituted by the D-form of the amino acid.

With regard to non-naturally occurring amino acids or naturally and non-naturally occurring amino acid analogs, a number of substitutions in the polypeptide and agonists described herein are possible alone or in combination.

For example, glutamine residues can be substituted with gamma-Hydroxy-Glu or gamma-Carboxy-Glu. Tyrosine residues can be substituted with an alpha substituted amino acid such as L-alpha-methylphenylalanine or by analogues such as: 3-Amino-Tyr; Tyr(CH3); Tyr(PO3(CH3)2); Tyr(SO3H); beta-Cyclohexyl-Ala; beta-(1-Cyclopentenyl)-Ala; beta-Cyclopentyl-Ala; beta-Cyclopropyl-Ala; beta-Quinolyl-Ala; beta-(2-Thiazolyl)-Ala; beta-(Triazole-1-yl)-Ala; beta-(2-Pyridyl)-Ala; beta-(3-Pyridyl)-Ala; Amino-Phe; Fluoro-Phe; Cyclohexyl-Gly; tBu-Gly; beta-(3-benzothienyl)-Ala; beta-(2-thienyl)-Ala; 5-Methyl-Trp; and A-Methyl-Trp. Proline residues can be substituted with homopro (L-pipecolic acid); hydroxy-Pro; 3,4-Dehydro-Pro; 4-fluoro-Pro; or alpha-methyl-Pro or an N(alpha)-C(alpha) cyclized amino acid analogues with the structure: n=0, 1, 2, 3 Alanine residues can be substituted with alpha-substituted or N-methylated amino acid such as alpha-amino isobutyric acid (aib), L/D-alpha-ethylalanine (L/D-isovaline), L/D-methylvaline, or L/D-alpha-methylleucine or a non-natural amino acid such as beta-fluoro-Ala. Alanine can also be substituted with: n=0, 1, 2, 3 Glycine residues can be substituted with alpha-amino isobutyric acid (aib) or L/D-alpha-ethylalanine (L/D-isovaline).

Further examples of unnatural amino acids include: an unnatural analog of alanine (e.g., L-1-Nal or L-2-Nal); an unnatural analog of tyrosine; an unnatural analogue of glutamine; an unnatural analogue of phenylalanine; an unnatural analogue of serine; an unnatural analogue of threonine; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; an amino acid that is amidated at a site that is not naturally amidated, a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid (e.g., an amino acid containing deuterium, tritium, ¹³C, ¹⁵N, or ¹⁸O); a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; αβ-amino acid; a cyclic amino acid other than proline; an O-methyl-L-tyrosine; an L-3-(2-naphthyl)alanine; a 3-methyl-phenylalanine; a ρ-acetyl-L-phenylalanine; an O-4-allyl-L-tyrosine; a 4-propyl-L-tyrosine; a tri-O-acetyl-GlcNAc β-serine; an L-Dopa; a fluorinated phenylalanine; an isopropyl-L-phenylalanine; a p-azido-L-phenylalanine; a p-acyl-L-phenylalanine; a p-benzoyl-L-phenylalanine; an L-phosphoserine; a phosphonoserine; a phosphonotyrosine; a p-iodo-phenylalanine; a 4-fluorophenylglycine; a p-bromophenylalanine; a p-amino-L-phenylalanine; an isopropyl-L-phenylalanine; L-3-(2-naphthyDalanine; D-3-(2-naphthyl)alanine (dNal); an amino-, isopropyl-, or O-allyl-containing phenylalanine analogue; a dopa, O-methyl-L-tyrosine; a glycosylated amino acid; a p-(propargyloxy)phenylalanine; dimethyl-Lysine; hydroxy-proline; mercaptopropionic acid; methyl-lysine; 3-nitro-tyrosine; norleucine; pyro-glutamic acid; Z (Carbobenzoxyl); ε-Acetyl-Lysine; β-alanine; β-aspartic acid; β-cyclohexylalanine; aminobenzoyl derivative; aminobutyric acid (Abu); citrulline; aminohexanoic acid (Ahx); aminoisobutyric acid (AIB); cyclohexylalanine; d-cyclohexylalanine; cyclohexylglycine; hydroxyproline; nitro-arginine; nitro-phenylalanine; nitro-tyrosine; norvaline; octahydroindole carboxylate; ornithine (Orn); penicillamine (PEN); tetrahydroisoquinoline; diaminobutyric acid; diaminopimelic acid; pyroglutamic acid; homocysteine; homoserine; N-ε-dinitrophenyl-lysine; N-ε-methyl-lysine; N-ε-dimethyl-lysine; N,N,N-ε-trimethyl-lysine; acetamidomethyl protected amino acids and pegylated amino acids. Further examples of unnatural amino acids and amino acid analogs can be found in U.S. 20030108885, U.S. 20030082575, US20060019347 (paragraphs 410-418) and the references cited therein. The polypeptides of the invention can include further modifications including those described in US20060019347, paragraph 589.

“Nal” used herein refers to both L-1-naphthylalanine (L-1-Nal) and L-2-naphthylalanine (L-2-Nal).

In some embodiments, an amino acid can be replaced by a naturally-occurring, non-essential amino acid, e.g., taurine.

Alternatively, the GCRA peptides are cyclic peptides. GCRA cyclic peptides are prepared by methods known in the art. For example, macrocyclization is often accomplished by forming an amide bond between the peptide N- and C-termini, between a side chain and the N- or C-terminus [e.g., with K₃Fe(CN)₆ at pH8.5] (Samson et al., Endocrinology, 137: 5182-5185 (1996)), or between two amino acid side chains, such as cysteine. See, e.g., DeGrado, Adv Protein Chem, 39: 51-124 (1988). In some embodiments, the GCRA peptides of the present invention are bicyclic peptides. In various aspects the GCRA peptides are [4,12; 7,15] bicycles.

In some GCRA peptides one or both members of one or both pairs of Cys residues which normally form a disulfide bond can be replaced by homocysteine, penicillamine, 3-mercaptoproline (Kolodziej et al. 1996 Int J Pept Protein Res 48:274); β,β dimethylcysteine (Hunt et al. 1993 Int J Pept Protein Res 42:249) or diaminopropionic acid (Smith et al. 1978 J Med Chem 2 1:117) to form alternative internal cross-links at the positions of the normal disulfide bonds.

In addition, one or more disulfide bonds can be replaced by alternative covalent cross-links, e.g., an amide linkage (—CH2CH(O)NHCH2- or —CH2NHCH(O)CH2-), an ester linkage, a thioester linkage, a lactam bridge, a carbamoyl linkage, a urea linkage, a thiourea linkage, a phosphonate ester linkage, an alkyl linkage (—CH2CH2CH2CH2-), an alkenyl linkage (—CH2CH═CHCH2-), an ether linkage (—CH2CH2OCH2- or —CH2OCH2CH2-), a thioether linkage (—CH2CH2SCH2- or —CH2SCH2CH2-), an amine linkage (—CH2CH2NHCH2- or —CH2NHCH2CH2-) or a thioamide linkage (—CH2CH(S)HNHCH2- or —CH2NHCH(S)CH2-). For example, Ledu et al. (Proc Nat'l Acad. Sci. 100:11263-78, 2003) describe methods for preparing lactam and amide cross-links. Exemplary GCRA peptides which include a lactam bridge include for example SP-370.

The GCRA peptides can have one or more conventional polypeptide bonds replaced by an alternative bond. Such replacements can increase the stability of the polypeptide. For example, replacement of the polypeptide bond between a residue amino terminal to an aromatic residue (e.g. Tyr, Phe, Trp) with an alternative bond can reduce cleavage by carboxy peptidases and may increase half-life in the digestive tract. Bonds that can replace polypeptide bonds include: a retro-inverso bond (C(O)—NH instead of NH-C(O); a reduced amide bond (NH—CH2); a thiomethylene bond (S—CH2 or CH2-S); an oxomethylene bond (O—CH2 or CH2-O); an ethylene bond (CH2-CH2); a thioamide bond (C(S)—NH); a trans-olefine bond (CH═CH); a fiuoro substituted trans-olefme bond (CF═CH); a ketomethylene bond (C(O)—CHR or CHR—C(O) wherein R is H or CH3; and a fluoro-ketomethylene bond (C(O)—CFR or CFR—C(O) wherein R is H or F or CH3.

The GCRA peptides can be modified using standard modifications. Modifications may occur at the amino (N-), carboxy (C-) terminus, internally or a combination of any of the preceding. In one aspect described herein, there may be more than one type of modification on the polypeptide. Modifications include but are not limited to: acetylation, amidation, biotinylation, cinnamoylation, farnesylation, formylation, myristoylation, palmitoylation, phosphorylation (Ser, Tyr or Thr), stearoylation, succinylation, sulfurylation and cyclisation (via disulfide bridges or amide cyclisation), and modification by Cys3 or Cys5. The GCRA peptides described herein may also be modified by 2,4-dinitrophenyl (DNP), DNP-lysine, modification by 7-Amino-4-methyl-1-coumarin (AMC), flourescein, NBD (7-Nitrobenz-2-Oxa-1,3-Diazole), p-nitro-anilide, rhodamine B, EDANS (5-((2-aminoethyl)amino)naphthalene-1-sulfonic acid), dabcyl, dabsyl, dansyl, texas red, FMOC, and Tamra (Tetramethylrhodamine). The GCRA peptides described herein may also be conjugated to, for example, polyethylene glycol (PEG); alkyl groups (e.g., C1-C20 straight or branched alkyl groups); fatty acid radicals; combinations of PEG, alkyl groups and fatty acid radicals (See, U.S. Pat. No. 6,309,633; Soltero et al., 2001 Innovations in Pharmaceutical Technology 106-110); BSA and KLH (Keyhole Limpet Hemocyanin). The addition of PEG and other polymers which can be used to modify polypeptides of the invention is described in US2006019347 section IX.

In one aspect, the invention provides an Aad-GCRA peptide as taught in, for example, WO20140151206 which is hereby incorporated by reference in its entirety for all purposes. Table 1 of WO20140151206 discloses examples of various alpha-aminoadipic acid derivatives of GCRA Peptides. The Aad-GCRA peptides are analogues uroguanylin, guanylin, lymphoguanylin and ST peptides. Particularly, these analogs contain an a-aminoadipic acid (Ad), preferably at the 3rd position from the N-terminus of each peptide or at the position to the N-terminal side next to the first cysteine (“Cys) residue. For example an Aad-GCRA peptide includes the sequences defined by Formulae I, II, III, IV, V, VI, VII, VII, VIII, IX, XVIII or XXI which can comprise an a-aminoadipic acid.

In some embodiments, the Aad-GCRA peptide is Asn¹-Asp²-Aad³-Cys⁴-Glu⁵-Leu⁶-Cys⁷-Val⁸-Asn⁹-Val¹⁰-Ala¹¹-Cys¹² -Thr¹³-Gly¹⁴-Cys¹⁵-Leu¹⁶ (SEQ ID NO: 251; SP-304-Aad), dAsn¹-Asp²-Aad³-Cys⁴-Glu⁵-Leu⁶-Cys⁷-Val⁸-Asn⁹Val¹⁰-Ala¹¹-Cys¹²-Thr¹³-Gly¹⁴-Cys¹⁵-dLeu^{l6} (SEQ ID NO: 253; SP-333-Aad); Pyglu¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-C¹⁵-dLeu-AMIDE¹⁶ (SEQ ID NO: 254; SP-373-Aad), dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶ (SEQ ID NO: 255; SP-364-Aad); dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dTyr¹⁶ (SEQ ID NO: 256; SP-366-Aad); or Xaaₙ₁-Cys⁴-Xaa⁵-Xaa⁶-Xaa⁷-Xaa⁸-Xaa⁹-Xaa¹⁰-Xaa¹¹-Cys¹²-Xaa¹³-Xaa¹⁴-Xaa¹⁵-Xaaₙ₂¹⁶ (SEQ ID NO: 257).

Also included in the invention are peptides that biologically or functional equivalent to the peptides described herein. The term “biologically equivalent” or functional equivalent” is intended to mean that the compositions of the present invention are capable of demonstrating some or all of the cGMP production modulatory effects.

GCRA peptides can also include derivatives of GCRA peptides which are intended to include extended, truncated, substituted, hybrid, and modified forms of GCRA peptides in which certain amino acids have been deleted or replaced and modifications such as where one or more amino acids have been changed to a modified amino acid or unusual amino acid and modifications such as glycosylation so long the modified form retains the biological activity of GCRA peptides. By retaining the biological activity, it is meant that cGMP and or apoptosis is induced by the GCRA peptide, although not necessarily at the same level of potency as that of a naturally-occurring GCRA peptide identified. In some embodiments, the GCRA peptide is a truncated peptide, where between 1-10 amino acids are deleted and the truncated form retains the biological activity of GCRA peptides. In some embodiments, the GCRA peptide is truncated by about one, about two, about three, about four, about five, about six, about seven, about eight, about nine, or about ten amino acids. In some embodiments, the GCRA peptide is truncated at the N-terminus. In some embodiments, the GCRA peptide is truncated at the C-terminus. In some embodiments, the GCRA peptide is truncated at both the N- and C-termini.

Preferred variants are those that have conservative amino acid substitutions made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a GCRA polypeptide is replaced with another amino acid residue from the same side chain family. Alternatively, in another embodiment, mutations can be introduced randomly along all or part of a GCRA coding sequence, such as by saturation mutagenesis, and the resultant mutants can be screened to identify mutants that retain activity.

TABLE 1
GCRA Peptides (SP-304 and Derivatives)

| | Position | | |
|---|---|---|---|
| Name | of Disulfide bonds | Structure | SEQ ID NO |
| SP-304 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 1 |
| SP-326 | C3:C11, C6:C14 | Asp¹-Glu²-Cys³-Glu⁴-Leu⁵-Cys⁶-Val⁷-Asn⁸-Val⁹-Ala¹⁰-Cys¹¹-Thr¹²-Gly¹³-Cys¹⁴-Leu¹⁵ | 2 |
| SP-327 | C3:C11, C6:C14 | Asp¹-Glu²-Cys³-Glu⁴-Leu⁵-Cys⁶-Val⁷-Asn⁸-Val⁹-Ala¹⁰-Cys¹¹-Thr¹²-Gly¹³-Cys¹⁴ | 3 |
| SP-328 | C2:C10, C5:C13 | Glu¹-Cys²-Glu³-Leu⁴-Cys⁵-Val⁶-Asn⁷-Val⁸-Ala⁹-Cys¹⁰-Thr¹¹-Gly¹²-Cys¹³-Leu¹⁴ | 4 |
| SP-329 | C2:C10, C5:C13 | Glu¹-Cys²-Glu³-Leu⁴-Cys⁵-Val⁶-Asn⁷-Val⁸-Ala⁹-Cys¹⁰-Thr¹¹-Gly¹²-Cys¹³ | 5 |
| SP-330 | C1:C9, C4:C12 | Cys¹-Glu²-Leu³-Cys⁴-Val⁵-Asn⁶-Val⁷-Ala⁸-Cys⁹-Thr¹⁰-Gly¹¹-Cys¹²-Leu¹³ | 6 |
| SP-331 | C1:C9, C4:C12 | Cys¹-Glu²-Leu³-Cys⁴-Val⁵-Asn⁶-Val⁷-Ala⁸-Cys⁹-Thr¹⁰-Gly¹¹-Cys¹² | 7 |
| SP332 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 8 |
| SP-333 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 9 |
| SP-334 | C4:C12, C7:C15 | dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 10 |
| SP-335 | C4:C12, C7:C15 | dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 11 |
| SP-336 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 12 |
| SP-337 | C4:C12, C7:C15 | dAsn¹-Asp²-Glu³-Cys⁴-Glu⁵-dLeu⁶-Cys⁷-Val⁸-Asn⁹-Val¹⁰-Ala¹¹-Cys¹²-Thr¹³-Gly¹⁴-Cys¹⁵-dLeu¹⁶ | 13 |
| SP-338 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵ | 14 |
| SP-342 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 15 |
| SP-343 | C4:C12, C7:C15 | PEG3-dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 16 |
| SP-344 | C4:C12, C7:C15 | PEG3-dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 17 |
| SP-347 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 18 |
| SP-348 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 19 |
| SP-350 | C4:C12, C7:C15 | PEG3-dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 20 |
| SP-352 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 21 |
| SP-358 | C4:C12, C7:C15 | PEG3-dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 22 |
| SP-359 | C4:C12, C7:C15 | PEG3-dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 23 |
| SP-360 | C4:C12, C7:C15 | dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 24 |
| SP-361 | C4:C12, C7:C15 | dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 25 |
| SP-362 | C4:C12, C7:C15 | PEG3-dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 26 |
| SP-368 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dNal¹⁶ | 27 |
| SP-369 | C4:C12, C7:C15 | dAsn¹-Asp²-Glu³-Cys⁴-Glu⁵-Leu⁶-Cys⁷-AIB⁸-Asn⁹-AIB¹⁰-Ala¹¹-Cys¹²-Thr¹³-Gly¹⁴-Cys¹⁵-dLeu¹⁶ | 28 |
| SP-370 | C4:C12, 7:15 | dAsn¹-Asp²-Glu³-Cys⁴-Glu⁵-Leu⁶-Asp[Lactam]⁷-Val⁸-Asn⁹-Val¹⁰-Ala¹¹-Cys¹²-Thr¹³-Gly¹⁴-Orn¹⁵-dLeu¹ | 29 |
| SP-371 | C4:C12, C7:C15 | dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 30 |
| SP-372 | C4:C12, C7:C15 | dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 31 |
| N1 | C4:C12, C7:C15 | PEG3-dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 32 |
| N2 | C4:C12, C7:C15 | PEG3-dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 33 |
| N3 | C4:C12, C7:C15 | dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 34 |
| N4 | C4:C12, C7:C15 | PEG3-dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 35 |
| N5 | C4:C12, C7:C15 | PEG3-dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 36 |
| N6 | C4:C12, C7:C15 | dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 37 |
| N7 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 38 |
| N8 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 39 |
| N9 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 40 |
| N10 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 41 |
| N11 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 42 |
| N12 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶ | 43 |
| N13 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 44 |
| Formula I | C4:C12, C7:C15 | Asn¹-Asp²-Glu³-Cys⁴-Xaa⁵-Xaa⁶-Cys⁷-Xaa⁸-Xaa⁹-Xaa¹⁰-Xaa¹¹-Cys¹²-Xaa¹³-Xaa¹⁴-Cys¹⁵-Xaa¹⁶ | 45 |
| Formula II | C4:C12, C7:C15 | Xaaₙ₁-Cys⁴-Xaa⁵-Xaa⁶-Cys⁷-Xaa⁸-Xaa⁹-Xaa¹⁰-Xaa¹¹-Cys¹²-Xaa¹³-Xaa¹⁴-Cys¹⁵-Xaaₙ₂¹⁶ | 46 |
| Formula III | 4:12, 7:15 | Xaaₙ₁-Maa⁴-Glu⁵-Xaa⁶-Maa⁷-Val⁸-Asn⁹-Val¹⁰-Ala¹¹-Maa¹²-Thr¹³-Gly¹⁴-Maa¹⁵-Xaaₙ₂ | 47 |
| Formula IV | 4:12, 7:15 | Xaaₙ₁-Maa⁴-Xaa⁵-Xaa⁶-Maa⁷-Xaa⁸-Xaa⁹-Xaa¹⁰-Xaa¹¹-Maa¹²-Xaa¹³-Xaa¹⁴-Maa¹⁵-Xaaₙ₂ | 48 |
| Formula V | C4:C12, C7:C15 | Asn¹-Asp²-Asp³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-Xaa¹⁶ | 49 |
| Formula VI | C4:C12, C7:C15 | dAsn¹-Glu²-Glu³-Cys⁴-Xaa⁵-Xaa⁶-Cys⁷-X3⁸-Asn⁹-Xaa¹⁰-Xaa¹¹-Cys¹²-Xaa¹³-Xaa¹⁴-Cys¹⁵-d-Xaa¹⁶ | 50 |
| Formula VII-a | C4:C12, C7:C15 | dAsn¹-dGlu²-Asp³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 51 |
| Formula VII-b | C4:C12, C7:C15 | dAsn¹-dAsp²-Glu³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 52 |
| Formula VIII | C4:C12, C7:C15 | dAsn¹-dAsp²-dGlu³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 53 |
| Formula IX | C4:C12, C7:C15 | dAsn¹-dGlu²-dGlu³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 54 |
| Formula XXI | C4:C12, C7:C15 | Xaaₙ₁-Cys⁴-Xaa⁵-Xaa⁶-Xaa⁷-Xaa⁸-Xaa⁹-Xaa¹⁰-Xaa¹¹-Cys¹²-Xaa¹³-Xaa¹⁴-Xaa¹⁵-Xaaₙ₂¹⁶ | 250 |

TABLE 2
Linaclotide and Derivatives

| | Position of Disulfide | | SEQ ID |
|---|---|---|---|
| Name | Bonds | Structure | NO: |
| SP- | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴ | 55 |
| 339(linaclotide) | | | |
| SP-340 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³ | 56 |
| SP-349 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴-PEG3 | 57 |
| SP-353 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵-Tyr¹⁶ | 58 |
| SP-354 | C3:C8, C4:C12, C7:C15 | Asn¹-Phe²⁻Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵-Tyr¹⁶ | 59 |
| SP-355 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-dTyr¹⁴ | 60 |
| SP-357 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴ | 61 |
| SP-374 | C3:C8, C4:C12, C7:C15 | Asn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵-Tyr¹⁶ | 62 |
| SP-375 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵-dTyr¹⁶ | 63 |
| SP-376 | C3:C8, C4:C12, C7:C15 | dAsn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵-Tyr¹⁶ | 64 |
| SP-377 | C3:C8, C4:C12, C7:C15 | dAsn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵-dTyr¹⁶ | 65 |
| SP-378 | C3:C8, C4:C12, C7:C15 | Asn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵-dTyr¹⁶ | 66 |
| SP-379 | C3:C8, C4:C12, C7:C15 | dAsn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵-Tyr¹⁶ | 67 |
| SP-380 | C3:C8, C4:C12, C7:C15 | dAsn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵-dTyr¹⁶ | 68 |
| SP-381 | C3:C8, C4:C12, C7:15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵-dTyr¹⁶ | 69 |
| SP-382 | C3:C8, C4:C12, C7:15 | dAsn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵-Tyr¹⁶ | 70 |
| SP-383 | C3:C8, C4:C12, C7:15 | dAsn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵-dTyr¹⁶ | 71 |
| SP384 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴-PEG3 | 72 |
| N14 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-PEG3 | 73 |
| N15 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³ | 74 |
| N16 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-PEG3 | 75 |
| N17 | C3:C8, C4:C12, C7:C15 | PEG3-Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵-Tyr¹⁶-PEG3 | 76 |
| N18 | C3:C8, C4:C12, C7:C15 | PEG3-Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵-Tyr¹⁶ | 77 |
| N19 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵-Tyr¹⁶-PEG3 | 78 |
| N20 | C3:C8, C4:C12, C7:C15 | PEG3-Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵-Tyr¹⁶-PEG3 | 79 |
| N21 | C3:C8, C4:C12, C7:C15 | PEG3-Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵-Tyr¹⁶ | 80 |
| N22 | C3:C8, C4:C12, C7:C15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵-Tyr¹⁶-PEG3 | 81 |
| N23 | C3:C8, C4:C12, C7:C15 | PEG3-Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴-Cys¹⁵-Tyr¹⁶-PEG3 | 82 |
| N24 | C3:C8, C4:C12, C7:C15 | PEG3-Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴-Cys¹⁵-Tyr¹⁶ | 83 |
| N25 | C3:C8, C4:C12, C7:C15 | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴-Cys¹⁵-Tyr¹⁶-PEG3 | 84 |
| N26 | C1:C6, C2:C10, C5:C13 | Cys¹-Cys²-Glu3-Ser⁴-Cys⁵-Cys⁶-Asn⁷-Pro⁸-Ala⁹-Cys¹⁰-Thr¹¹-Gly¹²-Cys¹³-Tyr¹⁴ | 85 |
| N27 | C1:C6, C2:C10, C5:C13 | Cys¹-Cys²-Glu3-Phe⁴-Cys⁵-Cys⁶-Asn⁷-Pro⁸-Ala⁹-Cys¹⁰-Thr¹¹-Gly¹²-Cys¹³-Tyr¹⁴ | 86 |
| N28 | C1:C6, C2:C10, C5:C13 | Cys¹-Cys²-Glu3-Ser⁴-Cys⁵-Cys⁶-Asn⁷-Pro⁸-Ala⁹-Cys¹⁰-Thr¹¹-Gly¹²-Cys¹³- | 87 |
| N29 | C1:C6, C2:C10, C5:C13 | Cys¹-Cys²-Glu3-Phe⁴-Cys⁵-Cys⁶-Asn⁷-Pro⁸-Ala⁹-Cys¹⁰-Thr¹¹-Gly¹²-Cys¹³ | 88 |
| N30 | 1:6, 2:10, 5:13 | Pen¹-Pen²-Glu3-Tyr⁴-Pen⁵-Pen⁶-Asn⁷-Pro⁸-Ala⁹-Pen¹⁰-Thr¹¹-Gly¹²-Pen¹³-Tyr¹⁴ | 89 |
| N31 | 1:6, 2:10, 5:13 | Pen¹-Pen²-Glu3-Tyr⁴-Pen⁵-Pen⁶-Asn⁷-Pro⁸-Ala⁹-Pen¹⁰-Thr¹¹-Gly¹²-Pen¹³ | 90 |
| Formula X | C9:C14, C10:C18, | Xaa¹-Xaa²⁻Xaa³-Xaa⁴-Xaa⁵-Xaa⁶-Asn⁷-Tyr⁸-Cys⁹-Cys¹⁰-Xaa¹¹-Tyr¹²-Cys¹³-Cys¹⁴-Xaa¹⁵-Xaa¹⁶-Xaa¹⁷- | 91 |
| | C13:C21 | Cys¹⁸-Xaa¹⁹-Xaa²⁰-Cys²¹-Xaa²² | |
| Formula | C9:C14, C10:C18, | Xaa¹-Xaa²⁻Xaa³-Xaa⁴-Xaa⁵-Xaa⁶-Asn⁷-Phe⁸-Cys⁹-Cys¹⁰-Xaa¹¹-Phe¹²-Cys¹³-Cys¹⁴-Xaa¹⁵-Xaa¹⁶-Xaa¹⁷- | 92 |
| XI | C13:C21 | Cys¹⁸-Xaa¹⁹-Xaa²⁰-Cys²¹-Xaa²² | |
| Formula XII | C3:C8, C4:C12, C7:C15 | Asn¹-Phe²-Cys³-Cys⁴-Xaa⁵-Phe⁶-Cys⁷-Cys⁸-Xaa⁹-Xaa¹⁰-Xaa¹¹⁻Cys¹²-Xaa³-Xaa¹⁴⁻Cys¹⁵-Xaa¹⁶ | 93 |
| Formula XIII | 3:8, 4:12, 7:15 | Asn¹-Phe²-Pen³-Cys⁴-Xaa⁵-Phe⁶-Cys⁷⁻Pen⁸-Xaa⁹-Xaa¹⁰-Xaa¹¹⁻Cys¹²-Xaa¹³-Xaa¹⁴⁻Cys¹⁵-Xaa¹⁶ | 94 |
| Formula XIV | 3:8, 4:12, 7:15 | Asn¹-Phe²-Maa³-Maa⁴-Xaa⁵-Xaa⁶-Maa⁷⁻Maa⁸-Xaa⁹-Xaa¹⁰-Xaa¹¹⁻Maa¹²-Xaa¹³-Xaa¹⁴⁻Maa¹⁵-Xaa¹⁶ | 95 |
| Formula XV | 1:6, 2:10, 5:13 | Maa¹-Maa²-Glu3-Xaa⁴-Maa⁵-Maa⁶-Asn⁷-Pro⁸-Ala⁹-Maa¹⁰-Thr¹¹-Gly¹²-Maa¹³-Tyr¹⁴ | 96 |
| Formula XVI | 1:6, 2:10, 5:13 | Maa¹-Maa²-Glu3-Xaa⁴-Maa⁵-Maa⁶-Asn⁷-Pro⁸-Ala⁹-Maa¹⁰-Thr¹¹-Gly¹²-Maa¹³ | 97 |
| Formula XVII | 1:6, 2:10, 5:13 | Xaaₙ₃-Maa¹-Maa²-Xaa³-Xaa⁴-Maa⁵-Maa⁶-Xaa⁷-Xaa⁸-Xaa⁹-Maa¹⁰-Xaa¹¹-Xaa¹²-Maa¹³-Xaaₙ₂ | 98 |

TABLE 3
GCRA Peptides

| | | | SEQ |
|---|---|---|---|
| | Position of | | ID |
| Name | Disulfide bonds | Structure | NO: |
| SP-363 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 99 |
| | | Gly¹⁴-Cys¹⁵-dLeu-AMIDE¹⁶ | |
| SP-364 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 100 |
| | | Gly¹⁴-Cys¹⁵-dSer¹⁶ | |
| SP-365 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 101 |
| | | Gly¹⁴-Cys¹⁵-dSer-AMIDE¹⁶ | |
| SP-366 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 102 |
| | | Gly¹⁴-Cys¹⁵-dTyr¹⁶ | |
| SP-367 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 103 |
| | | Gly¹⁴-Cys¹⁵-dTyr-AMIDE¹⁶ | |
| SP-373 | C4:C12, C7:C15 | Pyglu¹-Asp²-Glu³-Cys⁴-Glu⁵-Leu⁶-Cys⁷-Val⁸-Asn⁹-Val¹⁰-Ala¹¹-Cys¹²-Thr¹³- | 104 |
| | | Gly¹⁴-Cys¹⁵-dLeu-AMIDE¹⁶ | |
| / | C4:C12, C7:C15 | Pyglu¹-Asp²-Glu³-Cys⁴-Glu⁵-Leu⁶-Cys⁷-Val⁸-Asn⁹-Val¹⁰-Ala¹¹-Cys¹²-Thr¹³- | 251 |
| | | Gly¹⁴-Cys¹⁵-Leu¹⁶ | |
| SP- | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³- | 105 |
| 304diPEG | | Gly¹⁴-Cys¹⁵-Leu¹⁶-PEG3 | |
| SP-304N- | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³- | 106 |
| PEG | | Gly¹⁴-Cys¹⁵-Leu¹⁶ | |
| SP-304C- | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³- | 107 |
| PEG | | Gly¹⁴-Cys¹⁵-Leu¹⁶-PEG3 | |

TABLE 4
SP-304 Analogs, Uroguanylin, and Uroguanylin Analogs

| | Position of | | |
|---|---|---|---|
| | Disulfide | | SEQ ID |
| Name | bonds | Structure | NO |
| Formula | C4:C12, | Xaa¹-Xaa²-Xaa³-Maa⁴-Xaa⁵-Xaa⁶-Maa⁷-Xaa⁸-Xaa⁹-Xaa¹⁰-Xaa¹¹-Maa¹²-Xaa¹³- | 108 |
| XVIII | C7:C15 | Xaa¹⁴-Maa¹⁵-Xaa¹⁶ | |
| Uroguanylin | C4:C12, | Asn¹-Asp²-Asp³-Cys⁴-Glu⁵-Leu⁶-Cys⁷-Val⁸-Asn⁹-Val¹⁰-Ala¹¹-Cys¹²-Thr¹³- | 109 |
| | C7:C15 | Gly¹⁴-Cys¹⁵-Leu¹⁶ | |
| N32 | C4:C12, C7:C15 | Glu¹-Asp²-Asp³-Cys⁴-Glu⁵-Leu⁶-Cys⁷-Val⁸-Asn⁹-Val¹⁰-Ala¹¹-Cys¹²-Thr¹³- | 110 |
| | | Gly¹⁴-Cys¹⁵-Leu¹⁶ | |
| N33 | C4:C12, C7:C15 | Glu¹-Asp²-Glu³-Cys⁴-Glu⁵-Leu⁶-Cys⁷-Val⁸-Asn⁹-Val¹⁰-Ala¹¹-Cys¹²-Thr¹³- | 111 |
| | | Gly¹⁴-Cys¹⁵-Leu¹⁶ | |
| N34 | C4:C12, C7:C15 | Glu¹-Glu²-Asp³-Cys⁴-Glu⁵-Leu⁶-Cys⁷-Val⁸-Asn⁹-Val¹⁰-Ala¹¹-Cys¹²-Thr¹³- | 112 |
| | | Gly¹⁴-Cys¹⁵-Leu¹⁶ | |
| N35 | C4:C12, C7:C15 | Glu¹-Glu²-Glu³-Cys⁴-Glu⁵-Leu⁶-Cys⁷-Val⁸-Asn⁹-Val¹⁰-Ala¹¹-Cys¹²-Thr¹³- | 113 |
| | | Gly¹⁴-Cys¹⁵-Leu¹⁶ | |
| N36 | C4:C12, C7:C15 | Asp¹-Asp²-Asp³-Cys⁴-Glu⁵-Leu⁶-Cys⁷-Val⁸-Asn⁹-Val¹⁰-Ala¹¹-Cys¹²-Thr¹³- | 114 |
| | | Gly¹⁴-Cys¹⁵-Leu¹⁶ | |
| N37 | C4:C12, C7:C15 | Asp¹-Asp²-Glu³-Cys⁴-Glu⁵-Leu⁶-Cys⁷-Val⁸-Asn⁹-Val¹⁰-Ala¹¹-Cys¹²-Thr¹³- | 115 |
| | | Gly¹⁴-Cys¹⁵-Leu¹⁶ | |
| N38 | C4:C12, C7:C15 | Asp¹-Glu²-Asp³-Cys⁴-Glu⁵-Leu⁶-Cys⁷-Val⁸-Asn⁹-Val¹⁰-Ala¹¹-Cys¹²-Thr¹³- | 116 |
| | | Gly¹⁴-Cys¹⁵-Leu¹⁶ | |
| N39 | C4:C12, C7:C15 | Asp¹-Glu²-Glu³-Cys⁴-Glu⁵-Leu⁶-Cys⁷-Val⁸-Asn⁹-Val¹⁰-Ala¹¹-Cys¹²-Thr¹³- | 117 |
| | | Gly¹⁴-Cys¹⁵-Leu¹⁶ | |
| N40 | C4:C12, C7:C15 | Gln¹-Asp²-Asp³-Cys⁴-Glu⁵-Leu⁶-Cys⁷-Val⁸-Asn⁹-Val¹⁰-Ala¹¹-Cys¹²-Thr¹³- | 118 |
| | | Gly¹⁴-Cys¹⁵-Leu¹⁶ | |
| N41 | C4:C12, C7:C15 | Gln¹-Asp²-Glu³-Cys⁴-Glu⁵-Leu⁶-Cys⁷-Val⁸-Asn⁹-Val¹⁰-Ala¹¹-Cys¹²-Thr¹³- | 119 |
| | | Gly¹⁴-Cys¹⁵-Leu¹⁶ | |
| N42 | C4:C12, C7:C15 | Gln¹-Glu²-Asp³-Cys⁴-Glu⁵-Leu⁶-Cys⁷-Val⁸-Asn⁹-Val¹⁰-Ala¹¹-Cys¹²-Thr¹³- | 120 |
| | | Gly¹⁴-Cys¹⁵-Leu¹⁶ | |
| N43 | C4:C12, C7:C15 | Gln¹-Glu²-Glu³-Cys⁴-Glu⁵-Leu⁶-Cys⁷-Val⁸-Asn⁹-Val¹⁰-Ala¹¹-Cys¹²-Thr¹³- | 121 |
| | | Gly¹⁴-Cys¹⁵-Leu¹⁶ | |
| N44 | C4:C12, C7:C15 | Lys¹-Asp²-Asp³-Cys⁴-Glu⁵-Leu⁶-Cys⁷-Val⁸-Asn⁹-Val¹⁰-Ala¹¹-Cys¹²-Thr¹³- | 122 |
| | | Gly¹⁴-Cys¹⁵-Leu¹⁶ | |
| N45 | C4:C12, C7:C15 | Lys¹-Asp²-Glu³-Cys⁴-Glu⁵-Leu⁶-Cys⁷-Val⁸-Asn⁹-Val¹⁰-Ala¹¹-Cys¹²-Thr¹³- | 123 |
| | | Gly¹⁴-Cys¹⁵-Leu¹⁶ | |
| N46 | C4:C12, C7:C15 | Lys¹-Glu²-Asp³-Cys⁴-Glu⁵-Leu⁶-Cys⁷-Val⁸-Asn⁹-Val¹⁰-Ala¹¹-Cys¹²-Thr¹³- | 124 |
| | | Gly¹⁴-Cys¹⁵-Leu¹⁶ | |
| N47 | C4:C12, C7:C15 | Lys¹-Glu²-Glu³-Cys⁴-Glu⁵-Leu⁶-Cys⁷-Val⁸-Asn⁹-Val¹⁰-Ala¹¹-Cys¹²-Thr¹³- | 125 |
| | | Gly¹⁴-Cys¹⁵-Leu¹⁶ | |
| N48 | C4:C12, C7:C15 | Glu¹-Asp²-Asp³-Cys⁴-Glu⁵-Leu⁶-Cys⁷-Val⁸-Asn⁹-Val¹⁰-Ala¹¹-Cys¹²-Thr¹³- | 126 |
| | | Gly¹⁴-Cys¹⁵-Ser¹⁶ | |
| N49 | C4:C12, C7:C15 | Glu¹-Asp²-Glu³-Cys⁴-Glu⁵-Leu⁶-Cys⁷-Val⁸-Asn⁹-Val¹⁰-Ala¹¹-Cys¹²-Thr¹³- | 127 |
| | | Gly¹⁴-Cys¹⁵-Ser¹⁶ | |
| N50 | C4:C12, C7:C15 | Glu¹-Glu²-Asp³-Cys⁴-Glu⁵-Leu⁶-Cys⁷-Val⁸-Asn⁹-Val¹⁰-Ala¹¹-Cys¹²-Thr¹³- | 128 |
| | | Gly¹⁴-Cys¹⁵-Ser¹⁶ | |
| N51 | C4:C12, C7:C15 | Glu¹-Glu²-Glu³-Cys⁴-Glu⁵-Leu⁶-Cys⁷-Val⁸-Asn⁹-Val¹⁰-Ala¹¹-Cys¹²-Thr¹³- | 129 |
| | | Gly¹⁴-Cys¹⁵-Ser¹⁶ | |
| N52 | C4:C12, C7:C15 | Asp¹-Asp²-Asp³-Cys⁴-Glu⁵-Leu⁶-Cys⁷-Val⁸-Asn⁹-Val¹⁰-Ala¹¹-Cys¹²-Thr¹³- | 130 |
| | | Gly¹⁴-Cys¹⁵-Ser¹⁶ | |
| N53 | C4:C12, C7:C15 | Asp¹-Asp²-Glu³-Cys⁴-Glu⁵-Leu⁶-Cys⁷-Val⁸-Asn⁹-Val¹⁰-Ala¹¹-Cys¹²-Thr¹³- | 131 |
| | | Gly¹⁴-Cys¹⁵-Ser¹⁶ | |
| N54 | C4:C12, C7:C15 | Asp¹-Glu²-Asp³-Cys⁴-Glu⁵-Leu⁶-Cys⁷-Val⁸-Asn⁹-Val¹⁰-Ala¹¹-Cys¹²-Thr¹³- | 132 |
| | | Gly¹⁴-Cys¹⁵-Ser¹⁶ | |
| N55 | C4:C12, C7:C15 | Asp¹-Glu²-Glu³-Cys⁴-Glu⁵-Leu⁶-Cys⁷-Val⁸-Asn⁹-Val¹⁰-Ala¹¹-Cys¹²-Thr¹³- | 133 |
| | | Gly¹⁴-Cys¹⁵-Ser¹⁶ | |
| N56 | C4:C12, C7:C15 | Gln¹-Asp²-Asp³-Cys⁴-Glu⁵-Leu⁶-Cys⁷-Val⁸-Asn⁹-Val¹⁰-Ala¹¹-Cys¹²-Thr¹³- | 134 |
| | | Gly¹⁴-Cys¹⁵-Ser¹⁶ | |
| N57 | C4:C12, C7:C15 | Gln¹-Asp²-Glu³-Cys⁴-Glu⁵-Leu⁶-Cys⁷-Val⁸-Asn⁹-Val¹⁰-Ala¹¹-Cys¹²-Thr¹³- | 135 |
| | | Gly¹⁴-Cys¹⁵-Ser¹⁶ | |
| N58 | C4:C12, C7:C15 | Gln¹-Glu²-Asp³-Cys⁴-Glu⁵-Leu⁶-Cys⁷-Val⁸-Asn⁹-Val¹⁰-Ala¹¹-Cys¹²-Thr¹³- | 136 |
| | | Gly¹⁴-Cys¹⁵-Ser¹⁶ | |
| N59 | C4:C12, C7:C15 | Gln¹-Glu²-Glu³-Cys⁴-Glu⁵-Leu⁶-Cys⁷-Val⁸-Asn⁹-Val¹⁰-Ala¹¹-Cys¹²-Thr¹³- | 137 |
| | | Gly¹⁴-Cys¹⁵-Ser¹⁶ | |
| N60 | C4:C12, C7:C15 | Lys¹-Asp²-Asp³-Cys⁴-Glu⁵-Leu⁶-Cys⁷-Val⁸-Asn⁹-Val¹⁰-Ala¹¹-Cys¹²-Thr¹³- | 138 |
| | | Gly¹⁴-Cys¹⁵-Ser¹⁶ | |
| N61 | C4:C12, C7:C15 | Lys¹-Asp²-Glu³-Cys⁴-Glu⁵-Leu⁶-Cys⁷-Val⁸-Asn⁹-Val¹⁰-Ala¹¹-Cys¹²-Thr¹³- | 139 |
| | | Gly¹⁴-Cys¹⁵-Ser¹⁶ | |
| N62 | C4:C12, C7:C15 | Lys¹-Glu²-Asp³-Cys⁴-Glu⁵-Leu⁶-Cys⁷-Val⁸-Asn⁹-Val¹⁰-Ala¹¹-Cys¹²-Thr¹³- | 140 |
| | | Gly¹⁴-Cys¹⁵-Ser¹⁶ | |
| N63 | C4:C12, C7:C15 | Lys¹-Glu²-Glu³-Cys⁴-Glu⁵-Leu⁶-Cys⁷-Val⁸-Asn⁹-Val¹⁰-Ala¹¹-Cys¹²-Thr¹³- | 141 |
| | | Gly¹⁴-Cys¹⁵-Ser¹⁶ | |
| N65 | C4:C12, C7:C15 | Glu¹-Asp²-Asp³-Cys⁴-Glu⁵-Leu⁶-Cys⁷-Ile⁸-Asn⁹-Met¹⁰-Ala¹¹-Cys¹²-Thr¹³- | 142 |
| | | Gly¹⁴-Cys¹⁵-Leu¹⁶ | |
| N66 | C4:C12, C7:C15 | Glu¹-Asp²-Glu³-Cys⁴-Glu⁵-Leu⁶-Cys⁷-Ile⁸-Asn⁹-Met¹⁰-Ala¹¹-Cys¹²-Thr¹³- | 143 |
| | | Gly¹⁴-Cys¹⁵-Leu¹⁶ | |
| N67 | C4:C12, C7:C15 | Glu¹-Glu²-Asp³-Cys⁴-Glu⁵-Leu⁶-Cys⁷-Ile⁸-Asn⁹-Met¹⁰-Ala¹¹-Cys¹²-Thr¹³- | 144 |
| | | Gly¹⁴-Cys¹⁵-Leu¹⁶ | |
| N68 | C4:C12, C7:C15 | Glu¹-Glu²-Glu³-Cys⁴-Glu⁵-Leu⁶-Cys⁷-Ile⁸-Asn⁹-Met¹⁰-Ala¹¹-Cys¹²-Thr¹³- | 145 |
| | | Gly¹⁴-Cys¹⁵-Leu¹⁶ | |
| N69 | C4:C12, C7:C15 | Asp¹-Asp²-Asp³-Cys⁴-Glu⁵-Leu⁶-Cys⁷-Ile⁸-Asn⁹-Met¹⁰-Ala¹¹-Cys¹²-Thr¹³- | 146 |
| | | Gly¹⁴-Cys¹⁵-Leu¹⁶ | |
| N70 | C4:C12, C7:C15 | Asp¹-Asp²-Glu³-Cys⁴-Glu⁵-Leu⁶-Cys⁷-Ile⁸-Asn⁹-Met¹⁰-Ala¹¹-Cys¹²-Thr¹³- | 147 |
| | | Gly¹⁴-Cys¹⁵-Leu¹⁶ | |
| N71 | C4:C12, C7:C15 | Asp¹-Glu²-Asp³-Cys⁴-Glu⁵-Leu⁶-Cys⁷-Ile⁸-Asn⁹-Met¹⁰-Ala¹¹-Cys¹²-Thr¹³- | 148 |
| | | Gly¹⁴-Cys¹⁵-Leu¹⁶ | |
| N72 | C4:C12, C7:C15 | Asp¹-Glu²-Glu³-Cys⁴-Glu⁵-Leu⁶-Cys⁷-Ile⁸-Asn⁹-Met¹⁰-Ala¹¹-Cys¹²-Thr¹³- | 149 |
| | | Gly¹⁴-Cys¹⁵-Leu¹⁶ | |
| N73 | C4:C12, C7:C15 | Gln¹-Asp²-Asp³-Cys⁴-Glu⁵-Leu⁶-Cys⁷-Ile⁸-Asn⁹-Met¹⁰-Ala¹¹-Cys¹²-Thr¹³- | 150 |
| | | Gly¹⁴-Cys¹⁵-Leu¹⁶ | |
| N74 | C4:C12, C7:C15 | Gln¹-Asp²-Glu³-Cys⁴-Glu⁵-Leu⁶-Cys⁷-Ile⁸-Asn⁹-Met¹⁰-Ala¹¹-Cys¹²-Thr¹³- | 151 |
| | | Gly¹⁴-Cys¹⁵-Leu¹⁶ | |
| N75 | C4:C12, C7:C15 | Gln¹-Glu²-Asp³-Cys⁴-Glu⁵-Leu⁶-Cys⁷-Ile⁸-Asn⁹-Met¹⁰-Ala¹¹-Cys¹²-Thr¹³- | 152 |
| | | Gly¹⁴-Cys¹⁵-Leu¹⁶ | |
| N76 | C4:C12, C7:C15 | Gln¹-Glu²-Glu³-Cys⁴-Glu⁵-Leu⁶-Cys⁷-Ile⁸-Asn⁹-Met¹⁰-Ala¹¹-Cys¹²-Thr¹³- | 153 |
| | | Gly¹⁴-Cys¹⁵-Leu¹⁶ | |
| N77 | C4:C12, C7:C15 | Lys¹-Asp²-Asp³-Cys⁴-Glu⁵-Leu⁶-Cys⁷-Ile⁸-Asn⁹-Met¹⁰-Ala¹¹-Cys¹²-Thr¹³- | 154 |
| | | Gly¹⁴-Cys¹⁵-Leu¹⁶ | |
| N78 | C4:C12, C7:C15 | Lys¹-Asp²-Glu³-Cys⁴-Glu⁵-Leu⁶-Cys⁷-Ile⁸-Asn⁹-Met¹⁰-Ala¹¹-Cys¹²-Thr¹³- | 155 |
| | | Gly¹⁴-Cys¹⁵-Leu¹⁶ | |
| N79 | C4:C12, C7:C15 | Lys¹-Glu²-Asp³-Cys⁴-Glu⁵-Leu⁶-Cys⁷-Ile⁸-Asn⁹-Met¹⁰-Ala¹¹-Cys¹²-Thr¹³- | 156 |
| | | Gly¹⁴-Cys¹⁵-Leu¹⁶ | |
| N80 | C4:C12, C7:C15 | Lys¹-Glu²-Glu³-Cys⁴-Glu⁵-Leu⁶-Cys⁷-Ile⁸-Asn⁹-Met¹⁰-Ala¹¹-Cys¹²-Thr¹³- | 157 |
| | | Gly¹⁴-Cys¹⁵-Leu¹⁶ | |
| N81 | C4:C12, C7:C15 | Glu¹-Asp²-Asp³-Cys⁴-Glu⁵-Leu⁶-Cys⁷-Ile⁸-Asn⁹-Met¹⁰-Ala¹¹-Cys¹²-Thr¹³- | 158 |
| | | Gly¹⁴-Cys¹⁵-Ser¹⁶ | |
| N82 | C4:C12, C7:C15 | Glu¹-Asp²-Glu³-Cys⁴-Glu⁵-Leu⁶-Cys⁷-Ile⁸-Asn⁹-Met¹⁰-Ala¹¹-Cys¹²-Thr¹³- | 159 |
| | | Gly¹⁴-Cys¹⁵-Ser¹⁶ | |
| N83 | C4:C12, C7:C15 | Glu¹-Glu²-Asp³-Cys⁴-Glu⁵-Leu⁶-Cys⁷-Ile⁸-Asn⁹-Met¹⁰-Ala¹¹-Cys¹²-Thr¹³- | 160 |
| | | Gly¹⁴-Cys¹⁵-Ser¹⁶ | |
| N84 | C4:C12, C7:C15 | Glu¹-Glu²-Glu³-Cys⁴-Glu⁵-Leu⁶-Cys⁷-Ile⁸-Asn⁹-Met¹⁰-Ala¹¹-Cys¹²-Thr¹³- | 161 |
| | | Gly¹⁴-Cys¹⁵-Ser¹⁶ | |
| N85 | C4:C12, C7:C15 | Asp¹-Asp²-Asp³-Cys⁴-Glu⁵-Leu⁶-Cys⁷-Ile⁸-Asn⁹-Met¹⁰-Ala¹¹-Cys¹²-Thr¹³- | 162 |
| | | Gly¹⁴-Cys¹⁵-Ser¹⁶ | |
| N86 | C4:C12, C7:C15 | Asp¹-Asp²-Glu³-Cys⁴-Glu⁵-Leu⁶-Cys⁷-Ile⁸-Asn⁹-Met¹⁰-Ala¹¹-Cys¹²-Thr¹³- | 163 |
| | | Gly¹⁴-Cys¹⁵-Ser¹⁶ | |
| N87 | C4:C12, C7:C15 | Asp¹-Glu²-Asp³-Cys⁴-Glu⁵-Leu⁶-Cys⁷-Ile⁸-Asn⁹-Met¹⁰-Ala¹¹-Cys¹²-Thr¹³- | 164 |
| | | Gly¹⁴-Cys¹⁵-Ser¹⁶ | |
| N88 | C4:C12, C7:C15 | Asp¹-Glu²-Glu³-Cys⁴-Glu⁵-Leu⁶-Cys⁷-Ile⁸-Asn⁹-Met¹⁰-Ala¹¹-Cys¹²-Thr¹³- | 165 |
| | | Gly¹⁴-Cys¹⁵-Ser¹⁶ | |
| N89 | C4:C12, C7:C15 | Gln¹-Asp²-Asp³-Cys⁴-Glu⁵-Leu⁶-Cys⁷-Ile⁸-Asn⁹-Met¹⁰-Ala¹¹-Cys¹²-Thr¹³- | 166 |
| | | Gly¹⁴-Cys¹⁵-Ser¹⁶ | |
| N90 | C4:C12, C7:C15 | Gln¹-Asp²-Glu³-Cys⁴-Glu⁵-Leu⁶-Cys⁷-Ile⁸-Asn⁹-Met¹⁰-Ala¹¹-Cys¹²-Thr¹³- | 167 |
| | | Gly¹⁴-Cys¹⁵-Ser¹⁶ | |
| N91 | C4:C12, C7:C15 | Gln¹-Glu²-Asp³-Cys⁴-Glu⁵-Leu⁶-Cys⁷-Ile⁸-Asn⁹-Met¹⁰-Ala¹¹-Cys¹²-Thr¹³- | 168 |
| | | Gly¹⁴-Cys¹⁵-Ser¹⁶ | |
| N92 | C4:C12, C7:C15 | Gln¹-Glu²-Glu³-Cys⁴-Glu⁵-Leu⁶-Cys⁷-Ile⁸-Asn⁹-Met¹⁰-Ala¹¹-Cys¹²-Thr¹³- | 169 |
| | | Gly¹⁴-Cys¹⁵-Ser¹⁶ | |
| N93 | C4:C12, C7:C15 | Lys¹-Asp²-Asp³-Cys⁴-Glu⁵-Leu⁶-Cys⁷-Ile⁸-Asn⁹-Met¹⁰-Ala¹¹-Cys¹²-Thr¹³- | 170 |
| | | Gly¹⁴-Cys¹⁵-Ser¹⁶ | |
| N94 | C4:C12, C7:C15 | Lys¹-Asp²-Glu³-Cys⁴-Glu⁵-Leu⁶-Cys⁷-Ile⁸-Asn⁹-Met¹⁰-Ala¹¹-Cys¹²-Thr¹³- | 171 |
| | | Gly¹⁴-Cys¹⁵-Ser¹⁶ | |
| N95 | C4:C12, C7:C15 | Lys¹-Glu²-Asp³-Cys⁴-Glu⁵-Leu⁶-Cys⁷-Ile⁸-Asn⁹-Met¹⁰-Ala¹¹-Cys¹²-Thr¹³- | 172 |
| | | Gly¹⁴-Cys¹⁵-Ser¹⁶ | |
| N96 | C4:C12, C7:C15 | Lys¹-Glu²-Glu³-Cys⁴-Glu⁵-Leu⁶-Cys⁷-Ile⁸-Asn⁹-Met¹⁰-Ala¹¹-Cys¹²-Thr¹³- | 173 |
| | | Gly¹⁴-Cys¹⁵-Ser¹⁶ | |

TABLE 5
Guanylin and Analogs

| | Position of | | SEQ |
|---|---|---|---|
| | Disulfide | | ID |
| Name | bonds | Structure | NO |
| Formula | 4:12, 7:15 | Xaa¹-Xaa²-Xaa³-Maa⁴-Xaa⁵-Xaa⁶-Maa⁷-Xaa⁸-Xaa⁹-Xaa¹⁰-Xaa¹¹-Maa¹²-Xaa¹³-Xaa¹⁴- | 174 |
| XIX | | Maa¹⁵ | |
| Guanylin | C4:C12, C7:C15 | Ser¹-His²-Thr³-Cys⁴-Glu⁵-Ile⁶-Cys⁷-Ala⁸-Phe⁹-Ala¹⁰-Ala¹¹-Cys¹²-Ala¹³-Gly¹⁴- | 175 |
| | | Cys¹⁵ | |
| Human | C4:C12, C7:C15 | Pro¹-Gly²-Thr³-Cys⁴-Glu⁵-Ile⁶-Cys⁷-Ala⁸-Tyr⁹-Ala¹⁰-Ala¹¹-Cys¹²-Thr¹³-Gly¹⁴- | 252 |
| Guanylin | | Cys¹⁵ | |
| N97 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴- | 176 |
| | | Cys¹⁵ | |
| N98 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴- | 177 |
| | | Cys¹⁵ | |
| N99 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴- | 178 |
| | | Cys¹⁵ | |
| N100 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴- | 179 |
| | | Cys¹⁵ | |
| N101 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴- | 180 |
| | | Cys¹⁵ | |
| N102 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴- | 181 |
| | | Cys¹⁵ | |
| N103 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴- | 182 |
| | | Cys¹⁵ | |
| N104 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴- | 183 |
| | | Cys¹⁵ | |
| N105 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴- | 184 |
| | | Cys¹⁵ | |
| N106 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴- | 185 |
| | | Cys¹⁵ | |
| N107 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴- | 186 |
| | | Cys¹⁵ | |
| N108 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴- | 187 |
| | | Cys¹⁵ | |
| N109 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴- | 188 |
| | | Cys¹⁵ | |
| N110 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴- | 189 |
| | | Cys¹⁵ | |
| N111 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴- | 190 |
| | | Cys¹⁵ | |
| N112 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴- | 191 |
| | | Cys¹⁵ | |
| N113 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴- | 192 |
| | | Cys¹⁵ | |
| N114 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴- | 193 |
| | | Cys¹⁵ | |
| N115 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴- | 194 |
| | | Cys¹⁵ | |
| N116 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴- | 195 |
| | | Cys¹⁵ | |
| N117 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴- | 196 |
| | | Cys¹⁵ | |
| N118 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴- | 197 |
| | | Cys¹⁵ | |
| N119 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴- | 198 |
| | | Cys¹⁵ | |
| N120 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴- | 199 |
| | | Cys¹⁵ | |
| N121 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴- | 200 |
| | | Cys¹⁵ | |
| N122 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴- | 201 |
| | | Cys¹⁵ | |
| N123 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴- | 202 |
| | | Cys¹⁵ | |
| N124 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴- | 203 |
| | | Cys¹⁵ | |
| N125 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴- | 204 |
| | | Cys¹⁵ | |
| N126 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴- | 205 |
| | | Cys¹⁵ | |
| N127 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴- | 206 |
| | | Cys¹⁵ | |
| N128 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴- | 207 |
| | | Cys¹⁵ | |

TABLE 6
Lymphoguanylin and Analogs

| Name | Position of Disulfide bonds | Structure | SEQ ID NO |
|---|---|---|---|
| FormulaXX | 4:12 | Xaa¹-Xaa²-Xaa³-Maa⁴-Xaa⁵-Xaa⁶-Maa⁷-Xaa⁸-Xaa⁹-Xaa¹⁰-Xaa¹¹-Maa¹²-Xaa¹³-Xaa¹⁴-Xaaₙ₁¹⁵ | 208 |
| Lymphoguanylin | C4:C12 | Gln¹-Glu²-Glu³-Cys⁴-Glu⁵-Leu⁶-Cys⁷-Ile⁸-Asn⁹-Met¹⁰-Ala¹¹-Cys¹²-Thr¹³-Gly¹⁴-Tyr¹⁵ | 209 |
| N129 | C4:C12 | Gln¹-Glu²-Glu³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 210 |
| N130 | C4:C12 | Gln¹-Asp²-Glu³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 211 |
| N131 | C4:C12 | Gln¹-Asp²-Asp³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 212 |
| N132 | C4:C12 | Gln¹-Glu²-Asp³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 213 |
| N133 | C4:C12 | Gln¹-Glu²-Glu³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 214 |
| N134 | C4:C12 | Gln¹-Asp²-Glu³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 215 |
| N135 | C4:C12 | Gln¹-Asp²-Asp³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 216 |
| N136 | C4:C12 | Gln¹-Glu²-Asp³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 217 |
| N137 | C4:C12 | Gln¹-Glu²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 218 |
| N138 | C4:C12 | Gln¹-Asp²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 219 |
| N139 | C4:C12 | Gln¹-Asp²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 220 |
| N140 | C4:C12 | Gln¹-Glu²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 221 |
| N141 | C4:C12 | Gln¹-Glu²-Glu³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 222 |
| N142 | C4:C12 | Gln¹-Asp²-Glu³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 223 |
| N143 | C4:C12 | Gln¹-Asp²-Asp³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 224 |
| N144 | C4:C12 | Gln¹-Glu²-Asp³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 225 |
| N145 | C4:C12, C7:C15 | Gln¹-Glu²-Glu³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵-Ser¹⁶ | 226 |
| N146 | C4:C12, C7:C15 | Gln¹-Asp²-Glu³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵-Ser¹⁶ | 227 |
| N147 | C4:C12, C7:C15 | Gln¹-Asp²-Asp³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵-Ser¹⁶ | 228 |
| N148 | C4:C12, C7:C15 | Gln¹-Glu²-Asp³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵-Ser¹⁶ | 229 |
| N149 | C4:C12, C7:C15 | Gln¹-Glu²-Glu³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵-Ser¹⁶ | 230 |
| N150 | C4:C12, C7:C15 | Gln¹-Asp²-Glu³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵-Ser | 231 |
| N151 | C4:C12, C7:C15 | Gln¹-Asp²-Asp³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵-Ser¹⁶ | 232 |
| N152 | C4:C12, C7:C15 | Gln¹-Glu²-Asp³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵-Ser¹⁶ | 233 |
| N153 | C4:C12, C7:C15 | Gln¹-Glu²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵-Ser¹⁶ | 234 |
| N154 | C4:C12, C7:C15 | Gln¹-Asp²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵-Ser¹⁶ | 235 |
| N155 | C4:C12, C7:C15 | Gln¹-Asp²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵-Ser¹⁶ | 236 |
| N156 | C4:C12, C7:C15 | Gln¹-Glu²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵-Ser¹⁶ | 237 |
| N157 | C4:C12, C7:C15 | Gln¹-Glu²-Glu³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵-Ser¹⁶ | 238 |
| N158 | C4:C12, C7:C15 | Gln¹-Asp²-Glu³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵-Ser¹⁶ | 239 |
| N159 | C4:C12, C7:C15 | Gln¹-Asp²-Asp³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵-Ser¹⁶ | 240 |
| N160 | C4:C12, C7:C15 | Gln¹-Glu²-Asp³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵-Ser¹⁶ | 241 |

TABLE 7
ST Peptide and Analogues

| | Position of Disulfide | | SEQ ID |
|---|---|---|---|
| Name | bonds | Structure | NO |
| STPeptide | C9:C14, C10:C18, C13:C21 | Asn¹-Ser²-Ser³-Asn⁴-Ser⁵-Ser⁶-Asn⁷-Tyr⁸-Cys⁹-Cys¹⁰- | 242 |
| | | Glu¹¹-Lys¹²-Cys¹³-Cys¹⁴-Asn¹⁵-Pro¹⁶-Ala¹⁷-Cys¹⁸-Thr¹⁹-Gly²⁰- | |
| | | Cys²¹-Tyr²² | |
| N161 | C3:C8, C4:C12, C7:C15 | PEG3-Asn¹-Phe²-Cys³-Cys⁴-Glu⁵-Thr⁶-Cys⁷-Cys⁸-Asn⁹-Pro¹⁰- | 243 |
| | | Ala¹¹-Cys¹²-Thr¹³-Gly¹⁴-Cys¹⁵-Tyr¹⁶-PEG3 | |
| N162 | C3:C8, C4:C12, C7:C15 | PEG3-Asn¹-Phe²-Cys³-Cys⁴-Glu⁵-Thr⁶-Cys⁷-Cys⁸-Asn⁹-Pro¹⁰- | 244 |
| | | Ala¹¹-Cys¹²-Thr¹³-Gly¹⁴-Cys¹⁵-Tyr¹⁶ | |
| N163 | C3:C8, C4:C12, C7:C15 | Asn¹-Phe²-Cys³-Cys⁴-Glu⁵-Thr⁶-Cys⁷-Cys⁸-Asn⁹-Pro¹⁰- | 245 |
| | | Ala¹¹-Cys¹²-Thr¹³-Gly¹⁴-Cys¹⁵-Tyr¹⁶-PEG3 | |
| N164 | C3:C8, C4:C12, C7:C15 | Asn¹-Phe²-Cys³-Cys⁴-Glu⁵-Tyr⁶-Cys⁷-Cys⁸-Asn⁹-Pro¹⁰- | 246 |
| | | Ala¹¹-Cys¹²-Thr¹³-Gly¹⁴-Cys¹⁵-Tyr¹⁶ | |
| N165 | C3:C8, C4:C12, C7:C15 | dAsn¹-Phe²-Cys³-Cys⁴-Glu⁵-Tyr⁶-Cys⁷-Cys⁸-Asn⁹-Pro¹⁰- | 247 |
| | | Ala¹¹-Cys¹²-Thr¹³-Gly¹⁴-Cys¹⁵-dTyr¹⁶ | |
| N166 | C3:C8, C4:C12, C7:C15 | Asn¹-Phe²-Cys³-Cys⁴-Glu⁵-Tyr⁶-Cys⁷-Cys⁸-Asn⁹-Pro¹⁰- | 248 |
| | | Ala¹¹-Cys¹²-Thr¹³-Gly¹⁴-Cys¹⁵-dTyr¹⁶ | |
| N167 | C3:C8, C4:C12, C7:C15 | dAsn¹-Phe²-Cys³-Cys⁴-Glu⁵-Tyr⁶-Cys⁷-Cys⁸-Asn⁹-Pro¹⁰- | 249 |
| | | Ala¹¹-Cys¹²-Thr¹³-Gly¹⁴-Cys¹⁵-Tyr¹⁶ | |

TABLE 8
Alpha-aminoadipic acid derivatives of GCRA Peptides

| Corresponds | Position of Disulfide | | |
|---|---|---|---|
| to: | bond | Structure | SEQ ID NO |
| SP-304 | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 259 |
| SP-326 | C3:C11, C6:C14 | Asp¹-Aad²-Cys³-Glu⁴-Leu⁵-Cys⁶-Val⁷-Asn⁸-Val⁹-Ala¹⁰-Cys¹¹-Thr¹²-Gly¹³-Cys¹⁴-Leu¹⁵ | 260 |
| SP-327 | C3:C11, C6:C14 | Asp¹-Aad²-Cys³-Glu⁴-Leu⁵-Cys⁶-Val⁷-Asn⁸-Val⁹-Ala¹⁰-Cys¹¹-Thr¹²-Gly¹³-Cys¹⁴ | 261 |
| SP-328 | C2:C10, C5:C13 | Aad¹-Cys²-Glu³-Leu⁴-Cys⁵-Val⁶-Asn⁷-Val⁸-Ala⁹-Cys¹⁰-Thr¹¹-Gly¹²-Cys¹³-Leu¹⁴ | 262 |
| SP-329 | C2:C10, C5:C13 | Aad¹-Cys²-Glu³-Leu⁴-Cys⁵-Val⁶-Asn⁷-Val⁸-Ala⁹-Cys¹⁰-Thr¹¹-Gly¹²-Cys¹³ | 263 |
| SP332 | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 264 |
| SP-333 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 253 |
| SP-334 | C4:C12, C7:C15 | dAsn¹-dAsp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 265 |
| SP-336 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 266 |
| SP-337 | C4:C12, C7:C15 | dAsn¹-Asp²-Aad³-Cys⁴-Glu⁵-dLeu⁶-Cys⁷-Val⁸-Asn⁹-Val¹⁰-Ala¹¹-Cys¹²-Thr¹³-Gly¹⁴-Cys¹⁵-dLeu¹⁶ | 267 |
| SP-338 | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵ | 268 |
| SP-342 | C4:C12, C7:C15 | PEG3-Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 269 |
| SP-343 | C4:C12, C7:C15 | PEG3-dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 270 |
| SP-344 | C4:C12, C7:C15 | PEG3-dAsn¹-dAsp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵⁻dLeu¹⁶-PEG3 | 271 |
| SP-347 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 272 |
| SP-348 | C4:C12, C7:C15 | PEG3-Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 273 |
| SP-350 | C4:C12, C7:C15 | PEG3-dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 274 |
| SP-352 | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 275 |
| SP-359 | C4:C12, C7:C15 | PEG3-dAsn¹-dAsp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 276 |
| SP-360 | C4:C12, C7:C15 | dAsn¹-dAsp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 277 |
| SP-368 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dNal¹⁶ | 280 |
| SP-369 | C4:C12, C7:C15 | dAsn¹-Asp²-Aad³-Cys⁴-Glu⁵-Leu⁶-Cys⁷-AIB⁸-Asn⁹-AIB¹⁰-Ala¹¹-Cys¹²-Thr¹³-Gly¹⁴-Cys¹⁵-dLeu¹⁶ | 281 |
| SP-370 | C4:C12, 7:15 | dAsn¹-Asp²-Aad³-Cys⁴-Glu⁵-Leu⁶-Asp[Lactam]⁷-Val⁸-Asn⁹-Val¹⁰-Ala¹¹-Cys¹²-Thr¹³-Gly¹⁴-Orn¹⁵-dLeu¹⁶ | 282 |
| SP-371 | C4:C12, C7:C15 | dAsn¹-Asp²-Aad³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 283 |
| SP-372 | C4:C12, C7:C15 | dAsn¹-Asp²-Aad³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 284 |
| N1 | C4:C12, C7:C15 | PEG3-dAsn¹-Asp²-Aad³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 285 |
| N2 | C4:C12, C7:C15 | PEG3-dAsn¹-Asp²-Aad³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 286 |
| N3 | C4:C12, C7:C15 | dAsn¹-Asp²-Aad³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 287 |
| N4 | C4:C12, C7:C15 | PEG3-dAsn¹-Asp²-Aad³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 288 |
| N5 | C4:C12, C7:C15 | PEG3-dAsn¹-Asp²-Aad³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 289 |
| N6 | C4:C12, C7:C15 | dAsn¹-Asp²-Aad³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 290 |
| N7 | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 291 |
| N8 | C4:C12, C7:C15 | PEG3-Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 292 |
| N9 | C4:C12, C7:C15 | PEG3-Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 293 |
| N10 | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 294 |
| N11 | C4:C12, C7:C15 | PEG3-Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 295 |
| N12 | C4:C12, C7:C15 | PEG3-Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶ | 296 |
| N13 | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 297 |
| Formula I | C4:C12, C7:C15 | Asn¹-Asp²-Aad³-Cys⁴-Xaa⁵-Xaa⁶-Cys⁷-Xaa⁸-Xaa⁹-Xaa¹⁰-Xaa¹¹-Cys¹²-Xaa¹³-Xaa¹⁴-Cys¹⁵-Xaa¹⁶ | 298 |
| (I-Aad) | | | |
| Formula II | C4:C12, C7:C15 | Xaaₙ₁-Cys⁴-Xaa⁵-Xaa⁶-Cys⁷-Xaa⁸-Xaa⁹-Xaa¹⁰-Xaa¹¹-Cys¹²-Xaa¹³-Xaa¹⁴-Cys¹⁵-Xaaₙ₂¹⁶ | 299 |
| (II-Aad) | | | |
| Formula | 4:12, 7:15 | Xaaₙ₁-Maa⁴-Glu⁵-Xaa⁶-Maa⁷-Val⁸-Asn⁹-Val¹⁰-Ala¹¹-Maa¹²-Thr¹³-Gly¹⁴-Maa¹⁵-Xaaₙ₂ | 300 |
| III | | | |
| (III-Aad) | | | |
| Formula | 4:12, 7:15 | Xaaₙ₁-Maa⁴-Xaa⁵-Xaa⁶-Maa⁷-Xaa⁸-Xaa⁹-Xaa¹⁰-Xaa¹¹-Maa¹²-Xaa¹³-Xaa¹⁴-Maa¹⁵-Xaaₙ₂ | 301 |
| IV | | | |
| (IV-Aad) | | | |
| Formula V | C4:C12, C7:C15 | Asn¹-Asp²-Aad³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-Xaa¹⁶ | 302 |
| (V-Aad) | | | |
| Formula | C4:C12, C7:C15 | dAsn¹-Glu²-Aad³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 303 |
| VI | | | |
| (VI-Aad) | | | |
| Formula | C4:C12, C7:C15 | dAsn¹-dGlu²-Aad³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 304 |
| VII-a | | | |
| (VI-a- | | | |
| Aad) | | | |
| Formula | C4:C12, C7:C15 | dAsn¹-dAsp²-Aad³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 305 |
| VII-b | | | |
| (VI-b- | | | |
| Aad) | | | |
| Formula | C4:C12, C7:C15 | dAsn¹-dAsp²-Aad³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 306 |
| VIII | | | |
| (VIII-Aad) | | | |
| Formula | C4:C12, C7:C15 | dAsn¹-dGlu²-Aad³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 307 |
| IX | | | |
| (IX-Aad) | | | |
| Formula | C4:C12, C7:C15 | Xaaₙ₁-Cys⁴-Xaa⁵-Xaa⁶-Xaa⁷-Xaa⁸-Xaa⁹-Xaa¹⁰-Xaa¹¹-Cys¹²-Xaa¹³-Xaa¹⁴-Xaa¹⁵-Xaaₙ₂¹⁶ | 257 |
| XXI | | | |
| (XXI- | | | |
| Aad) | | | |
| SP-363 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu-AMIDE¹⁶ | 308 |
| SP-364 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶ | 255 |
| SP-365 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer-AMIDE¹⁶ | 309 |
| SP-366 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dTyr¹⁶ | 256 |
| SP-367 | C4:C12, C7:C15 | dAsn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dTyr-AMIDE¹⁶ | 310 |
| SP-373 | C4:C12, C7:C15 | Pyglu¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu-AMIDE¹⁶ | 254 |
| / | C4:C12, C7:C15 | Pyglu¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 311 |
| SP- | C4:C12, C7:C15 | PEG3-Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶-PEG3 | 312 |
| 304diPEG | | | |
| SP-304N- | C4:C12, C7:C15 | PEG3-Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 313 |
| PEG | | | |
| SP-304C- | C4:C12, C7:C15 | Asn¹-Asp²-Glu³-Cys⁴-Aad⁵-Leu⁶-Cys⁷-Val⁸-Asn⁹-Val¹⁰-Ala¹¹-Cys¹²-Thr¹³-Gly¹⁴-Cys¹⁵-Leu¹⁶-PEG3 | 314 |
| PEG | | | |
| Formula | C4:C12, C7:C15 | Xaa¹-Xaa²-Aad³-Maa⁴-Xaa⁵-Xaa⁶-Maa⁷-Xaa⁸-Xaa⁹-Xaa¹⁰-Xaa¹¹-Maa¹²-Xaa¹³-Xaa¹⁴-Maa¹⁵-Xaa¹⁶ | 315 |
| XVIII | | | |
| (XVIII- | | | |
| Aad) | | | |
| N32 | C4:C12, C7:C15 | Glu¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 316 |
| N34 | C4:C12, C7:C15 | Glu¹-Glu²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 317 |
| N36 | C4:C12, C7:C15 | Asp¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 318 |
| N38 | C4:C12, C7:C15 | Asp¹-Glu²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 319 |
| N40 | C4:C12, C7:C15 | Gln¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 320 |
| N42 | C4:C12, C7:C15 | Gln¹-Glu²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 321 |
| N44 | C4:C12, C7:C15 | Lys¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 322 |
| N46 | C4:C12, C7:C15 | Lys¹-Glu²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 323 |
| N48 | C4:C12, C7:C15 | Glu¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 324 |
| N50 | C4:C12, C7:C15 | Glu¹-Glu²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 325 |
| N52 | C4:C12, C7:C15 | Asp¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 326 |
| N54 | C4:C12, C7:C15 | Asp¹-Glu²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 327 |
| N56 | C4:C12, C7:C15 | Gln¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 328 |
| N58 | C4:C12, C7:C15 | Gln¹-Glu²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 329 |
| N60 | C4:C12, C7:C15 | Lys¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 330 |
| N62 | C4:C12, C7:C15 | Lys¹-Glu²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 331 |
| N65 | C4:C12, C7:C15 | Glu¹-Asp²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Leu¹⁶ | 332 |
| N67 | C4:C12, C7:C15 | Glu¹-Glu²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Leu¹⁶ | 333 |
| N69 | C4:C12, C7:C15 | Asp¹-Asp²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Leu¹⁶ | 334 |
| N71 | C4:C12, C7:C15 | Asp¹-Glu²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Leu¹⁶ | 335 |
| N73 | C4:C12, C7:C15 | Gln¹-Asp²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Leu¹⁶ | 336 |
| N75 | C4:C12, C7:C15 | Gln¹-Glu²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Leu¹⁶ | 337 |
| N77 | C4:C12, C7:C15 | Lys¹-Asp²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Leu¹⁶ | 338 |
| N79 | C4:C12, C7:C15 | Lys¹-Glu²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Leu¹⁶ | 339 |
| N81 | C4:C12, C7:C15 | Glu¹-Asp²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 340 |
| N83 | C4:C12, C7:C15 | Glu¹-Glu²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 341 |
| N85 | C4:C12, C7:C15 | Asp¹-Asp²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 342 |
| N87 | C4:C12, C7:C15 | Asp¹-Glu²-Asp³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 343 |
| N88 | C4:C12, C7:C15 | Asp¹-Glu²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 344 |
| N89 | C4:C12, C7:C15 | Gln¹-Asp²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 345 |
| N91 | C4:C12, C7:C15 | Gln¹-Glu²-Asp³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 346 |
| N92 | C4:C12, C7:C15 | Gln¹-Glu²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 347 |
| N93 | C4:C12, C7:C15 | Lys¹-Asp²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 348 |
| N95 | C4:C12, C7:C15 | Lys¹-Glu²-Aad³-Cys⁴-Glu⁵-Leu⁶-Cys⁷-Ile⁸-Asn⁹-Met¹⁰-Ala¹¹-Cys¹²-Thr¹³-Gly¹⁴-Cys¹⁵-Ser¹⁶ | 349 |
| | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Thr¹⁶ | 350 |
| | C4:C12, C7:C15 | Asn¹-Asp²-Aad³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Tyr¹⁶ | 351 |

Linker

As used herein, a “linker” or “spacer” is any chemical moiety that is capable of linking the GCRA peptide to another moeity such as a component according to the invention. In some embodiments, a linker is attached to the N-terminus of the GCRA peptide. In some embodiments, a linker is attached to the C-terminus of the GCRA peptide. In some embodiments, a linker is attached to both the N- and C-termini. The linker may be any appropriate size. In some embodiments, the linker size affects GCRA peptide activity. In some embodiments, the linker is ten carbons or less long and the GCRA peptide retains activity. In some embodiments, the linker is ten carbons, nine carbons, eight carbons, seven carbons, six carbons, five carbons, four carbons, three carbons, two carbons, or one carbon.

Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or noncleavable linker). In some aspects, the linker is a procharged linker, a hydrophilic linker, or a dicarboxylic acid based linker.

In one aspect, the linker used is derived from a crosslinking reagent such as N-succinimidyl-3-(2-pyridyldithio)propionate (SPDP), N-succinimidyl 4-(2-pyridyldithio)pentanoate (SPP), N-succinimidyl 4-(2-pyridyldithio)butanoate (SPDB), N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-te-traazaheptadecan-1-oate (CX1-1). In another aspect, the linker used is derived from a cross-linking agent such as N-succinimidyl-3-(2-pyridyldithio)propionate (SPDP), N-succinimidyl 4-(maleimidomethyl) cyclohexanecarboxylate (SMCC), N-sulfosuccinimidyl 4-(maleimidomethyl) cyclohexanecarboxylate (sulfo-SMCC), N-succinimidyl-4-(2-pyridyldithio)-2-sulfo-butanoate (sulfo-SPDB) or 2,5-dioxopyrrolidin-1-yl 17-(2,5-dioxo-2,5-dihydro-1H-pyrrol-1-yl)-5,8,11,14-tetraoxo-4,7,10,13-te-traazaheptadecan-1-oate (CX1-1).

Non-cleavable linkers are any chemical moiety capable of linking a drug, such as a maytansinoid, to an antibody in a stable, covalent manner and does not fall off under the categories listed above for cleaveable linkers. Thus, non-cleavable linkers are substantially resistant to acid-induced cleavage, photo-induced cleavage, peptidase-induced cleavage, esterase-induced cleavage and disulfide bond cleavage. Furthermore, non-cleavable refers to the ability of the chemical bond in the linker or adjoining to the linker to withstand cleavage induced by an acid, photolabile-cleaving agent, a peptidase, an esterase, or a chemical or physiological compound that cleaves a disulfide bond, at conditions under which the drug, such as maytansionoid or the antibody does not lose its activity.

Acid-labile linkers are linkers cleavable at acidic pH. For example, certain intracellular compartments, such as endosomes and lysosomes, have an acidic pH (pH4-5), and provide conditions suitable to cleave acid-labile linkers.

Photo-labile linkers are linkers that are useful at the body surface and in many body cavities that are accessable to light. Furthermore, infrared light can penetrate tissue.

Some linkers can be cleaved by peptidases, i.e. peptidase cleavable linkers. Only certain peptides are readily cleaved inside or outside cells, see e.g. Trout et al., 79 Proc. Natl. Acad. Sci. USA, 626-629 (1982) and Umemoto et al. 43 Int. J. Cancer, 677-684 (1989). Furthermore, peptides are composed of .alpha.-amino acids and peptidic bonds, which chemically are amide bonds between the carboxylate of one amino acid and the amino group of a second amino acid. Other amide bonds, such as the bond between a carboxylate and the .epsilon.-amino group of lysine, are understood not to be peptidic bonds and are considered non-cleavable.

Some linkers can be cleaved by esterases, i.e. esterase cleavable linkers. Again, only certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

Procharged linkers are derived from charged cross-linking reagents that retain their charge after incorporation into an antibody drug conjugate. Examples of procharged linkers can be found in US 2009/0274713.

Components

The GCRA peptides may be linked to a component. The component is drug delivery vehicle, a chemotherapeutic agent, a micelle, a nanoparticle, a liposome, a polymer, a lipid, an imaging agent, and a labeling agent. In some embodiments, the component is biotin, and is followed by detection with avidin or streptavidin conjugated to fluorescence dye.

The chemotherapeutic agent is any compound known to treat cancer. For example, the chemotherapeutic agent can be methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH4556, CS-682, 9-AC, AG3340, AG3433, InceWX-710, VX-853, ZDO101, IS1641, ODN 698, TA 2516/Marmistat, BB2516/Marmistat, CDP 845, D2163, PD183805, DX8951f, Lemonal DP 2202, FK 317, Picibanil/OK-432, AD 32Nalrubicin, Metastron/strontium derivative, Temodal/Temozolomide, Evacet/liposomal doxorubicin, Yewtaxan/Placlitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/lrinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bisguanylhydrazone; MGBG), Pentostatin (2′deoxycoformycin), Semustine (methyl-CCNU), Teniposide (VM-26) and Vindesine sulfate. Furthermore, the chemotheraputic agent may be any of the chemotherapeutic agents mentioned in table 3 of U.S. Pat. No. 6,482,843 column 13 to 18.

Preferably, the chemotherapeutic agent is a taxane, an anthracyclin, a platin or 5-fluorouracil and any combination thereof.

An imaging agent or labeling agent is for example a magnetic tracer such as gadolinium chelate or a magnetic nanocrystal, such as for example a nanocrystal of iron, manganese oxide or iron-platinum (Fe—Pt); a radioactive tracer such as a radionuclide, such as for example ₁₂₃I. ₁₈F, ₁₁C, or a chelate of ₉₉mTc or ₁₁₁In, or a chelate of metal cations ₆₈Ga, ₆₄Cu or a flurophore

Preferably, the fluorophores used absorb and emit in the visible or near infrared range. The selected fluorophore is adapted to the type of application of the method: in-vivo or in-vitro. In the case of in-vivo applications, it is possible to resort to in-vivo fluorescence imaging. For this non-invasive imaging, the preferred fluorophores absorb and emit in the near infrared. Indeed, in order that the excitation light and the light emitted by the fluorophore may better cross the tissue, fluorophores absorbing and emitting in the near infrared should be used, i.e. at a wavelength comprised between 640 and 900 nm. During in-vitro applications, for which imaging methods are applied, such as fluorescence microscopy or cytometry of the FACS (Fluorescence Activating Cell Sorting) type, fluorophores are generally applied which are excited with visible wavelengths, typically comprised between 450 nm and 650 nm. These are notably hydrophilic cyanins or FITC (Fluorescein isothiocyanate). These fluorophores may be encapsulated in the lipid core of the nanoparticles or, when they are hydrophilic be localised at their surface.

As a lipophilic fluorophore, mention may for example be made of the compounds described in chapter 13 (“Probes for Lipids and Membranes”) of the InVitrogen catalogue (The Molecular Probes® Handbook, a guide to fluorescent probes and labeling technologies, 11.sup.th Edition, 2010). More specifically, mention may notably be made as a fluorophore, of indocyanine green (ICG), analogs of fatty acids, and the phospholipids functionalised with a fluorescent group such as the fluorescent products sold under the commercial names of Bodipy (R) such as Bodipy (R) 665/676 (Ex/Em.); lipophilic derivatives of carbocyanins such as 1.1′-dioctadecyl-3,3,3′,3′-tetramethylindodicarbocyanin perchlorate (DiD), for example sold under reference D-307, 3,3′-dihexadecyloxacarbocyanin perchlorate (DiO), for example sold under reference D1125, 1,1′-dihexadecyl-3,3,3′,3′-tetramethylindocarbocyanin perchlorate (Dil), for example sold under reference D384; the fluorescent probes derived from sphingolipids, from steroids or lipopolysaccharides such as the products sold under the commercial names BODIPY® TR ceramides, BODIPY® FL C5-lactosylceramide, BODIPY® FL C5-ganglioside, BODIPY® FL cerebrosides; amphiphilic derivatives of cyanins, rhodamines, fluoresceins or cumarins such as octadecyl rhodamine B, octadecyl fluorescein ester and 4-heptadecyl-7-hydroxycumarin; and diphenylhexatriene (DPH) and derivatives thereof; the whole of these products being sold by Invitrogen.

The drug delivery vehicles are for example liposomes, polymeric micelles, lipoprotein-based drug carriers, nanoparticle drug carriers, and dendrimers.

Disclosed nanoparticles may have a substantially spherical (i.e., the particles generally appear to be spherical), or non-spherical configuration. For instance, the particles, upon swelling or shrinkage, may adopt a non-spherical configuration. In some cases, the particles may include polymeric blends. For instance, a polymer blend may include a first co-polymer that includes polyethylene glycol and a second polymer.

In some embodiments, the nanoparticle is a PLA-based or PLGA-based nanoparticle. In some embodiments, the nanoparticle is an ACCURIN™ polymeric nanoparticle. In some embodiments, the conjugates disclosed herein maintain biological activity of a GCRA peptide. In some embodiments, the conjugated disclosed herein comprise a GCRA peptide conjugated to one or more components, such as a nanoparticle, and maintain biological activity of the GCRA peptide.

Disclosed nanoparticles may have a characteristic dimension of less than about 1 micrometer, where the characteristic dimension of a particle is the diameter of a perfect sphere having the same volume as the particle. For example, the particle can have a characteristic dimension of the particle can be less than about 300 nm, less than about 200 nm, less than about 150 nm, less than about 100 nm, less than about 50 nm, less than about 30 nm, less than about 10 nm, less than about 3 nm, or less than about 1 nm in some cases. In particular embodiments, disclosed nanoparticles may have a diameter of about 70 nm-200 nm, or about 70 nm to about 180 nm, about 80 nm to about 130 nm, about 80 nm to about 120 nm. In some embodiments, the particle has a characteristic dimension of the particle that is less than about 100 nm. In some embodiments, the therapeutic is formulated for intravenous administration and the nanoparticle has a characteristic dimension preferably less than 100 nm. In some embodiments, the therapeutic is formulated for oral administration (e.g. to the gastrointestinal tract) and the nanoparticle has a characteristic dimension up to 5 μm.

In some embodiments, the nanoparticle is not covered with long PEG, fatty acid, or other moieties (e.g. more than 30 side chains). In some embodiments, long PEG, fatty acid, or other moieties (e.g. more than 30 side chains) interfere with binding of the GCRA peptide to GC-C receptors expressed on a cell (e.g. metastatic cells, polyps, tumors, and other dysplastic lesions) and thus inhibit GCRA peptide activity.

In some embodiments, a nanoparticle is conjugated to the N-terminus of a GCRA peptide. In some embodiments, a nanoparticle is conjugated to the C-terminus of a GCRA peptide. In some embodiments, a nanoparticle is conjugated to both the N- and C-termini of a GCRA peptide. In some embodiments, a nanoparticle and another component are conjugated to a GCRA peptide. In some embodiments, a nanoparticle is conjugated at the C-terminus of a GCRA peptide and another component is conjugated at the N-terminus of a GCRA peptide. In some embodiments, a nanoparticle is conjugated at the N-terminus of a GCRA peptide and another component is conjugated at the C-terminus. In some embodiments, the other component is a chemotherapeutic. In one set of embodiments, the particles can have an interior and a surface, where the surface has a composition different from the interior, i.e., there may be at least one compound present in the interior but not present on the surface (or vice versa), and/or at least one compound is present in the interior and on the surface at differing concentrations. For example, in one embodiment, a compound, such as a targeting moiety (i.e., a low-molecular weight ligand) of a polymeric conjugate of the present invention, may be present in both the interior and the surface of the particle, but at a higher concentration on the surface than in the interior of the particle, although in some cases, the concentration in the interior of the particle may be essentially nonzero, i.e., there is a detectable amount of the compound present in the interior of the particle.

In some cases, the interior of the particle is more hydrophobic than the surface of the particle. For instance, the interior of the particle may be relatively hydrophobic with respect to the surface of the particle, and a drug or other payload may be hydrophobic, and readily associates with the relatively hydrophobic center of the particle. The drug or other payload can thus be contained within the interior of the particle, which can shelter it from the external environment surrounding the particle (or vice versa). For instance, a drug or other payload contained within a particle administered to a subject will be protected from a subject's body, and the body may also be substantially isolated from the drug for at least a period of time.

For example, disclosed herein is a therapeutic polymeric nanoparticle comprising a first non-functionalized polymer; an optional second non-functionalized polymer; an optional functionalized polymer comprising a targeting moiety; and a therapeutic agent. In a particular embodiment, the first non-functionalized polymer is PLA-based, PLGA-based, PEG-based, or copolymers thereof, e.g. a diblock co-polymer PLA-PEG. For example, exemplary nanoparticle may have a PEG corona with a density of about 0.065 g/cm.sup.3, or about 0.01 to about 0.10 g/cm³.

Disclosed nanoparticles may be stable (e.g. retain substantially all active agent) for example in a solution that may contain a saccharide, for at least about 3 days, about 4 days or at least about 5 days at room temperature, or at 25° C.

In some embodiments, disclosed nanoparticles may also include a fatty alcohol, which may increase the rate of drug release. For example, disclosed nanoparticles may include a C₈-C₃₀ alcohol such as cetyl alcohol, octanol, stearyl alcohol, arachidyl alcohol, docosonal, or octasonal.

Nanoparticles may have controlled release properties, e.g., may be capable of delivering an amount of active agent to a patient, e.g., to specific site in a patient, over an extended period of time, e.g. over 1 day, 1 week, or more. In some embodiments, disclosed nanoparticles substantially immediately releases (e.g. over about 1 minute to about 30 minutes) less than about 2%, less than about 4%, less than about 5%, or less than about 10% of an active agent (e.g. a taxane) agent, for example when places in a phosphate buffer solution at room temperature and/or at 37° C.

In one embodiment, the invention comprises a nanoparticle comprising 1) a polymeric matrix and 2) an amphiphilic compound or layer that surrounds or is dispersed within the polymeric matrix forming a continuous or discontinuous shell for the particle. An amphiphilic layer can reduce water penetration into the nanoparticle, thereby enhancing drug encapsulation efficiency and slowing drug release. Further, these amphipilic layer protected nanoparticles can provide therapeutic advantages by releasing the encapsulated drug and polymer at appropriate times.

As used herein, the term “amphiphilic” refers to a property where a molecule has both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. Exemplary amphiphilic compound include, for example, one or a plurality of the following: naturally derived lipids, surfactants, or synthesized compounds with both hydrophilic and hydrophobic moieties.

Specific examples of amphiphilic compounds include, but are not limited to, phospholipids, such as 1,2 distearyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and .beta.-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

In a particular embodiment, an amphiphilic component may include lecithin, and/or in particular, phosphatidylcholine.

Another aspect of the invention is directed to systems and methods of making disclosed nanoparticles. In some embodiments, using two or more different polymers (e.g., a copolymer such as a diblock copolymer and a homopolymer) properties of particles may be controlled.

In a particular embodiment, the methods described herein form nanoparticles that have a high amount of encapsulated therapeutic agent, for example, may include about 1 to about 40 weight percent, or about 1 to about 30 weight percent, e.g. about 10 to about 25 weight percent or about 5 to about 20 weight percent therapeutic agent.

In some embodiments, the methods described herein can be used to make nanoparticles containing up to 10 μM of a component (e.g. drug delivery vehicle, a chemotherapeutic agent, a micelle, a nanoparticle, a liposome, a polymer, a lipid, an imaging agent, or a labeling agent). In some embodiments, the methods described herein can be used to make nanoparticles containing from 100 nm to 10 μM of a component. In some embodiments, the component is a chemotherapeutic. In some embodiments, the methods described herein can be used to make nanoparticles containing up to 10 μM of a GCRA peptide disclosed herein. In some embodiments, the methods described herein can be used to make nanoparticles containing from 100 nm to 10 μM of a GCRA peptide disclosed herein.

In some embodiments, the methods described herein can be used to make nanoparticles containing varied ratios of component to GCRA peptide. In some embodiments, the ratio of component to GCRA peptide is 1:1, 2:1, 3:1, 4:1, 5:1, 6:1, 7:1, 8:1, 9:1, or 10:1. In some embodiments, the ratio of component to GCRA peptide is 1:2, 1:3, 1:4, 1:5, 1:6, 1:7, 1:8, 1:9, or 1:10. In some embodiments, the component is a chemotherapeutic and the ratio of chemotherapeutic to GCRA peptide is 10:1.

Liposomes and Controlled Release:

In order to design a drug delivery system, various kinds of high performance carrier materials are being developed to deliver the necessary amount of drug to the targeted site for a necessary period of time, both efficiently and precisely.

Cyclodextrins, biodegradable or non biodegradable polymers, liposomes, emulsions. Multiple emulsions are potential candidates for such a role, because of their ability to alter physical, chemical and biological properties of guest molecules. There are number of drug delivery systems including but not limited to polymer microcapsules, microparticles, nanoparticles, liposomes and emulsion. Many of these are prepared from synthetic biodegradable polymers such as polyanhydrides and poly hydroxy acids. In these systems the drugs incorporate in polymeric microspheres, which release the drug inside the organism in small and controlled daily doses during days months or until years.

Several polymers already were tested in controlled release systems. Such as: polyuretans for its elasticity, polysiloxans or silicons for being a good one insulating, polymethyl-metacrilate for its physical form; polyvinylalcohol for its hydrofobicity and resistance, polyethilene for its hardness and impermeability (Gilding, D. K. Biodegradable polymers. Biocompat. Clin. Impl. Mater. 2:209-232, 1981). Biodegradable polymers and biocompatible polymers, have been extensively investigated as vehicle for controlled release systems due to their ability to undergo surface degradation. These kind of polymers can be chose from: poly(2-hidroxi-ethylmetacrilate), polyacrilamide, polymer from lactic acid (PLA), from glicolic acid (PGA), and the respective ones co-polymers, (PLGA) and the poly(anidrides), as described by Tamada and Langer, J. Biomater. Sci. Polym. Edn, 3(4):315-353.

Suitable controlled release vehicles include, but are not limited to, biocompatible polymers, other polymeric matrices, capsules, microcapsules, nanocapsules, microparticles, nanoparticles, bolus preparations, osmotic pumps, diffusion devices, liposomes, lipospheres and transdermal delivery systems, implantable or not.

Satisfactory systems of controlled release include, but are not limited to, the ciclodextrines, biocompatible polymers, biodegradable polymers, other polymeric matrixes, capsules, micro-capsules, microparticles, bolus preparations, osmotic pumps, diffusion devices, lipossomes, lipoesferes, and systems of transdermic administration. Other compositions of controlled release include liquids that, when submitted the temperature changes, form a solid or a gel in situ.

Liposomes are lipid vesicles that include aqueous internal compartments in which molecules, for example drugs, are encapsulated with the objective of reaching a controlled release of the drug after administration in individuals. Many different techniques have been proposed for the preparation of liposomes [U.S. Pat. No. 4,552,803, Lenk; U.S. Pat. No. 4,310,506, Baldeschwieler; U.S. Pat. No. 4,235,871, Papahadjopoulos; U.S. Pat. No. 4,224,179, Schneider; U.S. Pat. No. 4,078,052, Papahadjopoulos; U.S. Pat. No. 4,394,372, Tailor; U.S. Pat. No. 4,308,166, Marchetti; U.S. Pat. No. 4,485,054, Mezei; and U.S. Pat. No. 4,508,703, Redziniak; Woodle and Papahadjopoulos, Methods Enzymol. 171:193-215 (1989]; Unilamellar vesicles display a single membrane [Huang, Biochemistry 8:334-352 (1969] while muitilamellar vesicles (MLVs) have numerous concentric membranes [Bangham et al., J. Mol. Biol. 13:238-252 (1965]. The procedure of Bangham [J. Mol. Biol. 13:238-252 (1965] produces “ordinary MLVs”, that present unequal solute distributions among the aqueous compartments and, consequently, differences of osmotic pressure. Lenk et al. (U.S. Pat. No. 4,522,803; U.S. Pat. No. 5,030,453 and U.S. Pat. No. 5,169,637), Fountain et al. (U.S. Pat. No. 4,588,578), Cullis et al. (U.S. Pat. No. 4,975,282) and Gregoriadis et al. (Pat. W.O. 99/65465) introduced methods for the preparation of MLVs that present substantially equal solute distributions among the compartments. Similar solute distributions among the different compartments mean a larger drug encapsulation efficiency as well as smaller differences of osmotic pressure that turns these MLVs more stable than ordinary MLVs. Unilamellar vesicles can be produced by sonication of MLVs [Papahadjopoulos et al. (1968)] or by extrusion through polycarbonate membranes [Cullis et al. (U.S. Pat. No. 5,008,050) and Loughrey et al. (U.S. Pat. No. 5,059,421)].

Satisfactory lipids include for example, phosphatidylcholine, phosphatidylserine, phosphatidylglycerol, cardiolipin, cholesterol, phosphatidic acid, sphingolipids, glycolipids, fatty acids, sterols, phosphatidylethanolamine, polymerizable lipids in their polymerized or non-polymerized form, mixture of these lipids.

The composition of the liposomes can be manipulated such as to turn them specific for an organ or a cell type. The targeting of liposomes has been classified either on the basis of anatomical factors or on the basis of the mechanism of their interaction with the environment. The anatomical classification is based on their level of selectivity, for example, organ-specific or cell-specific. From the point of view of the mechanisms, the--targeting can be considered as passive or active.

The passive targeting exploits the natural tendency of conventional liposomes to be captured by the cells of the reticulo-endotheliai system, i.e. mainly the fixed macrophages in the liver, spleen and bone marrow.

Sterically stabilized liposomes (also well-known as “PEG-liposomes”) are characterized by a reduced rate of elimination from the blood circulation [Lasic and Martin, Stealth Liposomes, CRC Press, Inc., Boca Raton, Fla. (1995)].

PEG-liposomes present a polyethylene glycol polymer conjugated to the head group of some phospholipid that reduces their interaction with plasma proteins, such as opsonins, and reduces the rate of their uptake by cells. The resulting steric barrier allows these liposomes to remain for a longer period of time within the circulation than conventional liposomes [Lasic and Martin, Stealth Liposomes, CRC Press, Inc., Boca Raton, Fla. (1995); Woodle et al., Biochim. Biophys. Acta 1105:193-200 (1992); Litzinger et al., Biochim. Biophys. Acta 1190:99-107 (1994); Bedu Addo, et al., Pharm. Res. 13:718-724 (1996]. The drug encapsulation within PEG-liposomes has resulted in the improvement of the effectiveness of many chemotherapeutic agents [Lasic and Martin, Stealth liposomes, CRC Press, Inc., Boca Raton, Fla. (1995)] and bioactive peptides [Allen T. M. In: Liposomes, New Systems, New Trends in their Applications (F. Puisieux, P. Couvreur, J. Delattre, J.-P. Devissaguet Ed.), Editions de la Sante, France, 1995, pp. 125].

Studies in this area demonstrated that different factors affect the effectiveness of PEG-liposomes. Ideally, the diameter of the vesicles should be below 200 nm, the number of units in PEG of approximately 2.000 and the proportion of Pegylated lipid from 3 to 5 mol % [Lasic and Martin, Stealth Liposomes, CRC Press, Inc., Boca Raton, Fla. (1995); Woodle et al., Biochim. Biophys. Acta 1105:193-200 (1992); Litzinger et al., Biochim. Biophys. Acta 1190:99-107(1994); Bedu Addo et al., Pharm. Res. 13:718-724(1996)].

The active targeting involves alteration of liposomes through their association with a liguand, such as a monoclonal antibody, a sugar, a glycolipid, protein, a polymer or by changing the lipid composition or the liposome size to target them to organs and cells different from those which accumulate conventional liposomes.

Peptide conjugated micelle (Layek et al. “Cell Penetrating Peptide Conjugated Polymeric Micelles as a High Performance Versatile Nonviral Gene Carrier” Biomacromolecules 2013, 14, 4071-4081).

Peptide-drug conjugates: peptide-drug-conjugates (Aran et al. “Cancer treatment by targeted drug delivery to tumor vasculature in a mouse model” Science 1998, 279(5349):377-80; Forner et al. “Peptide-drug conjugates: types, utility & Manufacturing” Specially Chemicals Magazine May 2012, p 46-47; Firer et al. “Targeted drug delivery for cancer therapy: the other side of antibodies” Journal of Hematology & Oncology 2012, 5:70; Majumdar et al. “Peptide-Mediated Targeted Drug Delivery” Medicinal Research Reviews 32, No. 3, 637-658, 2012; Zhang et al. “Cellular Uptake and Cytotoxicity of Drug-Peptide Conjugates Regulated by Conjugation Site” Bioconjugate Chemistry 2013, 24, 604-613; Lelle et a. “Novel cleavable cell-penetrating peptide-drug conjugates: synthesis and characterization” J. of Peptide science 2014; 20: 323-333).

Peptide-Polymer Conjugates: Annu Rev Phys Chem. 2013; 64:631-57; Cancer vaccines: (Arens et al. “Prospects of combinatorial synthetic peptide vaccine-based immunotherapy against cancer” Seminars in Immunology 25 (2013) 182-190).

Peptide conjugated to magnetic nanoparticle: (Xie et al. “Surface-engineered magnetic nanoparticle platforms for cancer imaging and therapy” Accounts of Chemical Research 44(10) 883-892, 2011).

Peptide-dendrimer conjugates: (Liu et al. “Novel peptide-dendrimer conjugates as drug carriers for targeting non-small cell lung cancer” International J. of Nanomedicine 2011:6 59-69).

Radiolabeled Peptides: (Fani et al. “Radiolabeled Peptides: Valuable Tools for the Detection and Treatment of Cancer” Theranostics 2012, 2(5): 481-501).

Peptide conjugates for cancer molecular imaging such as Fe.sub.3O.sub.4: Anticancer Agents Med Chem. 2012;

Treatment Methods

The GRCA peptide conjugates of the invention are generally useful as cancer therapeutics or prophylactics and are administered in vivo. They can be administered to mammalian subjects (e.g., human colon cancer patients) alone or in conjunction with other drugs and/or radiotherapy. The compounds can also be administered to subjects that are genetically and/or environmentally (due to, for example, physiological and/or environmental factors) susceptible to cancer, e.g., subjects with a family history of cancer (e.g., colon cancer), subjects with chronic inflammation or subject to chronic stress, or subjects that are exposed to natural or non-natural environmental carcinogenic conditions (e.g., excessive exposure to sunlight, industrial carcinogens, or tobacco smoke).

Cancer cells to which the methods of the present invention can be applied include generally any cell that expresses guanylate cyclase C receptors. An appropriate cell is for example colon cancer cell, a metastatic colon cancer cell, a colonic polyp, a colonic adenoma or a colonic microadenoma. For purposes of the present invention, the at risk population of one or more of the mammals to be treated includes those (1) having been diagnosed with colon cancer, colonic adenomas, and/or colonic microadenomas; and/or (2) having a close blood relative who has been diagnosed with colon cancer, colonic adenomas, and/or colonic microadenomas. In addition, the methods of the invention can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.0001 mg/kg-100 mg/kg. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 4-, 5-, 6-, 8-, 10-, 20-, 50-, 100-, 150-, or more times). Encapsulation of the polypeptide in a suitable delivery vehicle (e.g., polymeric microparticles or implantable devices) may increase the efficiency of delivery, particularly for oral delivery. In some embodiments, the route of administration depends on the use of the therapeutic or the type of cancer being detected/treated. For example, in some embodiments, the therapeutic is used for detection of polyps, tumors, and other dysplatic lesions in the GI tract and is formulated for oral administration. In some embodiments, the therapeutic is used for detection of colorectal metastases (e.g. in blood and other organs of the body) and is formulated for intravenous administration.

Therapeutic Administration and Formulations

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed., Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci.89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

In some embodiments, the conjugates according to the invention—referred to collectively herein as the Therapeutic(s)—are administered in conjunction with one or more additional agents, or a combination of additional agents. Suitable additional agents include current pharmaceutical and/or surgical therapies for an intended application. For example, the Therapeutic(s) can be used in conjunction with an additional chemotherapeutic or anti-neoplastic agent. For example, the Therapeutic(s) and additional agent are formulated into a single therapeutic composition, and the Therapeutic(s) and additional agent are administered simultaneously. In some embodiments, the Therapeutic(s) and additional agent are separate from each other, e.g., each is formulated into a separate therapeutic composition, and the Therapeutic(s) and the additional agent are administered simultaneously, or the Therapeutic(s) and the additional agent are administered at different times during a treatment regimen. For example, the Therapeutic(s) is administered prior to the administration of the additional agent, the Therapeutic(s) is administered subsequent to the administration of the additional agent, or the Therapeutic(s) and the additional agent are administered in an alternating fashion. As described herein, the Therapeutic(s) and additional agent are administered in single doses or in multiple doses.

In some embodiments, the additional agent is coupled or otherwise attached to the Therapeutic(s).

Suitable additional agents are selected according to the purpose of the intended application (i.e., killing, prevention of cell proliferation, hormone therapy or gene therapy). Such agents may include but is not limited to, for example, pharmaceutical agents, toxins, fragments of toxins, alkylating agents, enzymes, antibiotics, antimetabolites, antiproliferative agents, hormones, neurotransmitters, DNA, RNA, siRNA, oligonucleotides, antisense RNA, aptamers, diagnostics, radiopaque dyes, radioactive isotopes, fluorogenic compounds, magnetic labels, nanoparticles, marker compounds, lectins, compounds that alter cell membrane permeability, photochemical compounds, small molecules, liposomes, micelles, gene therapy vectors, viral vectors, and the like. Finally, combinations of agents or combinations of different classes of agents may be used.

The conjugates of the disclosure (also referred to herein as “Therapeutic(s)” or “active compounds”), and derivatives, fragments, analogs and homologs thereof, can be incorporated into pharmaceutical compositions suitable for administration. Principles and considerations involved in preparing such compositions, as well as guidance in the choice of components are provided, for example, in Remington's Pharmaceutical Sciences: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Such compositions typically comprise the conjugates of the disclosure and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated.

The formulations to be used for in vivo administration must be sterile. This is readily accomplished by filtration through sterile filtration membranes.

A pharmaceutical composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be suitable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as sustained/controlled release formulations, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

For example, the active ingredients can be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The materials can also be obtained commercially from Alza Corporation and Nova Pharmaceuticals, Inc. Liposomal suspensions (including liposomes targeted to infected cells with monoclonal antibodies to viral antigens) and can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The formulation can also contain more than one active compound as necessary for the particular indication being treated, for example, those with complementary activities that do not adversely affect each other. In some embodiments, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In one embodiment, the active compounds are administered in combination therapy, i.e., combined with other agents, e.g., therapeutic agents, that are useful for treating colonic adenomas, polyps, colon cancer and its metastases. The term “in combination” in this context means that the agents are given substantially contemporaneously, either simultaneously or sequentially. If given sequentially, at the onset of administration of the second compound, the first of the two compounds is still detectable at effective concentrations at the site of treatment.

Such combination therapies may advantageously utilize lower dosages of the administered therapeutic agents, thus avoiding possible toxicities or complications associated with the various monotherapies.

Definitions

As used herein. “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “drug delivery vehicles” refers to a vehicle that is capable of delivering medication to a patient in a manner that increases the concentration of the medication in some parts of the body relative to others. Drug delivery vehicles includes, but not limited to, polymeric micelles, liposomes, lipoprotein-based drug carriers, nano-particle drug carriers, dendrimers, cells, polypeptides, etc. An ideal drug delivery vehicle must be non-toxic, biocompatible, non-immunogenic, biodegradable, and must avoid recognition by the host's defense mechanisms. The term “treating” or “treatment” refers to administration of an effective amount of the compound to a subject in need thereof, who has cancer, or a symptom or predisposition toward such a disease, with the purpose of cure, alleviate, relieve, remedy, ameliorate, or prevent the disease, the symptoms of it, or the predisposition towards it. Such a subject can be identified by a health care professional based on results from any suitable diagnostic method.

“An effective amount” refers to the amount of an active compound that is required to confer a therapeutic effect on the treated subject. Effective doses will vary, as recognized by those skilled in the art, depending on rout of administration, excipient usage, and the possibility of co-usage with other therapeutic treatment.

The “Guidance for industry and Reviewers Estimating the Safe Starting Dose in Clinical Trials for Therapeutics in Adult Healthy Volunteers” published by the U.S. Department of Health and Human Services Food and Drug Administration discloses a “therapeutically effective amount” may be obtained by calculations from the following formula: HED=animal dose in mg/kg×(animal weight in kg/human weight in kg) 0.33

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., a fluorophore, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1: Synthesis of Sp-333-IR680 Probe

SP-333 was labeled with an appropriate infrared (IR) fluorescence dye for in vivo imaging purposes. The peptide was to be labeled with an appropriate IR dye through the reaction of a N-hydroxysuccinamidyl (NHS) activated ester with the amine group of the peptide. Our initial test with the VivoTag680 NHS ester supplied by PerkinElmer, Inc was not successful. Hence, we explored conjugation of fluorescence dye, Cy3-NHS ester and Cy5.5 NHS ester with SP-333. Labeling SP-333 with Cy3 and the subsequent HPLC purification was successful, with an overall labeling efficiency of 30% achieved. The spectroscopic properties for Cy5.5 were comparable with those of VivoTag680. Both Cy3 and Cy5.5 belong to a family of cyanine fluorescence dyes used in bioimaging applications. We performed a number of experiments and determined that these dyes could be conjugated to the N-terminal of SP-333 without appreciable loss of its binding to GC-C receptors. Although the conjugation and subsequent purification of either of the dyes to SP-333 was successful the generated Cy3-SP-333 and Cy5.5-SP-333 failed to exhibit specific binding to GC-C receptors expressed on the surface of T84 cells. We performed similar binding experiments with the cell membranes isolated from T84 cells and from rat intestinal tissues. Again, both of these probes did not show any specific binding. Similar results showing lack of specific binding were also observed in ex vivo binding with rat intestinal fragments. Thus, SP-333-IR680 fluorescence was custom synthesized.

Custom Synthesis Process Description

Buffer Preparation:

Sorenson's phosphate buffer (pH8.0-8.5) was prepared according to the ratio below:
Solution A: weigh Na2HPO4.12H2O (71.64g), dissolved in DI water and QS to 1000 mL with DI water;
Solution B: Weigh NaH2PO4.2H2O (31.21g), dissolved in DI water and QS to 1000 mL with DI water;
   Mix solution a (47.35 mL) with solution B (2.65 mL) thoroughly, dilute with DI water to 100 mL. The pH of the solution was 8.09 measured with a pH meter.

IPC HPLC Method

In Process Analytical HPLC Method Parameters

| | |
|---|---|
| Column | Kromasil, C18, 5 um, 100 Å |
| Column Dimensions | 250 × 4.6 mm |
| Column Temperature | 40° C. |
| Wavelength | 215 nm |
| Flow rate | 1.0 mL/minute |
| Buffer A | 0.1% TFA in water |
| Buffer B | 0.1% TFA in ACN |
| Gradient | Buffer B: → 5% 95% in 50 min |

Conjugation:

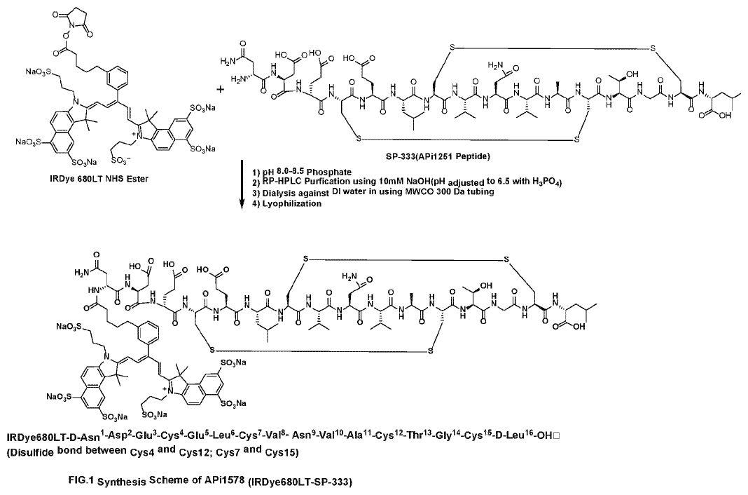

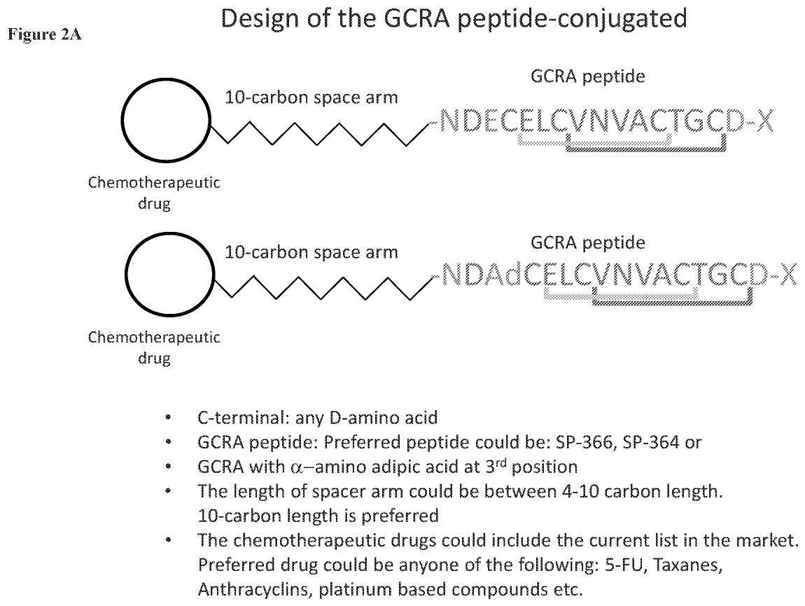

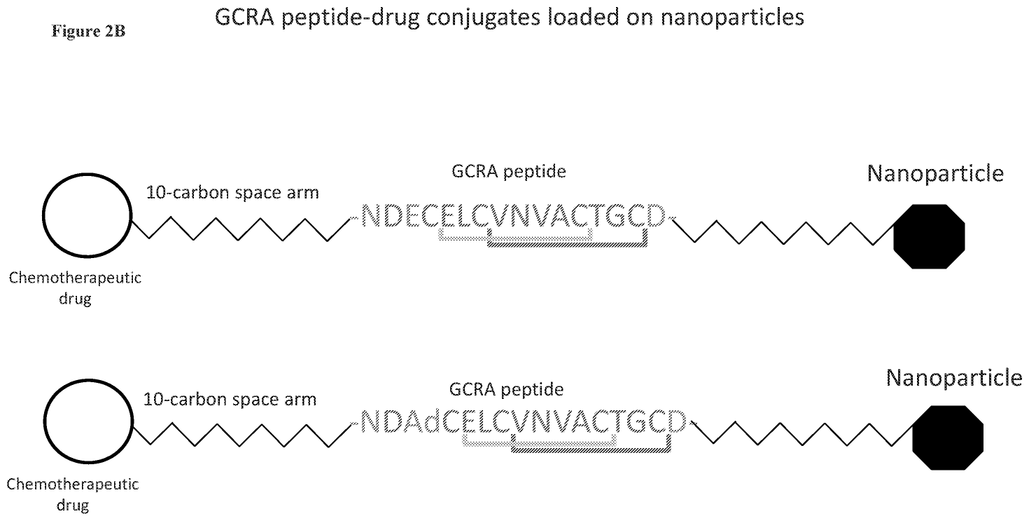

Api 1251 (SP-333) acetate (49.6 mg, 0.0285 mmol, 1.0 eq) was dissolved in 1.5 mL of Sorenson's phosphate buffer (pH8.09) above with sonification. To the above clear peptide solution was added IRDye-680LT NHS ester (40 mg, 0.0285 mol, 1.0 eq). The residual due material in the bottle was rinsed with 1.05 mL of Sorenson's phosphate buffer (pH8.09) and added to the reaction mixture. The reaction was continued at ambient temperature (20 to 28° C.) overnight, protected from light. The reaction was monitored with HPLC. After completion of the reaction, the reaction mixture was filtered and the filtrate was loaded onto the HPLC column directly. (FIG. 1)

RP-HPLC Purification:

The peptide solution was filtered through a 1.2 um membrane and then loaded to a5-cm diameter column packed with Kromasil 10 μm, C18 solid phase media to a bed height of 25 cm. and operated by a solvent delivery system. Preparative RP-HPLC purification was performed in a 10 mM NaOH in water, pH=6.5 (pH adjustation with H3PO4) buffer system with gradient elution from 11-35% organic constituent in 80 min. at a flow rate of 90 mL/min. The peptide of interest was collected in fractions, which were analyzed via HPLC to assess the overall purity.

Dialysis and Lyophilization:

The collected purification fractions were dialyzed (using a 300 MWCO membrane)in a 1 L container against deionized water for two days, replacing the water every 4-8 hours. The dialyzed peptide solution is then isolated by freeze-drying for 72 hrs to yield the final product of APi1578.

The approach produced a the bioactive IR680-SP-333 probe. All of the in vitro, ex vivo and in vivostudies described below were performed using this probe.

### Example 2: Specific Binding of IR680-SP-333 to GC-C on T84 Cells

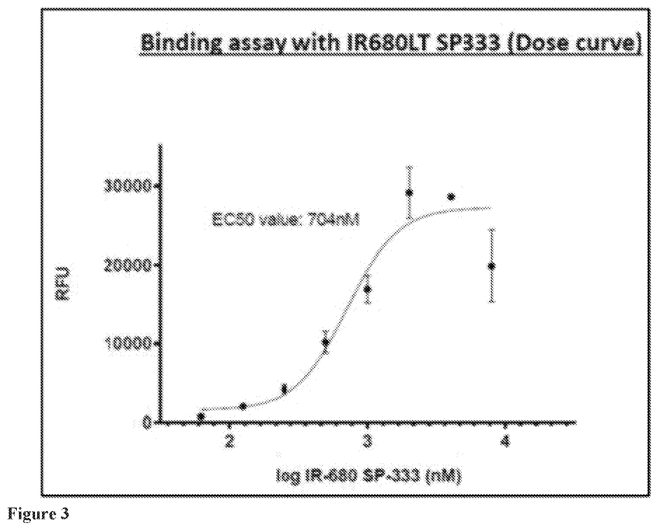

Experiments were performed to establish binding conditions to reduce background and to enhance specific binding. These experiments concluded that the age of T84 monolayer should be between 7-9 days after seeding to allow maximum expression of GC-C on the cell surface. Next, we established the optimal binding buffer, incubation time to allow binding of probe to T84 cells and temperature of binding incubation to minimize endocytosis. The optimal conditions for specific binding of probe to T84 cells are briefly described here. Human T84 cells were grown to confluence in 96 black clear bottom plates. Media was aspirated and the plate was blocked with 200 ul/well of blocking buffer (DMEM/F12+150 mM NaCl+20 mM Sodium phosphate dibasic, pH was adjusted to 6.0 with 1N HCl) for 2 hours in cell culture incubator at 37° C. Blocking buffer was aspirated and 50 ul/well of binding buffer (DMEM/F12+150 mM NaCl+20 mM Sodium phosphate dibasic, pH adjusted to 6.0 with 1N HCl, was added for 10 minutes to precondition the cells. This was followed by addition of 50 ul/well of IR680-SP333. Plate was incubated at 4° C. for 1 hour. After incubation, plate was washed with 200 ul/well of chilled binding buffer twice and 100 ul/well of chilled PBS, pH7.2 was added and plate was read at ex680, em715 using Tecan F200 and results were plotted using the iControl software. As shown in FIG. 3, the probe specifically bound to GC-C expressed on T84 cells in a dose-dependent manner. The binding of probe was considerably reduced in the presence of 100-fold excess of unlabeled SP-333, suggesting that the binding of the probe was specific to GC-C receptors.

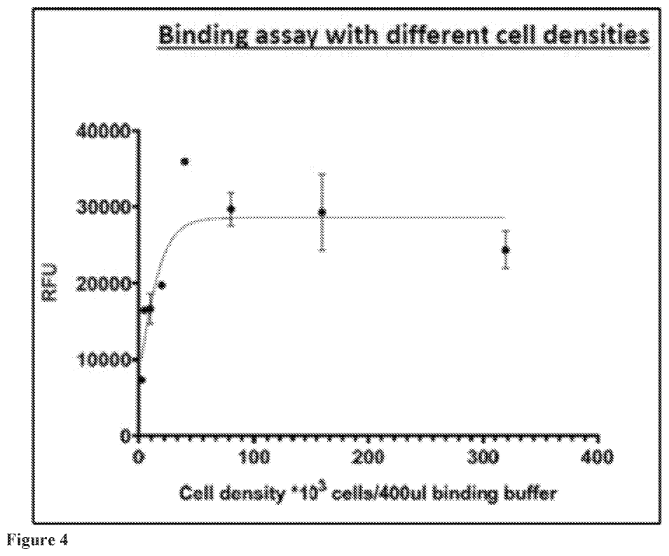

Next, we performed binding of probe to suspension of T84 cells. In this set of experiments, cells cultured for 9 days in T75 flasks were harvested by scraping and suspended in pre-chilled binding media. Different densities of cells were incubated with 200 mL IR680-SP-333 (final concentration 0.5 μM) in pre-chilled binding buffer at 4° C. for 30 mins. The IR680-SP-333 was diluted with unlabeled SP-333 to adjust the final concentration of probe 0.5 μM. This dilution was necessary to saturate the available GC-C binding sites on 40,000 cells. As shown in FIG. 4, the optimal cell density for binding was determined to be 40,000 cells for 0.5 μM of the probe. The binding of probe to cell suspension was abolished in the presence of 100-fold excess of SP-333, demonstrating the specificity of binding.

### Example 3: Demonstration of Binding Specificity of IR680-SP-333

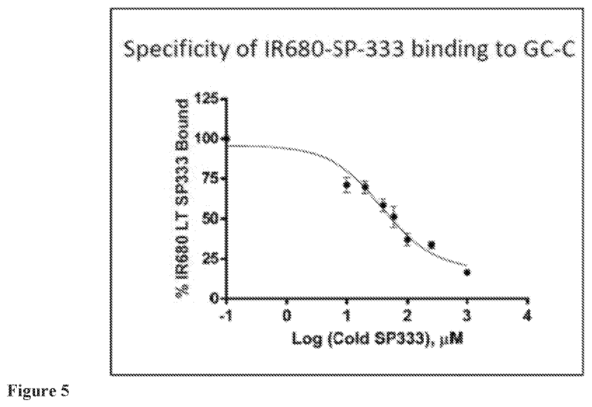

The specificity of IR680-SP-333 binding to T84 cells was further demonstrated by competition with increasing concentrations of unlabeled SP-333. As shown in FIG. 5, the binding of the probe was inhibited by unlabeled SP-333 in a dose dependent manner with IC₅₀ value of approximately 1.6 μM. This experiment was repeated several times with two different batches to confirm that the probe binds specifically to the GC-C on T84 cell surface. Further, as shown in FIG. 6, the potency of unconjugated SP-333 and SP-333 conjugated to IR680 or biotin in T84 cGMP bioassay. IR680 NHS ester (-5-6 carbon equivalent) is directly conjugated to SP-333 while biotin is conjugated to SP-333 via 2 amino ethoxy acetic acid (AEEA; each ˜9-10 carbon chain equivalent). Both conjugates are active as evidenced by their ability to stimulate cGMP production in T84 cells although at a slightly lower level as compared to unconjugated SP-333.

### Example 4: Ex-Vivo and In-Vivo Detection of Colonc Adenomas

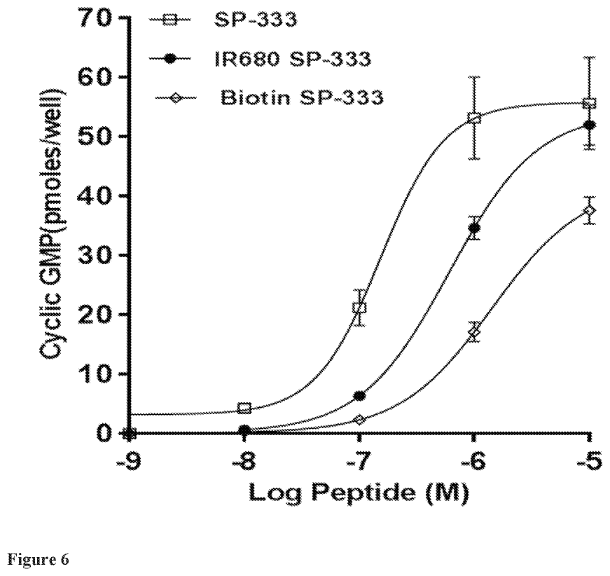

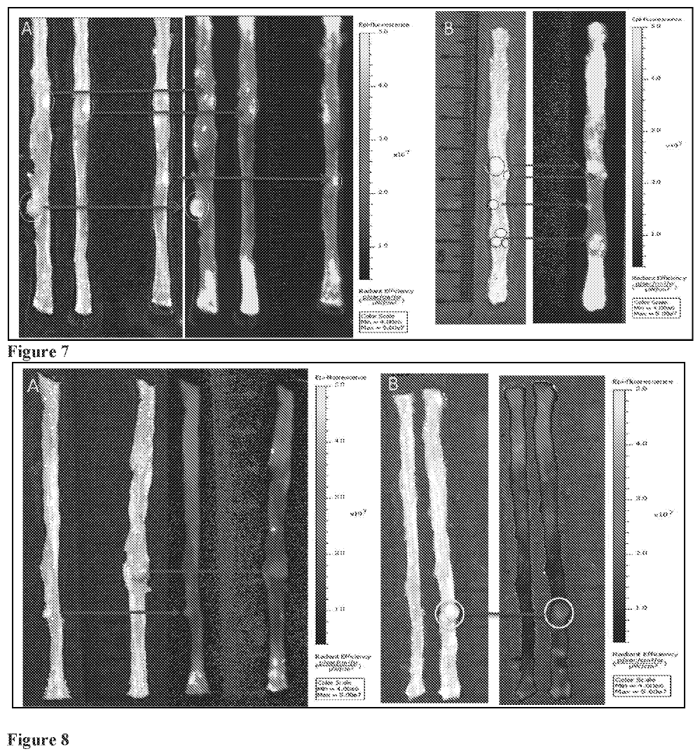

Ex-vivo and in vivo studies have been performed to evaluate the specificity of the IR680-SP-333 probe for colonic adenomas in Apc^{+/Min-FCCC} mice. IR680-SP-333 was prepared in DMEM/F-12 media containing 20 mM sodium phosphate buffer and 3% BSA, pH6; the same media used for in vitro experiments. Based on data from an initial pilot study, incubation of tissue with 0.1 μM IR680-SP-333 for 30 min at 37° C. was selected as the optimal condition for ex-vivo studies. Epifluorescence images were acquired as follows: excitation wavelength=640 nm, detection wavelength=700 nm, exposure time=auto-3 min, F-stop=1, binning=1 (“small” setting), field of view=0.3 cm (setting “C”). Although the IR680-SP-333 probe exhibited greater binding to colorectal polyps than to the surrounding normal colonic mucosa, the IR680-SP-333 probe did not bind to every colorectal polyp. To eliminate the contribution of the age of mouse to the observed heterogeneity in probe binding, an in depth imaging study was performed in mice of various ages (2, 3, 4, 5 and 6 months). In this ex-vivo study, mice were euthanized and colons excised, opened longitudinally, and washed with PBS. Colon tissues were incubated with 0.1 μM IR680-SP-333 at 37° C. for 30 min. After incubation, the tissues were washed with PBS (4 times) and imaged in the IVIS Spectrum, using the settings described above. Careful review of the resulting images lead to three major conclusions: 1) the binding of the IR680-SP-333 probe to colorectal polyps is size dependent. The probe signal was higher in polyps ≤3 mm in diameter than in those with a diameter >4 mm (FIG. 7). 2) because the signal varied with polyp size, binding was enhanced in mice 2-3 months of age where the majority of colorectal polyps were smaller (≤3 mm in diameter) (FIG. 7). As the mice aged, the colorectal polyps increased in size and the ability of probe to detect the polyps decreased (FIG. 8). These data were supported by Q-PCR analyses that revealed that GC-C expression was not always detectable in colonic adenomas. Because the incidence of colorectal polyps is higher in mice 3 months (70%) vs. 2 months (45%) of age, a decision was made to use mice 10-12 weeks of age for all subsequent in vivo IR680-SP-333 analyses to minimize the number of animal that needed to be screened for tumors. 3) signal of the IR680-SP-333 probe was high at the proximal and distal ends of the colon even in the absence of polyps. Similar nonspecific binding of unknown origin has been observed in previous experiments with independent probes at these same extreme regions.

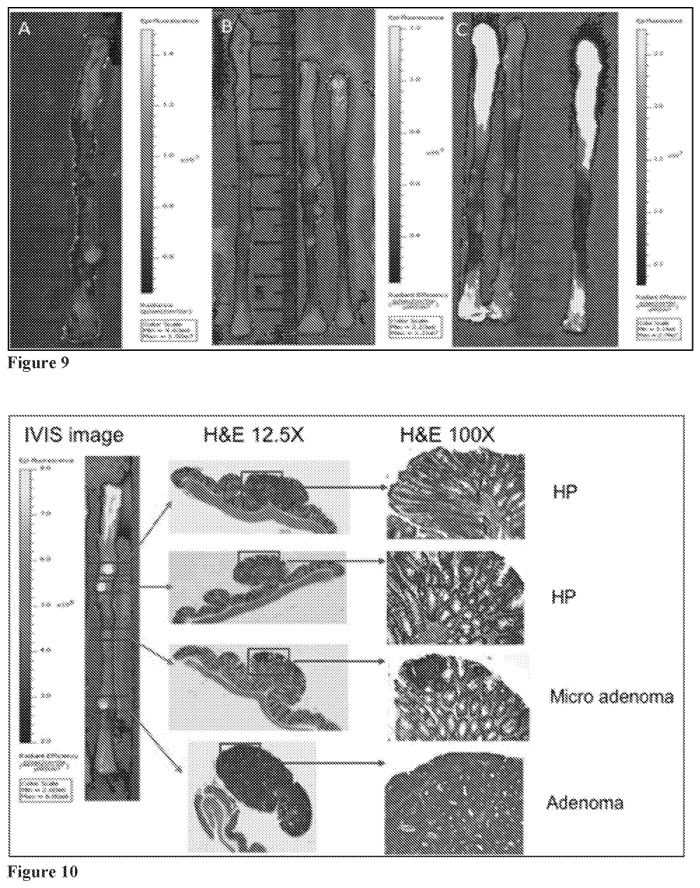

Following the ex-vivo binding studies, an experiment was performed to assess the ability of the probe to remain intact as it passed through the GI tract and bind to colonic lesions. In this in vivo experiment, Apc^{+/Min-FCCC} mice (10-12 wks of age) were subjected to a colonoscopic examination prior to probe administration, according to a protocol established previously (Hensley, Gastrointestinal Endoscopy group). Mice with confirmed colon polyps were randomized to receive IR680-SP-333 at 0.5 or 0.05 mg/kg body weight by gavage. Prior to probe administration, mice were fasted overnight and provided Pedialyte ad libitum for bowel cleansing. Mice were gavaged with IR680-SP-333 and euthanized at 30, 60, 90 and 120 min post treatment. At the time of euthanasia, the colon was excised, opened its entire length and rinsed with saline. Each colon was then placed on a plastic ruler and imaged in an IVIS Spectrum system using the settings described above for the ex-vivo experiments. Immediately following image acquisition, the entire colon was placed intact on a plastic ruler in 10% normal buffered formalin and areas positive and negative for probe signal were collected for histopathological evaluation. Comparison of images obtained from different time intervals revealed the best differential between the signal intensity of the colorectal polyps and the background signal of the adjacent normal colonic mucosa in mice gavaged with IR680-SP-333 0.05 mg/kg body weight and imaged at 30 min post treatment (FIG. 9A). Of note, a higher dose of SP-333-IR680 probe failed to improve the differential between tumor and normal tissue (FIG. 9B). The use of longer time interval (≥90 min) between dosing and imaging lead to an increase in nonspecific binding (FIG. 9C). Evaluation of additional doses of IR680-SP-333 probe (0.0158 and 0.158 mg/kg body weight) confirmed that gavaging mice with IR680-SP-333 at 0.05 mg/kg body weight and imaging 30 min post treatment yielded the greatest differential in signal between the non-neoplastic and neoplastic colonic mucosa.

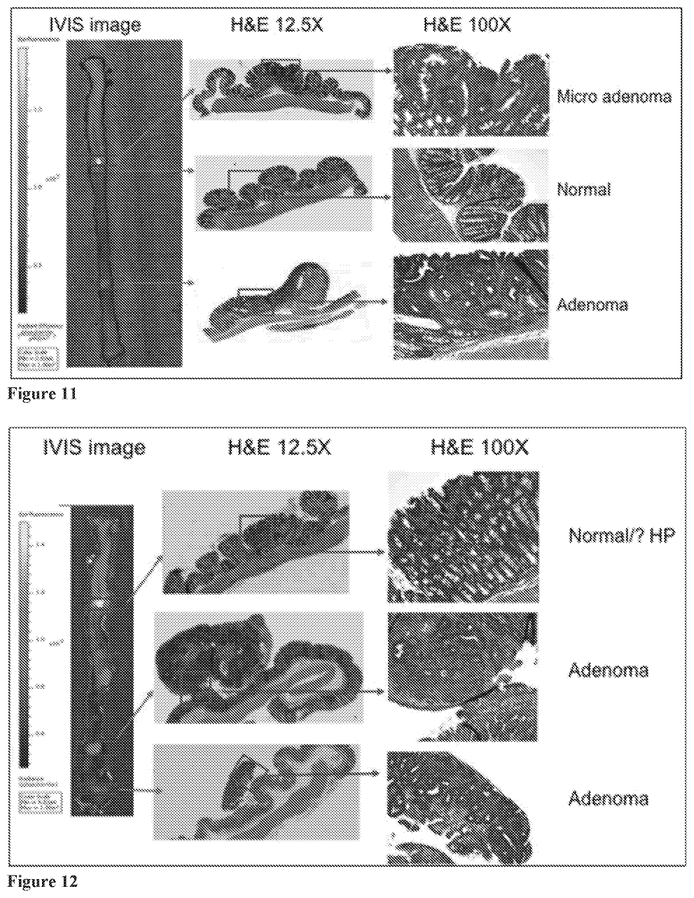

Colon tissues with strong signal (tumor) and corresponding background signal (nonneoplastic control) were processed for histopathology evaluation and reviewed bin a blinded manner. Two major conclusions were obtained: 1) the background signal of IR680-SP-333 in normal colonic mucosa varied between mice receiving the same treatment regimen (e.g. same dose of IR680-SP-333 and same post treatment time interval prior to imaging). This heterogeneity made it impossible to standardize the epi-fluorescence scale and classify positive and negative signals based solely on the epi-fluorescence value. Second, the intensity of the IR680-SP-333 probe signal did not correlate with either tumor size or the histological subtype of lesion (polyp, indeterminate or flat). As shown in FIG. 10 (mouse ID 12281), the strongest probe signal was observed in areas of hyperproliferation (HP) followed by an adenoma with intermediate signal intensity. A slight increase in signal was observed in a micro-adenoma as compared to the surrounding normal colonic mucosa. The colon displayed in FIG. 11 (mouse ID 12276) exhibits a very strong probe signal in small gross lesion (1-mm in size) and a weaker signal in a flat/indeterminate adenoma. In FIG. 12 (mouse ID 12201), strong signal is localized to a hyperplastic polyp (HP); an area of high proliferation; providing additional support for the lack of correlation between the intensity of the probe signal and size/type of colonic lesion. However, the accumulation of probe signal in areas of high proliferation (e.g. small lesions and hyperplastic polyps) suggest that IR-680-SP-333 may be useful in detecting early colorectal lesions. The probe IR-680-SP-333 may also be useful in detection of colorectal metastasis in liver and other organs.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A conjugate comprising: (a) a GCRA peptide; and (b) one or more components, to which the GCRA peptide is conjugated, wherein the one or more components are selected from the group consisting of a drug delivery vehicle, a chemotherapeutic agent, a micelle, a nanoparticle, a liposome, a polymer, a lipid, an imaging agent, and a labeling agent.

2. The conjugate of claim 1, wherein the peptide and the one or more components are covalently coupled directly or via a linker.

3. The conjugate of claim 2, wherein the linker is 4 to 10 carbons in length.

4. The conjugate of claim 2, wherein the GCRA peptide maintains biological activity.

5. The conjugate of claim 1, wherein the drug delivery vehicle is selected from the group consisting of liposomes, polymeric micelles, lipoprotein-based drug carriers, nanoparticle drug carriers, and dendrimers.

6. The conjugate of claim 1, wherein the component is a drug delivery vehicle and the conjugate further comprises at least one chemotherapeutic agent encapsulated within the drug delivery vehicle.

7. The conjugate of claim 6, wherein the at least one chemotherapeutic agent is a taxane, an anthracyclin, a platin or 5-fluorouracil and any combination thereof.

8. The conjugate of claim 6, wherein the drug delivery vehicle is a nanoparticle.

9. The conjugate of claim 8, wherein the nanoparticle is a PLA-based or a PLGA-based nanoparticle.

10. The conjugate of claim 1, wherein the component is conjugated to the N-terminus of the GCRA peptide.

11. The conjugate of claim 1, wherein the component is an imaging agent.

12. The conjugate of claim 1, wherein the imaging agent is a florescence dye or radioactive moiety.

13. The conjugate of claim 1, wherein the component is a labeling agent.

14. The conjugate of claim 13, wherein the labeling agent is biotin.

15. The conjugate of claim 14, wherein the biotin conjugate is followed by detection with avidin or streptavidin conjugated to fluorescence dye.

16. The conjugate of claim 1, wherein GCRA peptide is a peptide consisting essentially of the amino acid sequence of any one of Tables 1-8.

17. The conjugate of claim 16, wherein GCRA peptide is SEQ ID NO: 1 (SP-304), SEQ ID NO: 8 (SP-326), SEQ ID NO.:9 (SP-333), SP364 (SEQ ID NO.:100), or SP366 (SEQ ID NO.:102).

18. The conjugate of claim 16, wherein the GCRA peptide does not contain a D-amino acid at the N-terminus.

19. The conjugate of claim 16, wherein the GCRA peptide contains a D-amino acid at the C-terminus.

20. The conjugate of claim 16, wherein the GCRA peptide does not contain a D-amino acid at the N-terminus and contains a D-amino acid at the C-terminus.

21. The conjugate of claim 1, wherein the GCRA peptide is conjugated to two components.

22. The conjugate of claim 21, wherein the GCRA peptide is conjugated to a nanoparticle and an imaging agent.

23. The conjugate of claim 21, wherein the GCRA peptide is conjugated to a nanoparticle and a chemotherapeutic.

24. The conjugate of claims 21-23, wherein the nanoparticle is conjugated to the C-terminus of the GCRA peptide.

25. The conjugate of claim 22, wherein the imaging agent is conjugated to the N-terminus of the GCRA peptide and the nanoparticle is conjugated to the C-terminus of the GCRA peptide.

26. The conjugate of claim 23, wherein the chemotherapeutic agent is conjugated to the N-terminus of the GCRA peptide and the nanoparticle is conjugated to the C-terminus of the GCRA peptide.

27. A composition comprising the conjugate of any one of the preceding claims and a pharmaceutically acceptable excipient.

28. A method for the reduction of the incidence of colonic adenomas and colonic microadenomas in subject comprising administering to the subject a composition of claim 27, wherein the component is chemotherapeutic agent or a drug delivery vehicle, a micelle, a nanoparticle, a liposome, a polymer, a lipid having at least one chemotherapeutic agent encapsulated within.

29. A method for reducing the incidence of colon cancer in subject comprising administering to the subject a composition of claim 27, wherein the component is chemotherapeutic agent or a drug delivery vehicle, a micelle, a nanoparticle, a liposome, a polymer, a lipid having at least one chemotherapeutic agent encapsulated within.

30. A method for reducing the incidence of colon cancer metastasis in subject comprising administering to the subject a composition of claim 27, wherein the component is chemotherapeutic agent or a drug delivery vehicle, a micelle, a nanoparticle, a liposome, a polymer, a lipid having at least one chemotherapeutic agent encapsulated within.

31. A method of lowering the risk of adenoma development in subject comprising administering to the subject a composition of claim 27, wherein the component is chemotherapeutic agent or a drug delivery vehicle, a micelle, a nanoparticle, a liposome, a polymer, a lipid having at least one chemotherapeutic agent encapsulated within.

32. A method of treating colon cancer metastases in subject comprising administering to the subject a composition of claim 27, wherein the component is chemotherapeutic agent or a drug delivery vehicle, a micelle, a nanoparticle, a liposome, a polymer, a lipid having at least one chemotherapeutic agent encapsulated within.

33. The method of any one of claims 29-32, wherein the subject is at risk of developing such adenomas or microadenomas.

34. The method of claim 33, wherein the subject has been diagnosed with colon cancer, colonic adenomas, colonic microadenomas or colonic polyps or has a close blood relative diagnosed with colon cancer, colonic adenomas, colonic microadenomas or colonic polyps.

35. A method of detecting a colonic microadenoma or a flat colorectal dysplasias comprising contacting colonic tissue with a composition of claim 27, wherein the component is an imaging agent or labeling agent.

36. A method of detecting colorectal cancer metastasis or a flat colorectal dysplasias by administering to the subject a composition of claim 27, wherein the component is an imaging agent and detecting said imaging agent in the subject.
